SEQUENCE LISTING (79 sequences)

SEQ ID NO: 1 - protein, 532 aa, Homo sapiens
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 2 - protein, 1038 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 3 - protein, 647 aa, Homo sapiens
MAEEEAPKKSRAAGGGASWELCAGALSARLAEEGSGDAGGRRRPPVDPRRLARQLLLLLWLLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELRFSRTWIGIWSVLCCASTLFTVLTYLVDMRRFSYPERPIIFLSGCYTAVAVAYIAGFLLEDRVVCNDKFAEDGARTVAQGTKKEGCTILFMMLYFFSMASSIWWVILSLTWFLAAGMKWGHEAIEANSQYFHLAAWAVPAIKTITILALGQVDGDVLSGVCFVGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQAGGGAPPHPPMSPDFTVFMIKYLMTLIVGITSGFWIWSGKTLNSWRKFYTRLTNSKQGETTV

SEQ ID NO: 4 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 5 - protein, 704 aa, Homo sapiens
MVSWGRFICLVVVTMATLSLARPSFSLVEDTTLEPEGAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 6 - protein, 821 aa, Homo sapiens; identical to SEQ ID NO: 4

SEQ ID NO: 7 - protein, 769 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 8 - protein, 709 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 9 - protein, 707 aa, Homo sapiens
MVSWGRFICLVVVTMATLSLARPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEEKKVSGAVDCHKPPCNPSHLPCVLAVDQ

SEQ ID NO: 10 - protein, 706 aa, Homo sapiens
MVSWGRFICLVVVTMATLSLARPSFSLVEDTTLEPEGAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 11 - protein, 705 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 12 - protein, 704 aa, Homo sapiens; identical to SEQ ID NO: 5

SEQ ID NO: 13 - protein, 680 aa, Homo sapiens
MVSWGRFICLVVVTMATLSLARPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 14 - protein, 732 aa, Homo sapiens
MVSWGRFICLVVVTMATLSLARPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 15 - protein, 593 aa, Homo sapiens
MCLNKTKQLFGVAKRLPFWRKERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 16 - protein, 819 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 17 - protein, 1119 aa, Homo sapiens
MSAPSLRARAAGLGLLLCAVLGRAGRSDSGGRGELGQPSGVAAERPCPTTCRCLGDLLDCSRKRLARLPEPLPSWVARLDLSHNRLSFIKASSMSHLQSLREVKLNNNELETIPNLGPVSANITLLSLAGNRIVEILPEHLKEFQSLETLDLSSNNISELQTAFPALQLKYLYLNSNRVTSMEPGYFDNLANTLLVLKLNRNRISAIPPKMFKLPQLQHLELNRNKIKNVDGLTFQGLGALKSLKMQRNGVTKLMDGAFWGLSNMEILQLDHNNLTEITKGWLYGLLMLQELHLSQNAINRISPDAWEFCQKLSELDLTFNHLSRLDDSSFLGLSLLNTLHIGNNRVSYIADCAFRGLSSLKTLDLKNNEISWTIEDMNGAFSGLDKLRRLILQGNRIRSITKKAFTGLDALEHLDLSDNAIMSLQGNAFSQMKKLQQLHLNTSSLLCDCQLKWLPQWVAENNFQSFVNASCAHPQLLKGRSIFAVSPDGFVCDDFPKPQITVQPETQSAIKGSNLSFICSAASSSDSPMTFAWKKDNELLHDAEMENYAHLRAQGGEVMEYTTILRLREVEFASEGKYQCVISNHFGSSYSVKAKLTVNMLPSFTKTPMDLTIRAGAMARLECAAVGHPAPQIAWQKDGGTDFPAARERRMHVMPEDDVFFIVDVKIEDIGVYSCTAQNSAGSISANATLTVLETPSFLRPLLDRTVTKGETAVLQCIAGGSPPPKLNWTKDDSPLVVTERHFFAAGNQLLIIVDSDVSDAGKYTCEMSNTLGTERGNVRLSVIPTPTCDSPQMTAPSLDDDGWATVGVVIIAVVCCVVGTSLVWVVIIYHTRRRNEDCSITNTDETNLPADIPSYLSSQGTLADRQDGYVSSESGSHHQFVTSSGAGFFLPQHDSSGTCHIDNSSEADVEAATDLFLCPFLGSTGPMYLKGNVYGSDPFETYHTGCSPDPRTVLMDHYEPSYIKKKECYPCSHPSEESCERSFSNISWPSHVRKLLNTSYSHNEGPGMKNLCLNKSSLDFSANPEPASVASSNSFMGTFGKALRRPHLDAYSSFGQPSDCQPRAFYLKAHSSPDLDSGSEEDGKERTDFQEENHICTFKQTLENYRTPNFQSYDLDT

SEQ ID NO: 18 - protein, 494 aa, Homo sapiens
MRPPGFRNFLLLASSLLFAGLSAVPQSFSPSLRSWPGAACRLSRAESERRCRAPGQPPGAALCHGRGRCDCGVCICHVTEPGMFFGPLCECHEWVCETYDGSTCAGHGKCDCGKCKCDQGWYGDACQYPTNCDLTKKKSNQMCKNSQDIICSNAGTCHCGRCKCDNSDGSGLVYGKFCECDDRECIDDETEEICGGHGKCYCGNCYCKAGWHGDKCEFQCDITPWESKRRCTSPDGKICSNRGTCVCGECTCHDVDPTGDWGDIHGDTCECDERDCRAVYDRYSDDFCSGHGQCNCGRCDCKAGWYGKKCEHPQSCTLSAEESIRKCQGSSDLPCSGRGKCECGKCTCYPPGDRRVYGKTCECDDRRCEDLDGVVCGGHGTCSCGRCVCERGWFGKLCQHPRKCNMTEEQSKNLCESADGILCSGKGSCHCGKCICSAEEWYISGEFCDCDDRDCDKHDGLICTGNGICSCGNCECWDGWNGNACEIWLGSEYP

SEQ ID NO: 19 - protein, 317 aa, Homo sapiens [sequence omitted]

SEQ ID NO: 20 - protein, 580 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 21 - protein, 475 aa, Homo sapiens [sequence omitted]

SEQ ID NO: 22 - protein, 284 aa, Homo sapiens
GLPDYSAANPIKVTHRCYILENDTVQCDLDLYKSLQAWKDHKLHIDHEIETLQNKIKNLREVRGHLKKKRPEECDCHKISYHTQHKGRLKHRGSSLHPFRKGLQEKDKVWLLREQKRKKKLRKLLKRLQNNDTCSMPGLTCFTHDNQHWQTAPFWTLGPFCACTSANNNTYWCMRTINETHNFLFCEFATGFLEYFDLNTDPYQLMNAVNTLDRDVLNQLHVQLMELRSCKGYKQCNPRTRNMDLGLKDGGSYEQYRQFQRRKWPEMKRPSSKSLGQLWEGWEG

SEQ ID NO: 23 - protein, 1287 aa, Homo sapiens
MHPDLGPLCTLLYVTLTILCSSVSSDLAPYFTSEPLSAVQKLGGPVVLHCSAQPVTTRISWLHNGKTLDGNLEHVKIHQGTLTILSLNSSLLGYYQCLANNSIGAIVSGPATVSVAVLGDFGSSTKHVITAEEKSAGFIGCRVPESNPKAEVRYKIRGKWLEHSTENYLILPSGNLQILNVSLEDKGSYKCAAYNPVTHQLKVEPIGRKLLVSRPSSDDVHILHPTHSQALAVLSRSPVTLECVVSGVPAPQVYWLKDGQDIAPGSNWRRLYSHLATDSVDPADSGNYSCMAGNKSGDVKYVTYMVNVLEHASISKGLQDQIVSLGATVHFTCDVHGNPAPNCTWFHNAQPIHPSARHLTAGNGLKISGVTVEDVGMYQCVADNGIGFMHSTGRLEIENDGGFKPVIITAPVSAKVADGDFVTLSCNASGLPVPVIRWYDSHGLITSHPSQVLRSKSRKSQLSRPEGLNLEPVYFVLSQAGASSLHIQAVTQEHAGKYICEAANEHGTTQAEASLMVVPFETNTKAETVTLPDAAQNDDRSKRDGSETGLLSSFPVKVHPSAVESAPEKNASGISVPDAPIILSPPQTHTPDTYNLVWRAGKDGGLPINAYFVKYRKLDDGVGMLGSWHTVRVPGSENELHLAELEPSSLYEVLMVARSAAGEGQPAMLTFRTSKEKTASSKNTQASSPPVGIPKYPVVSEAANNNFGVVLTDSSRHSGVPEAPDRPTISTASETSVYVTWIPRANGGSPITAFKVEYKRMRTSNWLVAAEDIPPSKLSVEVRSLEPGSTYKFRVIAINHYGESFRSSASRPYQVVGFPNRFSSRPITGPHIAYTEAVSDTQIMLKWTYIPSSNNNTPIQGFYIYYRPTDSDNDSDYKRDVVEGSKQWHMIGHLQPETSYDIKMQCFNEGGESEFSNVMICETKVKRVPGASEYPVKDLSTPPNSLGSGGNVGPATSPARSSDMLYLIVGCVLGVMVLILMVFIAMCLWKNRQQNTIQKYDPPGYLYQGSDMNGQMVDYTTLSGASQINGNVHGGFLTNGGLSSGYSHLHHKVPNAVNGIVNGSLNGGLYSGHSNSLTRTHVDFEHPHHLVNGGGMYTAVPQIDPLECVNCRNCRNNNRCFTKTNSTFSSSPPPVVPVVAPYPQDGLEMKPLSHVKVPVCLTSAVPDCGQLPEESVKDNVEPVPTQRTCCQDIVNDVSSDGSEDPAEFSRGQEGMINLRIPDHLQLAKSCVWEGDSCAHSETEINIVSWNALILPPVPEGCAEKTMWSPPGIPLDSPTEVLQQPRET

SEQ ID NO: 24 - protein, 334 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQ

SEQ ID NO: 25 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 26 - protein, 581 aa, Homo sapiens
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 27 - protein, 115 aa, Homo sapiens
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 28 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRRGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 29 - protein, 1012 aa, Homo sapiens [sequence omitted]

SEQ ID NO: 30 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 31 - protein, 822 aa, Homo sapiens [sequence omitted]

SEQ ID NO: 32 - protein, 733 aa, Homo sapiens
MWSWKCLLFWAVLVTATLCTARPSPTLPEQDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 33 - protein, 731 aa, Homo sapiens
MWSWKCLLFWAVLVTATLCTARPSPTLPEQDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 34 - protein, 731 aa, Homo sapiens
MWSWKCLLFWAVLVTATLCTARPSPTLPEQDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 35 - protein, 729 aa, Homo sapiens
MWSWKCLLFWAVLVTATLCTARPSPTLPEQDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 36 - protein, 820 aa, Homo sapiens; identical to SEQ ID NO: 30

SEQ ID NO: 37 - protein, 812 aa, Homo sapiens
MAAVTRDFGEMLLHSGRVLPAEAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 38 - protein, 853 aa, Homo sapiens
MEARVSLKRRIELTVEYPWRCGALSPTSNCRTGMWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 39 - protein, 830 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQVLHPDLTTHLLPLRCFWKVRKRRCLLRGQEPHPCSRGAGQRVKAQQPSTMDGFLQGNRWGWAGEGAPT

SEQ ID NO: 40 - protein, 729 aa, Homo sapiens; identical to SEQ ID NO: 35

SEQ ID NO: 41 - protein, 828 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQVLHPDLTTHLLPLRCFWKVRKRRCLLRGQEPHPCSRGAGQRVKAQQPSTMDGFLQGNRWGWAGEGAPT

SEQ ID NO: 42 - protein, 741 aa, Homo sapiens
MWSWKCLLFWAVLVTATLCTARPSPTLPEQDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQVLHPDLTTHLLPLRCFWKVRKRRCLLRGQEPHPCSRGAGQRVKAQQPSTMDGFLQGNRWGWAGEGAPT

SEQ ID NO: 43 - protein, 1218 aa, Homo sapiens [sequence omitted]

SEQ ID NO: 44 - protein, 509 aa, Homo sapiens [sequence omitted]

SEQ ID NO: 45 - protein, 405 aa, Homo sapiens
MCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 46 - protein, 303 aa, Homo sapiens
MNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 47 - protein, 479 aa, Homo sapiens
MASTIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTGGAAFIFPNTSVYPEATQRITTRPDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY

SEQ ID NO: 48 - protein, 462 aa, Homo sapiens
MIQTVPDPAAHIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY

SEQ ID NO: 49 - protein, 486 aa, Homo sapiens
MIQTVPDPAAHIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTGGAAFIFPNTSVYPEATQRITTRPDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY

SEQ ID NO: 50 - protein, 387 aa, Homo sapiens
MVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTGGAAFIFPNTSVYPEATQRITTRPDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY

SEQ ID NO: 51 - protein, 363 aa, Homo sapiens
MVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY

SEQ ID NO: 52 - protein, 317 aa, Homo sapiens
MIQTVPDPAAHIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTGGAAFIFPNTSVYPEATQRITTRPDLPYEPPRRSAWTGHGHPTPQSKAAQPSPSTVPKTEDQRPQLDPYQILGPTSSRLANPGWTQ

SEQ ID NO: 53 - protein, 325 aa, Homo sapiens
MIQTVPDPAAHIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTGGAAFIFPNTSVYPEATQRITTRPGTKTPLCDLFIERHPRCPAEIRALSHVIQRELIPELKPVPDSLILPLLIWRLNPLKPFHSKTTLKELRAD

SEQ ID NO: 54 - protein, 455 aa, Homo sapiens
MASTIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY

SEQ ID NO: 55 - protein, 291 aa, Homo sapiens
MSMLPTFGFTQEQVACVCEVLQQGGNIERLGRFLWSLPACEHLHKNESVLKAKAVVAFHRGNFRELYKILESHQFSPHNHAKLQQLWLKAHYIEAEKLRGRPLGAVGKYRVRRKFPLPRSIWDGEETSYCFKEKSRSVLREWYAHNPYPSPREKRELAEATGLTTTQVSNWFKNRRQRDRAAEAKERENNENSNSNSHNPLNGSGKSVLGSSEDEKTPSGTPDHSSSSPALLLSPPPPGLPSLHSLGHPPGPSAVPVPVPGGGGADPLQHHHGLQDSILNPMSANLVDLGS

SEQ ID NO: 56 - protein, 293 aa, Homo sapiens
MSMLPTFGFTQEQVACVCEVLQQGGNIERLGRFLWSLPACEHLHKNESVLKAKAVVAFHRGNFRELYKILESHQFSPHNHAKLQQLWLKAHYIEAEKLRGRPLGAVGKYRVRRKFPLPRSIWDGEETSYCFKEKSRSVLREWYAHNPYPSPREKRELAEATGLTTTQVSNWFKNRRQRDRAAEAKERYEENNENSNSNSHNPLNGSGKSVLGSSEDEKTPSGTPDHSSSSPALLLSPPPPGLPSLHSLGHPPGPSAVPVPVPGGGGADPLQHHHGLQDSILNPMSANLVDLGS

SEQ ID NO: 57 - protein, 177 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 58 - protein, 175 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSR

SEQ ID NO: 59 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 60 - protein, 235 aa, Homo sapiens [sequence omitted]

SEQ ID NO: 61 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 62 - protein, 297 aa, Homo sapiens
MTSLPLGVKVEDSAFGKPAGGGAGQAPSAAAATAAAMGADEEGAKPKVSPSLLPFSVEALMADHRKPGAKESALAPSEGVQAAGGSAQPLGVPPGSLGAPDAPSSPRPLGHFSVGGLLKLPEDALVKAESPEKPERTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYGASGPFQRAALPVAPVGLYTAHVGYSMYHLT

SEQ ID NO: 63 - protein, 346 aa, Homo sapiens
MARPGQRWLGKWLVAMVVWALCRLATPLAKNLEPVSWSSLNPKFLSGKGLVIYPKIGDKLDIICPRAEAGRPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 64 - protein, 447 aa, Homo sapiens
MLLDAGPQYPAIGVTTFGASRHHSAGDVAERDVGLGINPFADGMGAFKLNPSSHELASAGQTAFTSQAPGYAAAAALGHHHHPGHVGSYSSAAFNSTRDFLFRNRGFGDAAAAASAQHSLFAASAGGFGGPHGHTDAAGHLLFPGLHEQAAGHASPNVVNGQMRLGFSGDMYPRPEQYGQVTSPRSEHYAAPQLHGYGPMNVNMAAHHGAGAFFRYMRQPIKQELICKWIEPEQLANPKKSCNKTFSTMHELVTHVTVEHVGGPEQSNHICFWEECPREGKPFKAKYKLVNHIRVHTGEKPFPCPFPGCGKVFARSENLKIHKRTHTGEKPFKCEFEGCDRRFANSSDRKKHMHVHTSDKPYLCKMCDKSYTHPSSLRKHMKVHESSSQGSQPSPAASSGYESSTPPTIVSPSTDNPTTSSLSPSSSAVHHTAGHSALSSNFNEWYV

SEQ ID NO: 65 - protein, 319 aa, Homo sapiens
MDPQNQHGSGSSLVVIQQPSLDSRQRLDYEREIQPTAILSLDQIKAIRGSNEYTEGPSVVKRPAPRTAPRQEKHERTHEIIPINVNNNYEHRHTSHLGHAVLPSNARGPILSRSTSTGSAASSGSNSSASSEQGLLGRSPPTRPVPGHRSERAIRTQPKQLIVDDLKGSLKEDLTQHKFICEQCGKCKCGECTAPRTLPSCLACNRQCLCSAESMVEYGTCMCLVKGIFYHCSNDDEGDSYSDNPCSCSQSHCCSRYLCMGAMSLFLPCLLCYPPAKGCLKLCRRCYDWIHRPGCRCKNSNTVYCKLESCPSRGQGKPS

SEQ ID NO: 66 - protein, 149 aa, Homo sapiens
MSLSFCGNNISSYNINDGVLQNSCFVDALNLVPHVFLLFITFPILFIGWGSQSSKVQIHHNTWLHFPGHNLRWILTFALLFVHVCEIAEGIVSDSRRESRHLHLFMPAVMGFVATTTSIVYYHNIETSNFPKLLLGFSHHESKSCYQHS

SEQ ID NO: 67 - protein, 548 aa, Homo sapiens
MKTPADTGFAFPDWAYKPESSPGSRQIQLWHFILELLRKEEYQGVIAWQGDYGEFVIKDPDEVARLWGVRKCKPQMNYDKLSRALRYYYNKRILHKTKGKRFTYKFNFNKLVLVNYPFIDVGLAGGAVPQSAPPVPSGGSHFRFPPSTPSEVLSPTEDPRSPPACSSSSSSLFSAVVARRLGRGSVSDCSDGTSELEEPLGEDPRARPPGPPDLGAFRGPPLARLPHDPGVFRVYPRPRGGPEPLSPFPVSPLAGPGSLLPPQLSPALPMTPTHLAYTPSPTLSPMYPSGGGGPSGSGGGSHFSFSPEDMKRYLQAHTQSVYNYHLSPRAFLHYPGLVVPQPQRPDKCPLPPMAPETPPVPSSASSSSSSSSSPFKFKLQRPPLGRRQRAAGEKAVAAADKSGGSAGGLAEGAGALAPPPPPPQIKVEPISEGESEEVEVTDISDEDEEDGEVFKTPRAPPAPPKPEPGEAPGASQCMPLKLRFKRRWSEDCRLEGGGGPAGGFEDEGEDKKVRGEGPGEAGGPLTPRRVSSDLQHATAQLSLEHRDS

SEQ ID NO: 68 - protein, 396 aa, Homo sapiens [sequence omitted]

SEQ ID NO: 69 - protein, 337 aa, Homo sapiens [sequence omitted]

SEQ ID NO: 70 - protein, 439 aa, Homo sapiens
MAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQLLRKAKENEEFLIGFNITSKGRQLPKRRLPFPEPYILVYANDAAISEPESVVSSLQGHRNFPTGTVPKWDSHIRAALSIERRKKRSTGVLLPLQNNELPGAEYQYKKDEVWEERKPYKTLQAQAPEKSKNKKKQRKGPHRKSQTLQFDEQTLKKARRKQWIEPRNCARRYLKSLKPSNHATIQSIVRAVGVVPGIPEPCCVPEKMSSLSILFFDENKNVVLKVYPNMTVESCACR

SEQ ID NO: 71 - protein, 455 aa, Homo sapiens
MREGRGGGREGRSAEPGPEARSHSVVPSRATHCCSFPEPFQQVCSRLAVKNHGLLLYALFSVILLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 72 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 73 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 74 - protein, 420 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKY

SEQ ID NO: 75 - protein, 238 aa, Homo sapiens [sequence omitted]

SEQ ID NO: 76 - protein, 260 aa, Homo sapiens
MDRHSSYIFIWLQLELCAMAVLLTKGEIRCYCDAAHCVATGYMCKSELSACFSRLLDPQNSNSPLTHGCLDSLASTTDICQAKQARNHSGTTIPTLECCHEDMCNYRGLHDVLSPPRGEASGQGNRYQHDGSRNLITKVQELTSSKELWFRAAVIAVPIAGGLILVLLIMLALRMLRSENKRLQDQRQQMLSRLHYSFHGHHSKKGQVAKLDLECMVPVSGHENCCLTCDKMRQADLSNDKILSLVHWGMYSGHGKLEFV

SEQ ID NO: 77 - protein, 685 aa, Homo sapiens [sequence omitted]

SEQ ID NO: 78 - protein, 1163 aa, Homo sapiens [sequence omitted]

SEQ ID NO: 79 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

(19) United States
(12) Patent Application Publication
(10) Pub. No.: US 2025/0137072 A1
(43) Pub. Date: May 1, 2025

(54) MARKERS FOR SKELETAL STEM CELL AND USES THEREOF

(71) Applicants: ADA Forsyth Institute, Inc., Cambridge, MA (US); University of Rochester, Rochester, NY (US)

(72) Inventors: Wei Hsu, Cambridge, MA (US); Zhirui Jiang, Cambridge, MA (US)

(73) Assignees: ADA Forsyth Institute, Inc., Cambridge, MA (US); University of Rochester, Rochester, NY (US)

(21) Appl. No.: 18/835,928

(22) PCT Filed: Feb. 16, 2023
(86) PCT No.: PCT/US23/62716

§ 371 Date: Aug. 5, 2024

Related U.S. Application Data
(60) Provisional application No. 63/311,692, filed on Feb. 18, 2022.

(51) Int. Cl. C12Q 1/6888; A61K 35/28; C12N 5/077

(52) U.S. Cl. CPC C12Q 1/6888; A61K 35/28; C12N 5/0654; C12N 5/0655; C12Q 2533/00

(57) ABSTRACT

This application relates to stem cell biology and regenerative medicine. Disclosed herein are methods for isolation of skeletal stem cells, related methods, related compositions, related products, and related uses.

### CROSS REFERENCE TO RELATED APPLICATION

This application claims priority to U.S. Provisional Application No. 63/311,692 filed on Feb. 18, 2022. The content of the application is incorporated herein by reference in its entirety.

### GOVERNMENT INTERESTS

This invention was made with government support under DE015654 and DE026936 awarded by the National Institutes of Health. The government has certain rights in the invention.

### FIELD OF THE INVENTION

This invention relates to stem cell biology and regenerative medicine.

### BACKGROUND

Skeletal stem cells (SSCs) are a type of self-renewing, multipotent, and skeletal lineage-committed progenitor cells that are capable of giving rise to cartilage, bone, and bone marrow stroma including marrow adipocytes and stromal cells. With the trilineage potentials to differentiate into chondrocytes, osteoblasts, marrow stromal cells, or adipocytes, SSCs play important roles in the development, homeostasis, and regeneration of bone tissues. For example, SSCs from the suture mesenchyme, which are referred to as suture stem cells (SuSCs), exhibit long-term self-renewal, clonal expansion, and multipotency. These SuSCs reside in the suture midline and serve as the skeletal stem cell population responsible for calvarial development, homeostasis, injury repair, and regeneration. Yet, the in vivo identity of SuSCs and SSCs remains largely elusive. There is a need for a cell marker, such as a cell surface marker, related cell identification and isolation methods.

### SUMMARY

This application addresses the need mentioned above in a number of aspects.

In one aspect, the present application provides a method for identifying or isolating or enriching a skeletal stem cell. The method comprises obtaining a start cell population; and identifying from the start cell population a first marker expressed by, on, or in a cell, wherein the first marker is selected from the group consisting of those in Tables 1 and 2. In some embodiments, the first marker is selected from the group consisting of those listed in Table 1. In some embodiments, the first marker is selected from the group consisting of Bmpr1a, Bmpr2, Fdz1, Fgfr2, Lrig3, Itgbl1, Apoe, Gpc3, Lpl, Sulf2, Cdon, Tgfbr2, Tgfbr3, Lrrc15, Mif, Axl, Itgav, Fgfr1, Jag1, Acvr1, Acvr2a, Acvr2b, Bmpr2, Erg, Six2, Pthlh, Twist1, Alpl, Msx1, Efnb1, Zic1, Spry1, Abcc9, Erf, Bmp2, Bmp3, Bmp4, Bmp5, Bmp6, Bmp7, Bmp8a, Bambi, Bmper, and Col3a1. In some embodiments, the first marker is selected from one, two, three, or four of Group 1, Group 2, Group 3, and Group 4 disclosed herein.

In one embodiment, the method further comprises isolating the cell expressing the first marker. In one embodiment, the method further comprises collecting a plurality of cells expressing the first marker to obtain an enriched skeletal stem cell population.

In some embodiments, the cell or cells can be identified, isolated, or enriched using a first agent (such as a first protein, a first polypeptide, a first nucleic acid, or a first composition) that specifically binds to the first marker.

In some embodiments, the method may further comprise identifying from the start cell population a second marker expressed by, on, or in the cell expressing the first marker. The second marker is different from the first marker. The second marker can be a marker selected from the group described above. In that case, the cell can be identified, isolated or enriched using a second agent (e.g., a second protein, a second polypeptide, a second nucleic acid, or a second composition) that specifically binds to the second marker. In some examples, the method can further comprise collecting a plurality of cells expressing the first and second markers to obtain an enriched skeletal stem cell population. In other examples, the method may further comprise identifying from the start cell population one or more additional markers expressed on or in the cell co-expressing both the first and the second markers. Each of the one or more additional markers may be individually identified using the same method as for the first and the second markers. The method therefore can result in a cell co-expressing the first, the second, and the one or more additional markers. In one embodiment, the method may further comprise collecting a plurality of cells expressing the first, the second, and the one or more markers to obtain an enriched skeletal stem cell population.

In each of the above-described methods, the start cell population can be from a tissue of a subject, such as a vertebrate, a mammal (including human and non-human mammal). The start cell population can comprise one or more selected from the group consisting of bone marrow, cord blood cells, embryonic stem cells or progenies thereof, mesenchymal stem cells or progenies thereof, and induced pluripotent stem cells (iPSCs) or progenies thereof. In a preferred embodiment, the mesenchymal stem cells are suture mesenchymal stem cells.

The present application further provides a composition comprising (i) a carrier and (ii) one or more cells identified, isolated, or enriched according to the method described above, or cells derived therefrom. The composition can be a pharmaceutical composition where the carrier is a pharmaceutically acceptable carrier. The composition can be an in vitro cell culture composition and the carrier can comprise a culture medium or a maintaining medium. The present application also provides a bone regeneration product or formulation comprising (i) the composition described above and (ii) a scaffold.

Also provided is a method for generating or regenerating cartilage or bone in a subject. The method comprises administering to a subject in need thereof an effective amount of the composition described above, or the bone regeneration product or formulation described above, at a site where regeneration of bone or cartilage is desired.

Further provided is a method of generating skeletal, stromal, or cartilaginous tissue. The method comprises (i) providing one or more cells obtained according to the method described above and (ii) inducing differentiation of the one or more cells, or differentiation of cells derived therefrom. The one or more cells may express a second, different marker selected from the group described above.

The details of one or more embodiments of the present application are set forth in the description below. Other features, objectives, and advantages of the present application will be apparent from the description and the claims.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIGS. 1A, 1B, and 1C show a single-cell RNA-sequencing analysis of mouse calvarial sutures.

FIGS. 1A and 1B show that heatmap (FIG. 1A) and tSNE plots (FIG. 1B) of integrated P10 coronal and lambdoid sutures datasets indicate the top 5 marker genes for each cell cluster and the grouping of suture cells into ten clusters (0-9), respectively.

FIG. 1C shows feature plots of the entire suture cells grouped into three major lineages. The Runx2+ skeletal cell lineages (Clusters 0, 1, 2, 4, and 9), Mcam+ vascular cell lineages (Clusters 7 and 8), and Rac2+ hematopoietic cell lineages (Clusters 3, 5, and 6) are outlined by blue, brown, and red broken lines, respectively. Color scales represent centered natural log transformation across cells.

FIGS. 2A, 2B, 2C, and 2D show the characterization of Runx2+ skeletal cell lineages in the suture mesenchyme.

FIG. 2A shows that the tSNE plot of cells from P10 coronal and lambdoid sutures shows Runx2+ skeletal lineages, outlined by the broken blue line. The Runx2+ lineage can be subdivided into five clusters (MC, PMC, OB, PCH, CH).

FIGS. 2B, 2C, and 2D show expression distributions of representative osteoblast (B), chondrocyte (C), and proliferation (D) gene markers project onto the tSNE plot. The MC clusters show low expression of osteoblast, chondrocyte, and proliferation genes. The proliferation marker genes classify the PMC and PCH clusters containing proliferating mesenchymal and chondrocyte cells. Color scales in (B), (C), and (D) represent centered natural log transformation across cells.

FIGS. 3A-3E show the characterization of Rac2+ hematopoietic cell lineages. Color scales in (B), (C), and (D) represent centered natural log transformation across cells.

FIG. 3A shows that the tSNE plot of cells from P10 coronal and lambdoid sutures containing Rac2+ hematopoietic cell lineages outlined by the broken red line can be subdivided into three clusters (HC1, HC2, and HC3).

FIGS. 3B and 3C show expression distributions of representative monocyte/macrophage and dendritic cell genes (B) and osteoclast genes (C) projected onto the tSNE plot classify the HC1 cluster.

FIG. 3D shows that the HC2 cluster represents T and B lymphocytes based on the projected distribution of lymphocyte genes.

FIG. 3E shows that the HC3 cluster contains NK cells and neutrophils expressing their specific genes.

FIGS. 4A and 4B show the characterization of Mcam+ vascular cell lineages. Color scales represent centered natural log transformation across cells.

FIG. 4A shows that the tSNE plot of cells from P10 coronal and lambdoid sutures containing Mcam+ vascular cell lineages outlined by the broken brown line can be subdivided into two clusters (VC1 and VC2).

FIG. 4B shows that expression distributions of representative endothelial cell markers projected onto the tSNE plot classify the VC1 cluster while the VC2 cluster contains cells expressing pericyte and vascular smooth muscle cell (SMC) markers.

FIGS. 5A-5H show the tSNE analysis of skeletal stem and precursor cell markers. Color scales represent centered natural log transformation across cells.

FIG. 5A shows that the tSNE and violin plots of Axin2, a marker for skeletal stem cells of calvarial suture, show expression in the MC cluster.

FIGS. 5B-5H show that other skeletal stem and precursor cell genes, Gli1 (B), Lepr (C), Prrx1 (D), Grem1 (E), Erg (F), Pthlh (G), and Six2 (H) also mark the MC cluster.

FIGS. 6A-6C show isolation of Axin2-expressing cells from the calvarial suture.

FIG. 6A shows that cells isolated from control or Axin2GFP mice are subjected to FACS-based sorting based on the parameters used to separate cells with differential expression of GFP.

FIG. 6B shows the post-sorting analysis demonstrating the success of separating cells that express high levels of GFP (population P5).

FIG. 6C shows graphs indicating the Axin2+ cell population consisting of approximately 3-7 percent in the entire suture cells in three independent experiments.

FIGS. 7A, 7B, and 7C show a quality control for scRNA-seq analysis of Axin2-expressing suture mesenchymal cells.

FIG. 7A shows that the nFeature_RNA and nCount-RNA indicate the number of genes and the total number of molecules detected within a cell, respectively, as well as the comparison of two independent datasets.

FIG. 7B shows UMAP plots illustrating the differential distribution of Axin2-expressing cells between two independent experiments and their division into seven subpopulations.

FIG. 7C shows heatmaps visualizing the most variant genes and top 5 DEGs for PCs in cell clusters based on PCA scores.

FIGS. 8A, 8B, and 8C show cell type-specific identification in the Axin2-expressing cells.

FIG. 8A shows UMAP plots demonstrating the distribution of 11,090 Axin2-expressing cells isolated from mouse calvarial suture and their visualization in 7 subclusters.

FIG. 8 B shows dot plots demonstrating cell type-specific markers enriched in each cluster.

FIG. 8C shows villon and UMAP plots indicating the enrichment of BMPR1A-expressing cells in the SC cluster. NEUT, neutrophils; DC, dendritic cells; PHC, proliferating hematopoietic cells; NK, natural killer cells; MONO, monocytes; SC, skeletogenic cells; LYM, lymphocytes. Color scales in (B) and (C) represent centered natural log transformation across cells.

FIG. 9 shows enriched expression of genes associated with human craniosynostosis in the SC cluster. Examination of a total of 83 genes whose mutation has been linked to suture synostosis in humans shows 27 of them exhibited enriched expression in the SC cluster. NEAT, neutrophils; DC, dendritic cells; PHC, proliferating hematopoietic cells; NK, natural killer cells; MONO, monocytes; SC, skeletogenic cells; LYM, lymphocytes.

FIGS. 10A, 10B, and 10C show reclustering analysis of skeletogenic cells in Axin2+ population.

FIG. 10A shows UMAP plots demonstrating the distribution of 4,696 Axin2-expressing cells isolated from mouse calvarial suture and their visualization in 5 subclusters.

FIG. 10B shows dot plots demonstrating cell type-specific markers enriched in each cluster. Color scale represents centered natural log transformation across cells.

FIG. 10C shows plots indicating the intracluster signaling activity of the BMP pathway and relevant BMP signaling molecules expressed in the SC cluster. MONO, monocytes; DC, dendritic cells; MP, macrophages; SC, skeletogenic cells; ENDO, endothelial cells.

FIG. 11 shows enriched expression of BMP signaling molecules in the SC cluster. Violin plots show the distribution of genes associated with BMP signaling in the SC cluster. MONO, monocytes; DC, dendritic cells; MP, macrophages; SC, skeletogenic cells; ENDO, endothelial cells.

FIG. 12 shows enriched distribution of genes associated with human craniosynostosis to the SC cluster. Examination of a total of 83 genes whose mutation has been linked to suture synostosis in humans shows 27 of them exhibited enriched expression in the SC cluster. MONO, monocytes; DC, dendritic cells; MP, macrophages; SC, skeletogenic cells; ENDO, endothelial cells.

### DETAILED DESCRIPTION OF THE INVENTION

This application relates to stem cell biology and regenerative medicine. Certain aspects of this invention are based, at least in part, on the identification and use of one or more cell markers, such as surface markers, of SSCs and/or SuSCs. The ability of SSCs and/or SuSCs to engraft in injury sites to replace the damaged skeleton can be used for stem cell-based therapy.

### Cells and Markers

Skeletal stem cells (SSCs) from the suture mesenchyme, also referred to as suture stem cells (SuSCs), exhibit long-term self-renewal, clonal expansion, and multipotency. These SuSCs reside in the suture midline and serve as the skeletal stem cell population responsible for calvarial development, homeostasis, injury repair, and regeneration. The ability of SuSCs to engraft in injury site to replace the damaged skeleton support their potential use for stem cell-based therapy.

Shown in Table 1 below are examples of certain preferred SSC markers. Exemplary amino acid sequences of these makers are listed below. Additional cell surface markers are listed in Table 2 below. Those in bold as shown in Table 2 are also listed in Table 1.

TABLE 1
Preferred SSC Markers

| Group 1 | | Group 2 | | Group 3 | | Group 4 | |
|---|---|---|---|---|---|---|---|
| Cell surface markers | | Craniosynostosis genes | | BMP signaling | | Non-cell surface markers | |
| Markers | SEQ ID NO | Markers | SEQ ID NO | Markers | SEQ ID NO | Markers | SEQ ID NO |
| Bmpr1a | 1 | Fgfr1 | 30 to 42 | Acvr1 | 44 | Erg | 47 to 54 |
| Bmpr2 | 2 | Jag1 | 43 | Acvr2a | 45 | Six2 | 55 and 56 |
| Fdz1 | 3 | | | Acvr2b | 46 | Pthlh | 57 and 58 |
| Fgfr2 | 4 to 16 | | | Bmpr2 | 2 | Twist1 | 59 and 60 |
| Lrig3 | 17 | | | | | Alpl | 61 |
| Itgbl1 | 18 | | | | | Msx1 | 62 |
| Apoe | 19 | | | | | Efnb1 | 63 |
| Gpc3 | 20 | | | | | Zic1 | 64 |
| Lpl | 21 | | | | | Spry1 | 65 |
| Sulf2 | 22 | | | | | Abcc9 | 66 |
| Cdon | 23 | | | | | Erf | 67 |
| Tgfbr2 | 24 | | | | | Bmp2 | 68 and 69 |
| Tgfbr3 | 25 | | | | | Bmp3 | 70 |
| Lrrc15 | 26 | | | | | Bmp4 | 71 |
| Mif | 27 | | | | | Bmp5 | 72 |
| Axl | 28 | | | | | Bmp6 | 73 |
| Itgav | 29 | | | | | Bmp7 | 74 |
| | | | | | | Bmp8a | 75 |
| | | | | | | Bambi | 76 |
| | | | | | | Bmper | 77 |
| | | | | | | COL3A1 | 78 and 79 |

Group 1 Cell surface marker genes expressed in the SC cluster
1. Bmpr1a
Protein sequence of human BMPR1A NCBI Reference Sequence:
NP_004320.2 (SEQ ID NO: 1)
MPQLYIYIRLLGAYLFIISRVQGQNLDSMLHGTGMKSDSDQKKSENGVTLAPEDTLPFLKCYCSGHCPDDAINNT
CITNGHCFAIIEEDDQGETTLASGCMKYEGSDFQCKDSPKAQLRRTIECCRTNLCNQYLQPTLPPVVIGPFFDGS
IRWLVLLISMAVCIIAMIIFSSCFCYKHYCKSISSRRRYNRDLEQDEAFIPVGESLKDLIDQSQSSGSGSGLPLL
VQRTIAKQIQMVRQVGKGRYGEVWMGKWRGEKVAVKVFFTTEEASWFRETEIYQTVLMRHENILGFIAADIKGTG
SWTQLYLITDYHENGSLYDFLKCATLDTRALLKLAYSAACGLCHLHTEIYGTQGKPAIAHRDLKSKNILIKKNGS
CCIADLGLAVKENSDTNEVDVPLNTRVGTKRYMAPEVLDESLNKNHFQPYIMADIYSFGLIIWEMARRCITGGIV
EEYQLPYYNMVPSDPSYEDMREVVCVKRLRPIVSNRWNSDECLRAVLKLMSECWAHNPASRLTALRIKKTLAKMV
ESQDVKI
2. Bmpr2 (SEQ ID NO: 2)

| | |
|---|---|
| 1 | mtsslqrpwr vpwlpwtill vstaaasqnq erlcafkdpy qqdlgigesr ishengtilc |
| 61 | skgstcyglw ekskgdinlv kqgcwshigd pqechyeecv vtttppsiqn gtyrfcccst |
| 121 | dlcnvnften fpppdttpls pphsfnrdet iiialasvsv lavlivalcf gyrmltgdrk |
| 181 | qglhsmnmme aaasepsldl dnlkllelig rgrygavykg slderpvavk vfsfanrqnf |
| 241 | inekniyrvp lmehdniarf ivgdervtad grmeyllvme yypngslcky lslhtsdwvs |
| 301 | scrlahsvtr glaylhtelp rgdhykpais hrdinsrnvl vkndgtcvis dfglsmrltg |
| 361 | nrlvrpgeed naaisevgti rymapevleg avnlrdcesa lkqvdmyalg liyweifmrc |
| 421 | tdlfpgesvp eyqmafqtev gnhptfedmq vlvsrekqrp kfpeawkens lavrslketi |
| 481 | edcwdqdaea rltaqcaeer maelmmiwer nksvsptvnp mstamqnern lshnrrvpki |
| 541 | gpypdyssss yiedsihhtd sivknisseh smsstpltig eknrnsinye rqqaqarips |
| 601 | petsvtslst nttttnttgl tpstgmttis empypdetnl httnvaqsig ptpvclqlte |
| 661 | edletnkldp kevdknlkes sdenlmehsl kqfsgpdpls stsssllypl iklaveatgq |
| 721 | qdftqtangq aclipdvlpt qiyplpkqqn lpkrptslpl ntknstkepr lkfgskhksn |
| 781 | lkqvetgvak mntinaaeph vvtvtmngva grnhsvnsha attqyangtv lsgqttnivt |
| 841 | hraqemlqnq figedtrlni nsspdehepl lrreqqaghd egvldrlvdr rerpleggrt |
| 901 | nsnnnnsnpc seqdvlaqgv pstaadpgps kprraqrpns ldlsatnvld gssiqigest |
| 961 | qdgksgsgek ikkrvktpys lkrwrpstwv istesldcev nnngsnravh sksstavyla |
| 1021 | eggtattmvs kdigmncl |

3. Fdz1 (SEQ ID NO: 3)

| | |
|---|---|
| 1 | maeeeapkks raagggaswe lcagalsarl aeegsgdagg rrrppvdprr larqlllllw |
| 61 | lleaplllgv ragaagqgpg qgpgpgqqpp pppqqqqsgq qyngergisv pdhgycqpis |
| 121 | iplctdiayn qtimpnllgh tnqedaglev hqfyplvkvq csaelkfflc smyapvctvl |
| 181 | eqalppcrsl cerarqgcea lmnkfgfqwp dtlkcekfpv hgagelcvgq ntsdkgtptp |
| 241 | sllpefwtsn pqhgggghrg gfpggagase rgkfscpral kvpsylnyhf lgekdcgapc |
| 301 | eptkvyglmy fgpeelrfsr twigiwsvlc castlftvlt ylvdmrrfsy perpiiflsg |
| 361 | cytavavayi agflledrvv cndkfaedga rtvaqgtkke gctilfmmly ffsmassiww |
| 421 | vilsltwfla agmkwgheai eansqyfhla awavpaikti tilalgqvdg dvlsgvcfvg |
| 481 | lnnvdalrgf vlaplfvylf igtsfllagf vslfrirtim khdgtktekl eklmvrigvf |
| 541 | svlytvpati viacyfyeqa frdqwerswv aqscksyaip cphlqaggga pphppmspdf |
| 601 | tvfmikylmt livgitsgfw iwsgktlnsw rkfytrltns kqgettv |

4. Fgfr2
FGFR2 [Homo sapiens] ACCESSION CAA96492 (SEQ ID NO: 4)

| | |
|---|---|
| 1 | mvswgrficl vvvtmatlsl arpsfslved ttlepeeppt kyqisqpevy vaapgeslev |
| 61 | rcllkdaavi swtkdgvhlg pnnrtvlige ylqikgatpr dsglyactas rtvdsetwyf |
| 121 | mvnvtdaiss gddeddtdga edfvsensnn krapywtnte kmekrlhavp aantvkfrcp |
| 181 | aggnpmptmr wlkngkefkq ehriggykvr nqhwslimes vvpsdkgnyt cvveneygsi |
| 241 | nhtyhldvve rsphrpilqa glpanastvv ggdvefvckv ysdaqphiqw ikhvekngsk |
| 301 | ygpdglpylk vlkaagvntt dkeievlyir nvtfedagey tclagnsigi sfhsawltvl |
| 361 | papgrekeit aspdyleiai ycigvfliac mvvtvilcrm knttkkpdfs sqpavhkltk |
| 421 | riplrrqvtv saessssmns ntplvrittr lsstadtpml agvseyelpe dpkwefprdk |
| 481 | ltlgkplgeg cfgqvvmaea vgidkdkpke avtvavkmlk ddatekdlsd lvsememmkm |
| 541 | igkhkniinl lgactqdgpl yviveyaskg nlreylrarr ppgmeysydi nrvpeeqmtf |
| 601 | kdlvsctyql argmeylasq kcihrdlaar nvlvtennvm kiadfglard innidyykkt |
| 661 | tngrlpvkwm apealfdrvy thqsdvwsfg vlmweiftlg gspypgipve elfkllkegh |
| 721 | rmdkpanctn elymmmrdcw havpsqrptf kqlvedldri ltlttneeyl dlsqpleqys |
| 781 | psypdtrssc ssgddsvfsp dpmpyepclp qyphingsvk t |

FGFR2 protein [Homo sapiens]
ACCESSION AAH39243 (SEQ ID NO: 5)

| | |
|---|---|
| 1 | mvswgrficl vvvtmatlsl arpsfslved ttlepegapy wtntekmekr lhavpaantv |
| 61 | kfrcpaggnp mptmrwlkng kefkgehrig gykvrnqhws limesvvpsd kgnytcvven |
| 121 | eygsinhtyh ldvversphr pilqaglpan astvvggdve fvckvysdaq phiqwikhve |
| 181 | kngskygpdg lpylkvlkaa gvnttdkeie vlyirnvtfe dageytclag nsigisfhsa |
| 241 | wltvlpapgr ekeitaspdy leiaiycigv fliacmvvtv ilcrmknttk kpdfssqpav |
| 301 | hkltkriplr rqvsaessss mnsntplvri ttrlsstadt pmlagvseye lpedpkwefp |
| 361 | rdkltlgkpl gegcfgqvvm aeavgidkdk pkeavtvavk mlkddatekd lsdlvsemem |
| 421 | mkmigkhkni inllgactqd gplyviveya skgnlreylr arrppgmeys ydinrvpeeq |
| 481 | mtfkdlvsct yqlargmeyl asqkcihrdl aarnvlvten nvmkiadfgl ardinnidyy |
| 541 | kkttngrlpv kwmapealfd rvythqsdvw sfgvlmweif tlggspypgi pveelfkllk |
| 601 | eghrmdkpan ctnelymmmr dcwhavpsqr ptfkqlvedl driltlttne eyldlsqple |
| 661 | qyspsypdtr sscssgddsv fspdpmpyep clpqyphing svkt |

fibroblast growth factor receptor 2 isoform 1 precursor [Homo sapiens]
ACCESSION NP_000132 (SEQ ID NO: 6)

| | |
|---|---|
| 1 | mvswgrficl vvvtmatlsl arpsfslved ttlepeeppt kyqisqpevy vaapgeslev |
| 61 | rcllkdaavi swtkdgvhlg pnnrtvlige ylqikgatpr dsglyactas rtvdsetwyf |
| 121 | mvnvtdaiss gddeddtdga edfvsensnn krapywtnte kmekrlhavp aantvkfrcp |
| 181 | aggnpmptmr wlkngkefkq ehriggykvr nqhwslimes vvpsdkgnyt cvveneygsi |
| 241 | nhtyhldvve rsphrpilqa glpanastvv ggdvefvckv ysdaqphiqw ikhvekngsk |
| 301 | ygpdglpylk vlkaagvntt dkeievlyir nvtfedagey tclagnsigi sfhsawltvl |
| 361 | papgrekeit aspdyleiai ycigvfliac mvvtvilcrm knttkkpdfs sqpavhkltk |
| 421 | riplrrqvtv saessssmns ntplvrittr lsstadtpml agvseyelpe dpkwefprdk |
| 481 | ltlgkplgeg cfgqvvmaea vgidkdkpke avtvavkmlk ddatekdlsd lvsememmkm |
| 541 | igkhkniinl lgactqdgpl yviveyaskg nlreylrarr ppgmeysydi nrvpeeqmtf |
| 601 | kdlvsctyql argmeylasq kcihrdlaar nvlvtennvm kiadfglard innidyykkt |
| 661 | tngrlpvkwm apealfdrvy thqsdvwsfg vlmweiftlg gspypgipve elfkllkegh |
| 721 | rmdkpanctn elymmmrdcw havpsqrptf kqlvedldri ltlttneeyl dlsqpleqys |
| 781 | psypdtrssc ssgddsvfsp dpmpyepclp qyphingsvk t |

fibroblast growth factor receptor 2 isoform 3 precursor [Homo sapiens]
ACCESSION NP_001138385 (SEQ ID NO: 7)

| | |
|---|---|
| 1 | mvswgrficl vvvtmatlsl arpsfslved ttlepeeppt kyqisqpevy vaapgeslev |
| 61 | rcllkdaavi swtkdgvhlg pnnrtvlige ylqikgatpr dsglyactas rtvdsetwyf |
| 121 | mvnvtdaiss gddeddtdga edfvsensnn krapywtnte kmekrlhavp aantvkfrcp |
| 181 | aggnpmptmr wlkngkefkq ehriggykvr nqhwslimes vvpsdkgnyt cvveneygsi |
| 241 | nhtyhldvve rsphrpilqa glpanastvv ggdvefvckv ysdaqphiqw ikhvekngsk |
| 301 | ygpdglpylk vlkhsginss naevlalfnv teadageyic kvsnyigqan qsawltvlpk |
| 361 | qqapgrekei taspdyleia iycigvflia cmvvtvilcr mknttkkpdf ssqpavhklt |
| 421 | kriplrrqvt vsaessssmn sntplvritt rlsstadtpm lagvseyelp edpkwefprd |
| 481 | kltlgkplge gcfgqvvmae avgidkdkpk eavtvavkml kddatekdls dlvsememmk |
| 541 | migkhkniin llgactqdgp lyviveyask gnlreylrar rppgmeysyd inrvpeeqmt |
| 601 | fkdlvsctyq largmeylas qkcihrdlaa rnvlvtennv mkiadfglar dinnidyykk |
| 661 | ttngrlpvkw mapealfdrv ythqsdvwsf gvlmweiftl ggspypgipv eelfkllkeg |
| 721 | hrmdkpanct nelymmmrdc whavpsqrpt fkqlvedldr iltlttnei |

fibroblast growth factor receptor 2 isoform 4 precursor [Homo sapiens]
ACCESSION NP_001138386 (SEQ ID NO: 8)

| | |
|---|---|
| 1 | mvswgrficl vvvtmatlsl arpsfslved ttlepeeppt kyqisqpevy vaapgeslev |
| 61 | rcllkdaavi swtkdgvhlg pnnrtvlige ylqikgatpr dsglyactas rtvdsetwyf |
| 121 | mvnvtdaiss gddeddtdga edfvsensnn krapywtnte kmekrlhavp aantvkfrcp |
| 181 | aggnpmptmr wlkngkefkq ehriggykvr nqhwslimes vvpsdkgnyt cvveneygsi |
| 241 | nhtyhldvva pgrekeitas pdyleiaiyc igvfliacmv vtvilcrmkn ttkkpdfssq |
| 301 | pavhkltkri plrrqvtvsa essssmnsnt plvrittrls stadtpmlag vseyelpedp |
| 361 | kwefprdklt lgkplgegcf gqvvmaeavg idkdkpkeav tvavkmlkdd atekdlsdlv |
| 421 | sememmkmig khkniinllg actqdgplyv iveyaskgnl reylrarrpp gmeysydinr |
| 481 | vpeeqmtfkd lvsctyqlar gmeylasqkc ihrdlaarnv lvtennvmki adfglardin |
| 541 | nidyykkttn grlpvkwmap ealfdrvyth qsdvwsfgvl mweiftlggs pypgipveel |
| 601 | fkllkeghrm dkpanctnel ymmmrdcwha vpsqrptfkq lvedldrilt lttneeyldl |
| 661 | sqpleqysps ypdtrsscss gddsvfspdp mpyepclpqy phingsvkt |

fibroblast growth factor receptor 2 isoform 5 precursor [Homo sapiens]
ACCESSION NP_001138387 (SEQ ID NO: 9)

| | |
|---|---|
| 1 | mvswgrficl vvvtmatlsl arpsfslved ttlepedais sgddeddtdg aedfvsensn |
| 61 | nkrapywtnt ekmekrlhav paantvkfrc paggnpmptm rwlkngkefk qehriggykv |
| 121 | rnqhwslime svvpsdkgny tcvveneygs inhtyhldvv ersphrpilq aglpanastv |
| 181 | vggdvefvck vysdaqphiq wikhvekngs kygpdglpyl kvlkaagvnt tdkeievlyi |
| 241 | rnvtfedage ytclagnsig isfhsawltv lpapgrekei taspdyleia iycigvflia |
| 301 | cmvvtvilcr mknttkkpdf ssqpavhklt kriplrrqvt vsaessssmn sntplvritt |
| 361 | rlsstadtpm lagvseyelp edpkwefprd kltlgkplge gcfgqvvmae avgidkdkpk |
| 421 | eavtvavkml kddatekdls dlvsememmk migkhkniin llgactqdgp lyviveyask |
| 481 | gnlreylrar rppgmeysyd inrvpeeqmt fkdlvsctyq largmeylas qkcihrdlaa |
| 541 | rnvlvtennv mkiadfglar dinnidyykk ttngrlpvkw mapealfdrv ythqsdvwsf |
| 601 | gvlmweiftl ggspypgipv eelfkllkeg hrmdkpanct nelymmmrdc whavpsqrpt |
| 661 | fkqlvedldr iltlttneee kkvsgavdch kppcnpshlp cvlavdq |

fibroblast growth factor receptor 2 isoform 6 precursor [Homo sapiens]
ACCESSION NP_001138388 (SEQ ID NO: 10)

| | |
|---|---|
| 1 | mvswgrficl vvvtmatlsl arpsfslved ttlepegapy wtntekmekr lhavpaantv |
| 61 | kfrcpaggnp mptmrwlkng kefkgehrig gykvrnqhws limesvvpsd kgnytcvven |
| 121 | eygsinhtyh ldvversphr pilqaglpan astvvggdve fvckvysdaq phiqwikhve |
| 181 | kngskygpdg lpylkvlkaa genttdkeie vlyirnvtfe dageytclag nsigisfhsa |
| 241 | wltvlpapgr ekeitaspdy leiaiycigv fliacmvvtv ilcrmknttk kpdfssqpav |
| 301 | hkltkriplr rqvtvsaess ssmnsntplv rittrlssta dtpmlagvse yelpedpkwe |
| 361 | fprdkltlgk plgegcfgqv vmaeavgidk dkpkeavtva vkmlkddate kdlsdlvsem |
| 421 | emmkmigkhk niinllgact qdgplyvive yaskgnlrey lrarrppgme ysydinrvpe |
| 481 | eqmtfkdlvs ctyqlargme ylasqkcihr dlaarnvlvt ennvmkiadf glardinnid |
| 541 | yykkttngrl pvkwmapeal fdrvythqsd vwsfgvlmwe iftlggspyp gipveelfkl |
| 601 | lkeghrmdkp anctnelymm mrdcwhavps qrptfkqlve dldriltltt neeyldlsqp |
| 661 | leqyspsypd trsscssgdd svfspdpmpy epclpqyphi ngsvkt |

fibroblast growth factor receptor 2 isoform 7 precursor [Homo sapiens]
ACCESSION NP_001138389 (SEQ ID NO: 11)

| | |
|---|---|
| 1 | mvswgrficl vvvtmatlsl arpsfslved ttlepeeppt kyqisqpevy vaapgeslev |
| 61 | rcllkdaavi swtkdgvhlg pnnrtvlige ylqikgatpr dsglyactas rtvdsetwyf |
| 121 | mvnvtdaiss gddeddtdga edfvsensnn krapywtnte kmekrlhavp aantvkfrcp |
| 181 | aggnpmptmr wlkngkefkq ehriggykvr nqhwslimes vvpsdkgnyt cvveneygsi |
| 241 | nhtyhldvve rsphrpilqa glpanastvv ggdvefvckv ysdaqphiqw ikhvekngsk |
| 301 | ygpdglpylk vlkvsaesss smnsntplvr ittrlsstad tpmlagvsey elpedpkwef |
| 361 | prdkltlgkp 1gegcfgqvv maeavgidkd kpkeavtvav kmlkddatek dlsdlvseme |
| 421 | mmkmigkhkn iinllgactq dgplyvivey askgnlreyl rarrppgmey sydinrvpee |
| 481 | qmtfkdlvsc tyqlargmey lasqkcihrd laarnvlvte nnvmkiadfg lardinnidy |
| 541 | ykkttngrlp vkwmapealf drvythqsdv wsfgvlmwei ftlggspypg ipveelfkll |
| 601 | keghrmdkpa nctnelymmm rdcwhavpsq rptfkqlved ldriltlttn eeyldlsqpl |
| 661 | egyspsypdt rsscssgdds vfspdpmpye pclpqyphin gsvkt |

fibroblast growth factor receptor 2 isoform 8 precursor [Homo sapiens]
ACCESSION NP_001138390 (SEQ ID NO: 12)

| | |
|---|---|
| 1 | mvswgrficl vvvtmatlsl arpsfslved ttlepegapy wtntekmekr lhavpaantv |
| 61 | kfrcpaggnp mptmrwlkng kefkgehrig gykvrnqhws limesvvpsd kgnytcvven |
| 121 | eygsinhtyh ldvversphr pilqaglpan astvvggdve fvckvysdaq phiqwikhve |
| 181 | kngskygpdg lpylkvlkaa gvnttdkeie vlyirnvtfe dageytclag nsigisfhsa |
| 241 | wltvlpapgr ekeitaspdy leiaiycigv fliacmvvtv ilcrmknttk kpdfssqpav |
| 301 | hkltkriplr rqvsaessss mnsntplvri ttrlsstadt pmlagvseye lpedpkwefp |
| 361 | rdkltlgkpl gegcfgqvvm aeavgidkdk pkeavtvavk mlkddatekd lsdlvsemem |
| 421 | mkmigkhkni inllgactqd gplyviveya skgnlreylr arrppgmeys ydinrvpeeq |
| 481 | mtfkdlvsct yqlargmeyl asqkcihrdl aarnvlvten nvmkiadfgl ardinnidyy |
| 541 | kkttngrlpv kwmapealfd rvythqsdvw sfgvlmweif tlggspypgi pveelfkllk |
| 601 | eghrmdkpan ctnelymmmr dcwhavpsqr ptfkqlvedl driltlttne eyldlsqple |
| 661 | qyspsypdtr sscssgddsv fspdpmpyep clpqyphing svkt |

fibroblast growth factor receptor 2 isoform 9 precursor [Homo sapiens]
ACCESSION NP_001138391 (SEQ ID NO: 13)

| | |
|---|---|
| 1 | mvswgrficl vvvtmatlsl arpsfslved ttlepedais sgddeddtdg aedfvsensn |
| 61 | nkrapywtnt ekmekrlhav paantvkfrc paggnpmptm rwlkngkefk qehriggykv |
| 121 | rnqhwslime svvpsdkgny tcvveneygs inhtyhldvv ersphrpilq aglpanastv |
| 181 | vggdvefvck vysdaqphiq wikhvekngs kygpdglpyl kvlkhsgins snaevlalfn |
| 241 | vteadageyi ckvsnyigqa nqsawltvlp kqqapgreke itaspdylei aiycigvfli |
| 301 | acmvvtvilc rmknttkkpd fssqpavhkl tkriplrrqv tvsaessssm nsntplvrit |
| 361 | trlsstadtp mlagvseyel pedpkwefpr dkltlgkplg egcfgqvvma eavgidkdkp |
| 421 | keavtvavkm lkddatekdl sdlvsememm kmigkhknii nllgactqdg plyviveyas |
| 481 | kgnlreylra rrppgmeysy dinrvpeeqm tfkdlvscty qlargmeyla sqkcihrdla |
| 541 | arnvlvtenn vmkiadfgla rdinnidyyk kttngrlpvk wmapealfdr vythqsdvws |
| 601 | fgvlmweift lggspypgip veelfkllke ghrmdkpanc tnelymmmrd cwhavpsqrp |
| 661 | tfkqlvedld riltlttnei |

fibroblast growth factor receptor 2 isoform 11 precursor [Homo sapiens]
ACCESSION NP_075418 (SEQ ID NO: 14)

| | |
|---|---|
| 1 | mvswgrficl vvvtmatlsl arpsfslved ttlepedais sgddeddtdg aedfvsensn |
| 61 | nkrapywtnt ekmekrlhav paantvkfrc paggnpmptm rwlkngkefk qehriggykv |
| 121 | rnqhwslime svvpsdkgny tcvveneygs inhtyhldvv ersphrpilq aglpanastv |
| 181 | vggdvefvck vysdaqphiq wikhvekngs kygpdglpyl kvlkaagvnt tdkeievlyi |
| 241 | rnvtfedage ytclagnsig isfhsawltv lpapgrekei taspdyleia iycigvflia |
| 301 | cmvvtvilcr mknttkkpdf ssqpavhklt kriplrrqvt vsaessssmn sntplvritt |
| 361 | rlsstadtpm lagvseyelp edpkwefprd kltlgkplge gcfgqvvmae avgidkdkpk |
| 421 | eavtvavkml kddatekdls dlvsememmk migkhkniin llgactqdgp lyviveyask |
| 481 | gnlreylrar rppgmeysyd inrvpeeqmt fkdlvsctyq largmeylas qkcihrdlaa |
| 541 | rnvlvtennv mkiadfglar dinnidyykk ttngrlpvkw mapealfdrv ythqsdvwsf |
| 601 | gvlmweiftl ggspypgipv eelfkllkeg hrmdkpanct nelymmmrdc whavpsqrpt |
| 661 | fkqlvedldr iltlttneey ldlsqpleqy spsypdtrss cssgddsvfs pdpmpyepcl |
| 721 | pqyphingsv kt |

fibroblast growth factor receptor 2 isoform 15 [Homo sapiens]
ACCESSION NP_001307583 XP_011537812 (SEQ ID NO: 15)

| | |
|---|---|
| 1 | mclnktkqlf gvakrlpfwr kersphrpil qaglpanast vvggdvefvc kvysdaqphi |
| 61 | qwikhvekng skygpdglpy lkvlkaagvn ttdkeievly irnvtfedag eytclagnsi |
| 121 | gisfhsawlt vlpapgreke itaspdylei aiycigvfli acmvvtvilc rmknttkkpd |
| 181 | fssqpavhkl tkriplrrqv tvsaessssm nsntplvrit trlsstadtp mlagvseyel |
| 241 | pedpkwefpr dkltlgkplg egcfgqvvma eavgidkdkp keavtvavkm lkddatekdl |
| 301 | sdlvsememm kmigkhknii nllgactqdg plyviveyas kgnlreylra rrppgmeysy |
| 361 | dinrvpeeqm tfkdlvscty qlargmeyla sqkcihrdla arnvlvtenn vmkiadfgla |
| 421 | rdinnidyyk kttngrlpvk wmapealfdr vythqsdvws fgvlmweift lggspypgip |
| 481 | veelfkllke ghrmdkpanc tnelymmmrd cwhavpsqrp tfkqlvedld riltlttnee |
| 541 | yldlsqpleq yspsypdtrs scssgddsvf spdpmpyepc lpqyphings vkt |

fibroblast growth factor receptor 2 isoform 16 precursor [Homo sapiens]
ACCESSION NP_001307587 XP_006717772 (SEQ ID NO: 16)

| | |
|---|---|
| 1 | mvswgrficl vvvtmatlsl arpsfslved ttlepeeppt kyqisqpevy vaapgeslev |
| 61 | rcllkdaavi swtkdgvhlg pnnrtvlige ylqikgatpr dsglyactas rtvdsetwyf |
| 121 | mvnvtdaiss gddeddtdga edfvsensnn krapywtnte kmekrlhavp aantvkfrcp |
| 181 | aggnpmptmr wlkngkefkq ehriggykvr nqhwslimes vvpsdkgnyt cvveneygsi |
| 241 | nhtyhldvve rsphrpilqa glpanastvv ggdvefvckv ysdaqphiqw ikhvekngsk |
| 301 | ygpdglpylk vlkaagvntt dkeievlyir nvtfedagey tclagnsigi sfhsawltvl |
| 361 | papgrekeit aspdyleiai ycigvfliac mvvtvilcrm knttkkpdfs sqpavhkltk |
| 421 | riplrrqvsa essssmnsnt plvrittrls stadtpmlag vseyelpedp kwefprdklt |
| 481 | lgkplgegcf gqvvmaeavg idkdkpkeav tvavkmlkdd atekdlsdlv sememmkmig |
| 541 | khkniinllg actqdgplyv iveyaskgnl reylrarrpp gmeysydinr vpeeqmtfkd |
| 601 | lvsctyqlar gmeylasqkc ihrdlaarnv lvtennvmki adfglardin nidyykkttn |
| 661 | grlpvkwmap ealfdrvyth qsdvwsfgvl mweiftlggs pypgipveel fkllkeghrm |
| 721 | dkpanctnel ymmmrdcwha vpsqrptfkq lvedldrilt lttneeyldl sqpleqysps |
| 781 | ypdtrsscss gddsvfspdp mpyepclpqy phingsvkt |

5. Lrig3 (SEQ ID NO: 17)

| | |
|---|---|
| 1 | msapslrara aglglllcav lgragrsdsg grgelgqpsg vaaerpcptt crclgdlldc |
| 61 | srkrlarlpe plpswvarld lshnrlsfik assmshlqsl revkinnnel etipnlgpvs |
| 121 | anitllslag nriveilpeh lkefqsletl dlssnnisel qtafpalqlk ylylnsnrvt |
| 181 | smepgyfdnl antllvlkln rnrisaippk mfklpqlqhl elnrnkiknv dgltfqglga |
| 241 | lkslkmqrng vtklmdgafw glsnmeilql dhnnlteitk gwlygllmlq elhlsqnain |
| 301 | rispdawefc qklseldltf nhlsrlddss flglslintl hignnrvsyi adcafrglss |
| 361 | lktldlknne iswtiedmng afsgldklrr lilqgnrirs itkkaftgld alehldlsdn |
| 421 | aimslqgnaf sqmkklqqlh lntssllcdc qlkwlpqwva ennfqsfvna scahpqllkg |
| 481 | rsifavspdg fvcddfpkpq itvqpetqsa ikgsnlsfic saasssdspm tfawkkdnel |
| 541 | lhdaemenya hlraqggevm eyttilrlre vefasegkyq cvisnhfgss ysvkakltvn |
| 601 | mlpsftktpm dltiragama rlecaavghp apqiawqkdg gtdfpaarer rmhvmpeddv |
| 661 | ffivdvkied igvysctaqn sagsisanat ltvletpsfl rplldrtvtk getavlqcia |
| 721 | ggspppklnw tkddsplvvt erhffaagnq lliivdsdvs dagkytcems ntlgtergnv |
| 781 | rlsviptptc dspqmtapsl dddgwatvgv viiavvccvv gtslvwvvii yhtrrrnedc |
| 841 | sitntdetnl padipsylss qgtladrqdg yvssesgshh qfvtssgagf flpqhdssgt |
| 901 | chidnssead veaatdlflc pflgstgpmy lkgnvygsdp fetyhtgcsp dprtvlmdhy |
| 961 | epsyikkkec ypcshpsees cersfsnisw pshvrklint syshnegpgm knlclnkssl |
| 1021 | dfsanpepas vassnsfmgt fgkalrrphl dayssfgqps dcqprafylk ahsspdldsg |
| 1081 | seedgkertd fqeenhictf kqtlenyrtp nfqsydldt |

6. Itgbl1 (SEQ ID NO: 18)

| | |
|---|---|
| 1 | mrppgfrnfl llassllfag lsavpqsfsp slrswpgaac rlsraeserr crapgqppga |
| 61 | alchgrgrcd cgvcichvte pgmffgplce chewvcetyd gstcaghgkc dcgkckcdqg |
| 121 | wygdacqypt ncdltkkksn qmcknsqdii csnagtchcg rckcdnsdgs glvygkfcec |
| 181 | ddreciddet eeicgghgkc ycgncyckag whgdkcefqc ditpweskrr ctspdgkics |
| 241 | nrgtcvcgec tchdvdptgd wgdihgdtce cderdcravy drysddfcsg hgqcncgrcd |
| 301 | ckagwygkkc ehpqsctlsa eesirkcqgs sdlpcsgrgk cecgkctcyp pgdrrvygkt |
| 361 | cecddrrced ldgvvcgghg tcscgrcvce rgwfgklcqh prkcnmteeq sknlcesadg |
| 421 | ilcsgkgsch cgkcicsaee wyisgefcdc ddrdcdkhdg lictgngics cgncecwdgw |
| 481 | ngnaceiwlg seyp |

7. Apoe (SEQ ID NO: 19)

| | |
|---|---|
| 1 | mkvlwaallv tflagcqakv eqavetepep elrqqtewqs gqrwelalgr fwdylrwvqt |
| 61 | lseqvqeell ssqvtqelra lmdetmkelk aykseleeql tpvaeetrar lskelqaaqa |
| 121 | rlgadmedvc grlvqyrgev qamlgqstee lrvrlashlr klrkrllrda ddlqkrlavy |
| 181 | qagaregaer glsairerlg plveqgrvra atvgslagqp lqeraqawge rlrarmeemg |
| 241 | srtrdrldev keqvaevrak leeqaqgirl qaeafqarlk swfeplvedm qrqwaglvek |
| 301 | vqaavgtsaa pvpsdnh |

8. Gpc3 (SEQ ID NO: 20)

| | |
|---|---|
| 1 | magtvrtacl vvamllsldf pgqaqppppp pdatchqvrs ffqrlqpglk wvpetpvpgs |
| 61 | dlqvclpkgp tccsrkmeek yqltarlnme qllqsasmel kfliiqnaav fqeafeivvr |
| 121 | haknytnamf knnypsltpq afefvgefft dvslyilgsd invddmvnel fdslfpviyt |
| 181 | qlmnpglpds aldineclrg arrdlkvfgn fpklimtqvs kslqvtrifl qalnlgievi |
| 241 | nttdhlkfsk dcgrmltrmw ycsycqglmm vkpcggycnv vmqgcmagvv eidkywreyi |
| 301 | lsleelvngm yriydmenvl lglfstihds iqyvqknagk ltttigklca hsqqrqyrsa |
| 361 | yypedlfidk kvlkvahveh eetlssrrre liqklksfis fysalpgyic shspvaendt |
| 421 | lcwngqelve rysqkaarng mknqfnlhel kmkgpepvvs qiidklkhin qllrtmsmpk |
| 481 | grvldknlde egfesgdcgd dedeciggsg dgmikvknql rflaelaydl dvddapgnsq |
| 541 | qatpkdneis tfhnlgnvhs plklltsmai svvcffflvh |

9. Lpl (SEQ ID NO: 21)

| | |
|---|---|
| 1 | meskallvlt lavwlqslta srggvaaadq rrdfidiesk falrtpedta edtchlipgv |
| 61 | aesvatchfn hssktfmvih gwtvtgmyes wvsklvaaly krepdsnviv vdwlsraqeh |
| 121 | ypvsagytkl vgqdvarfin wmeeefnypl dnvhllgysl gahaagiags ltnkkvnrit |
| 181 | gldpagpnfe yaeapsrlsp ddadfvdvlh tftrgspgrs igiqkpvghv diypnggtfq |
| 241 | pgcnigeair viaerglgdv dqlvkcsher fihlfidsll neenpskayr csskeafekg |
| 301 | lclscrknrc nnlgyeinkv rakrsskmyl ktrsqmpykv fhyqvkihfs gtesethtnq |
| 361 | afeislygtv aesenipftl pevstnktys fliytevdig ellmlklkwk sdsyfswsdw |
| 421 | wsspgfaiqk irvkagetqk kvifcsrekv shlqkgkapa vfvkchdksl nkksg |

10. Sulf2 (SEQ ID NO: 22)

| | |
|---|---|
| 1 | glpdysaanp ikvthrcyil endtvqcdld lykslqawkd hklhidheie tlqnkiknlr |
| 61 | evrghlkkkr peecdchkis yhtqhkgrlk hrgsslhpfr kglqekdkvw llreqkrkkk |
| 121 | lrkllkrlqn ndtcsmpglt cfthdnqhwq tapfwtlgpf cactsannnt ywcmrtinet |
| 181 | hnflfcefat gfleyfdlnt dpyqlmnavn tldrdvlngl hvqlmelrsc kgykqcnprt |
| 241 | rnmdlglkdg gsyeqyrqfq rrkwpemkrp sskslgqlwe gweg |

11. Cdon (SEQ ID NO: 23)

| | |
|---|---|
| 1 | mhpdlgplct llyvtltilc ssvssdlapy ftseplsavq klggpvvlhc saqpvttris |
| 61 | wlhngktldg nlehvkihqg tltilslnss llgyyqclan nsigaivsgp atvsvavlgd |
| 121 | fgsstkhvit aeeksagfig crvpesnpka evrykirgkw lehstenyli lpsgnlqiln |
| 181 | vsledkgsyk caaynpvthq lkvepigrkl lvsrpssddv hilhpthsqa lavlsrspvt |
| 241 | lecvvsgvpa pqvywlkdgq diapgsnwrr lyshlatdsv dpadsgnysc magnksgdvk |
| 301 | yvtymvnvle hasiskglqd qivslgatvh ftcdvhgnpa pnctwfhnaq pihpsarhlt |
| 361 | agnglkisgv tvedvgmyqc vadngigfmh stgrleiend ggfkpviita pvsakvadgd |
| 421 | fvtlscnasg lpvpvirwyd shglitshps qvlrsksrks qlsrpeglnl epvyfvlsqa |
| 481 | gasslhiqav tqehagkyic eaanehgttq aeaslmvvpf etntkaetvt lpdaaqnddr |
| 541 | skrdgsetgl lssfpvkvhp savesapekn asgisvpdap iilsppqtht pdtynlvwra |
| 601 | gkdgglpina yfvkyrkldd gvgmlgswht vrvpgsenel hlaelepssl yevlmvarsa |
| 661 | agegqpamlt frtskektas skntqasspp vgipkypvvs eaannnfgvv ltdssrhsgv |
| 721 | peapdrptis tasetsvyvt wipranggsp itafkveykr mrtsnwlvaa edippsklsv |
| 781 | evrslepgst ykfrviainh ygesfrssas rpyqvvgfpn rfssrpitgp hiayteavsd |
| 841 | tqimlkwtyi pssnnntpiq gfyiyyrptd sdndsdykrd vvegskqwhm ighlqpetsy |
| 901 | dikmqcfneg gesefsnvmi cetkvkrvpg aseypvkdls tppnslgsgg nvgpatspar |
| 961 | ssdmlylivg cvlgvmvlil mvfiamclwk nrqqntiqky dppgylyqgs dmngqmvdyt |
| 1021 | tlsgasqing nvhggfltng glssgyshlh hkvpnavngi vngslnggly sghsnsltrt |
| 1081 | hvdfehphhl vngggmytav pqidplecvn crncrnnnrc ftktnstfss spppvvpvva |
| 1141 | pypqdglemk plshvkvpvc ltsavpdcgq lpeesvkdnv epvptqrtcc qdivndvssd |
| 1201 | gsedpaefsr gqegminlri pdhlqlaksc vwegdscahs eteinivswn alilppvpeg |
| 1261 | caektmwspp gipldsptev lqqpret |

12. Tgfbr2 (SEQ ID NO: 24)

| | |
|---|---|
| 1 | mgrgllrglw plhivlwtri astipphvqk svnndmivtd nngavkfpql ckfcdvrfst |
| 61 | cdnqkscmsn csitsicekp qevcvavwrk ndenitletv chdpklpyhd filedaaspk |
| 121 | cimkekkkpg etffmcscss decndniifs eeyntsnpdl llvifqvtgi slipplgvai |
| 181 | sviiifycyr vnrqqklsst wetgktrklm efsehcaiil eddrsdisst canninhnte |
| 241 | llpieldtlv gkgrfaevyk aklkqntseq fetvavkifp yeeyaswkte kdifsdinlk |
| 301 | henilqflta eerktelgkq ywlitafhak gnlq |

13. Tgfbr3 (SEQ ID NO: 25)

| | |
|---|---|
| 1 | mtshyviaif almssclata gpepgalcel spvsashpvq almesftvls gcasrgttgl |
| 61 | pqevhvlnlr tagqgpgqlq revtlhlnpi ssvhihhksv villnsphpl vwhlkterla |
| 121 | tgvsrlflvs egsvvqfssa nfsltaetee rnfphgnehl lnwarkeyga vtsftelkia |
| 181 | rniyikvged qvfppkcnig knflslnyla eylqpkaaeg cvmssqpqne evhiielitp |
| 241 | nsnpysafqv ditidirpsq edlevvknli lilkckksvn wviksfdvkg slkiiapnsi |
| 301 | gfgkesersm tmtksirddi pstqgnlvkw aldngyspit sytmapvanr fhlrlennee |
| 361 | mgdeevhtip pelrilldpg alpalqnppi rggegqnggl pfpfpdisrr vwneegedgl |
| 421 | prpkdpvips iqlfpglrep eevqgsvdia lsvkcdnekm ivavekdsfq asgysgmdvt |
| 481 | lldptckakm ngthfvlesp lngcgtrprw saldgvvyyn siviqvpalg dssgwpdgye |
| 541 | dlesgdngfp gdmdegdasl ftrpeivvfn cslqqvrnps sfqeqphgni tfnmelyntd |
| 601 | lflvpsqgvf svpenghvyv evsvtkaeqe lgfaiqtcfi spysnpdrms hytiienicp |
| 661 | kdesvkfysp krvhfpipqa dmdkkrfsfv fkpvfntsll flqceltlct kmekhpqklp |
| 721 | kcvppdeact sldasiiwam mqnkktftkp lavihheaes kekgpsmkep npisppifhg |
| 781 | ldtltvmgia faafvigall tgalwyiysh tgetagrqqv ptsppasens saahsigstq |
| 841 | stpcssssta |

14. Lrrc15 (SEQ ID NO: 26)

| | |
|---|---|
| 1 | mplkhyllll vgcqawgagl ayhgcpsect csrasqvect garivavptp lpwnamslqi |
| 61 | lnthitelne spflnisali alrieknels ritpgafrnl gslrylslan nklqvlpigl |
| 121 | fqgldslesl llssnqllqi qpahfsqcsn lkelqlhgnh leyipdgafd hlvgltklnl |
| 181 | gknslthisp rvfqhlgnlq vlrlyenrlt dipmgtfdgl vnlqelalqq nqigllspgl |
| 241 | fhnnhnlqrl ylsnnhisql ppsvfmqlpq lnrltlfgns lkelspgifg pmpnlrelwl |
| 301 | ydnhisslpd nvfsnlrqlq vlilsrnqis fispgafngl telrelslht nalqdldgnv |
| 361 | frmlanlqni slqnnrlrql pgnifanvng lmaiqlqnnq lenlplgifd hlgklcelrl |
| 421 | ydnpwrcdsd ilplrnwill nqprlgtdtv pvcfspanvr gqsliiinvn vavpsvhvpe |
| 481 | vpsypetpwy pdtpsypdtt svssttelts pvedytdltt iqvtddrsvw gmtqaqsgla |
| 541 | iaaivigiva lacslaacvg cccckkrsqa vlmqmkapne c |

15. Mif (SEQ ID NO: 27)

| | |
|---|---|
| 1 | mpmfivntnv prasvpdgfl seltqqlaga tgkppqyiav hvvpdqlmaf ggssepcalc |
| 61 | slhsigkigg aqnrsyskll cgllaerlri spdrvyinyy dmnaanvgwn nstfa |

16. Axl (SEQ ID NO: 28)

| | |
|---|---|
| 1 | mawrcprmgr vplawclalc gwacmaprgt qaeespfvgn pgnitgargl tgtlrcqlqv |
| 61 | qgeppevhwl rdgqilelad stqtqvplge deqddwivvs qlritslqls dtgqyqclvf |
| 121 | lghqtfvsqp gyvgleglpy fleepedrtv aantpfnlsc qaqgppepvd llwlqdavpl |
| 181 | atapghgpqr slhvpglnkt ssfsceahna kgvttsrtat itvlpqqprn 1hlvsrqpte |
| 241 | levawtpgls giyplthctl qavlsddgmg iqagepdppe epltsqasvp phqlrlgslh |
| 301 | phtpyhirva ctssqgpssw thwlpvetpe gvplgppeni satrngsqaf Vhwqeprapl |
| 361 | qgtllgyrla yqgqdtpevl mdiglrqevt lelqgdgsvs nltvcvaayt aagdgpwslp |
| 421 | vpleawrpgq aqpvhqlvke pstpafswpw wyvllgavva aacvlilalf lvhrrkketr |
| 481 | ygevfeptve rgelvvryrv rksysrrtte atinslgise elkeklrdvm vdrhkvalgk |
| 541 | tlgegefgav megqlnqdds ilkvavktmk iaictrsele dflseavcmk efdhpnvmrl |
| 601 | igvcfqgser esfpapvvil pfmkhgdlhs fllysrlgdq pvylptqmlv kfmadiasgm |
| 661 | eylstkrfih rdlaarncml nenmsvcvad fglskkiyng dyyrqgriak mpvkwiaies |
| 721 | ladrvytsks dvwsfgvtmw eiatrgqtpy pgvenseiyd ylrrgnrlkq padcldglya |
| 781 | lmsrcwelnp qdrpsftelr edlentlkal ppaqepdeil yvnmdegggy peppgaagga |
| 841 | dpptqpdpkd scscltaaev hpagryvlcp sttpspaqpa drgspaapgq edga |

17. Itgav (SEQ ID NO: 29)

| | |
|---|---|
| 1 | mafpprrrlr lgprglplll sglllplcra fnldvdspae ysgpegsyfg favdffvpsa |
| 61 | ssrmfllvga pkanttqpgi veggqvlkcd wsstrrcqpi efdatgnrdy akddplefks |
| 121 | hqwfgasvrs kqdkilacap lyhwrtemkq erepvgtcfl qdgtktveya persrqlisd |
| 181 | qvaeivskyd pnvysikynn qlatrtaqai fddsylgysv avgdfngdgi ddfvsgvpra |
| 241 | artlgmvyiy dgknmsslyn ftgeqmaayf gfsvaatdin gddyadvfig aplfmdrgsd |
| 301 | gklqevgqvs vslqrasgdf qttklngfev farfgsaiap lgdldqdgfn diaiaapygg |
| 361 | edkkgivyif ngrstglnav psqilegqwa arsmppsfgy smkgatdidk ngypdlivga |
| 421 | fgvdrailyr arpvitvnag levypsilnq dnktcslpgt alkvscfnvr fclkadgkgv |
| 481 | lprklnfqve llldklkqkg airralflys rspshsknmt isrgglmqce eliaylrdes |
| 541 | efrdkltpit ifmeyrldyr taadttglqp ilnqftpani srqahilldc gednvckpkl |
| 601 | evsvdsdqkk iyigddnplt livkaqnqge gayeaelivs iplqadfigv vrnnealarl |
| 661 | scalktenqt rqvvcdlgnp mkagtqllag lrfsvhqqse mdtsvkfdlq iqssnlfdkv |
| 721 | spvvshkvdl avlaaveirg vsspdhiflp ipnwehkenp eteedvgpvv qhiyelrnng |
| 781 | pssfskamlh lqwpykynnn tllyilhydi dgpmnctsdm einplrikis slqttekndt |
| 841 | vagqgerdhl itkrdlalse gdihtlgcgv aqclkivcqv grldrgksai lyvksllwte |
| 901 | timnkenqnh syslkssasf nviefpyknl pieditnstl vttnvtwgiq papmpvpvwv |
| 961 | iilavlagll llavlvfvmy rmgffkrvrp pqeegereql qphengegns et |

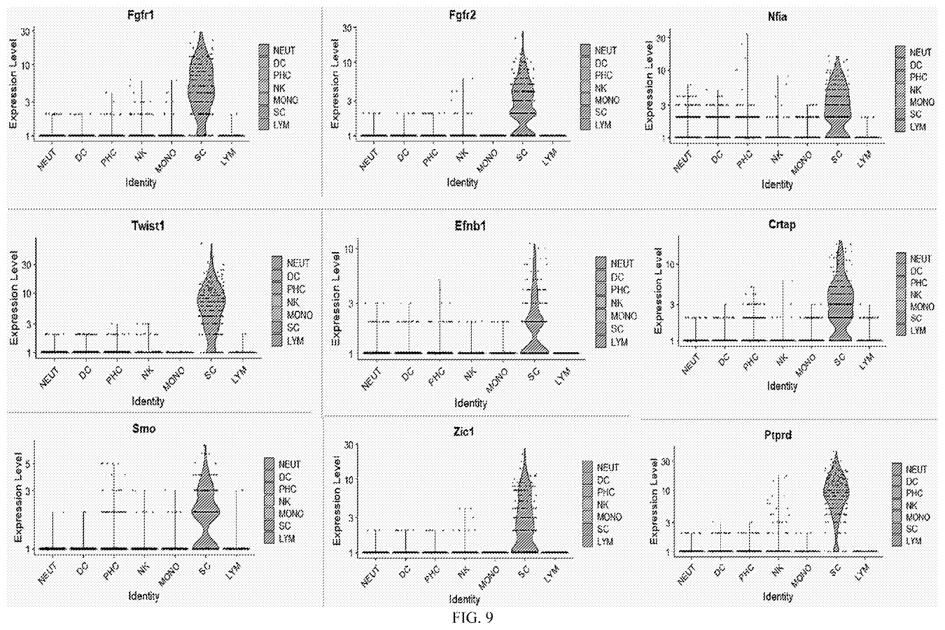

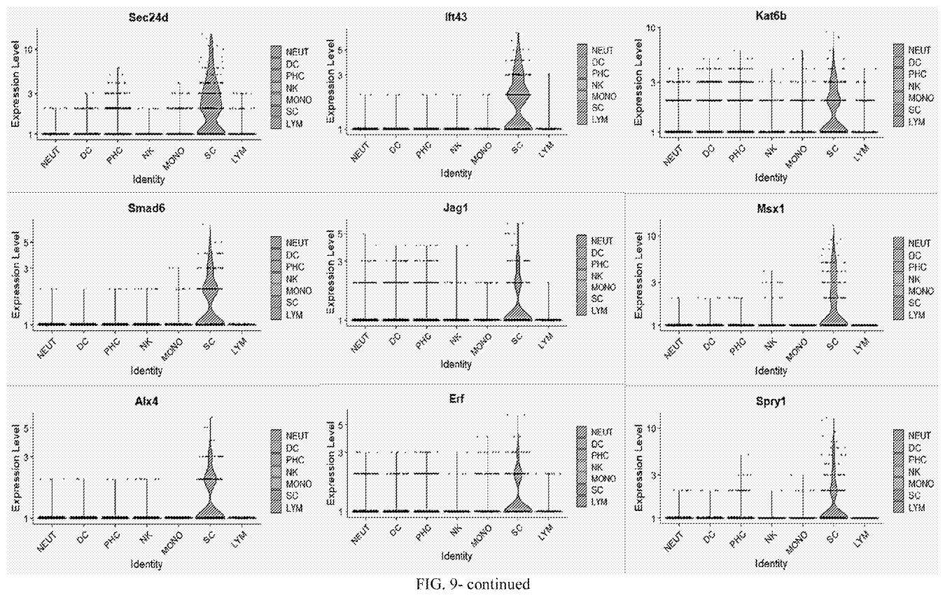

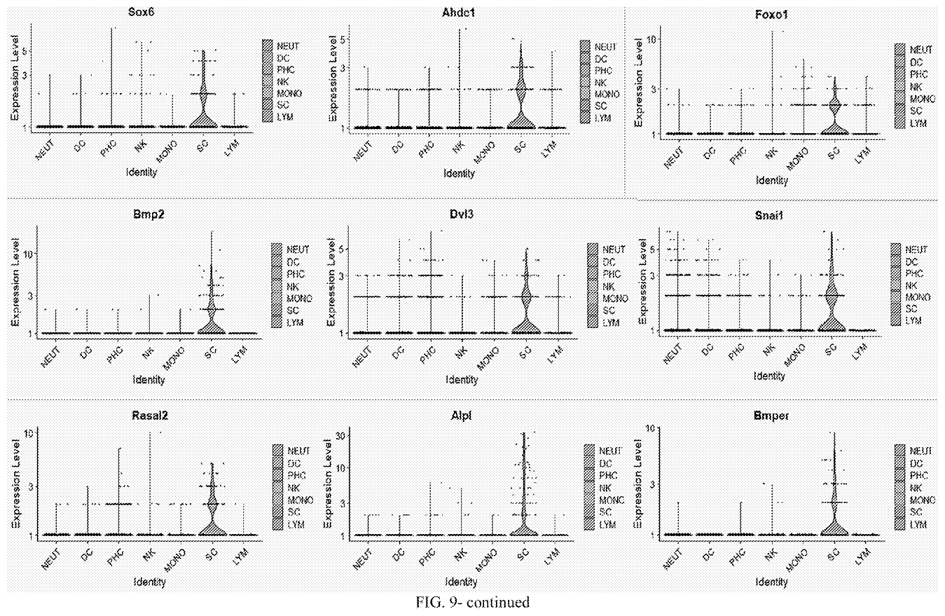

Group 2 From FIG. 9 & 12 (Craniosynostosis genes):
18. Fgfr1 (SEQ ID NO: 30)
FGFR1 protein [Homo sapiens]
ACCESSION AAH15035

| | |
|---|---|
| 1 | mwswkcllfw avlvtatlct arpsptlpeq aqpwgapvev esflvhpgdl lqlrcrlrdd |
| 61 | vqsinwlrdg vqlaesnrtr itgeevevqd svpadsglya cvtsspsgsd ttyfsvnvsd |
| 121 | alpssedddd dddssseeke tdntkpnrmp vapywtspek mekklhavpa aktvkfkcps |
| 181 | sgtpnptlrw lkngkefkpd hriggykvry atwsiimdsv vpsdkgnytc iveneygsin |
| 241 | htyqldvver sphrpilqag lpanktvalg snvefmckvy sdpqphiqwl khievngski |
| 301 | gpdnlpyvqi lktagvnttd kemevlhlrn vsfedageyt clagnsigls hhsawltvle |
| 361 | aleerpavmt splyleiiiy ctgafliscm vgsvivykmk sgtkksdfhs qmavhklaks |
| 421 | iplrrqvsad ssasmnsgvl lvrpsrlsss gtpmlagvse yelpedprwe lprdrlvlgk |
| 481 | plgegcfgqv vlaeaigldk dkpnrvtkva vkmlksdate kdlsdlisem emmkmigkhk |
| 541 | niinllgact qdgplyvive yaskgnlrey lqarrppgle ycynpshnpe eqlsskdlvs |
| 601 | cayqvargme ylaskkcihr dlaarnvlvt ednvmkiadf glardihhid yykkttngrl |
| 661 | pvkwmapeal fdriythqsd vwsfgvllwe iftlggspyp gvpveelfkl lkeghrmdkp |
| 721 | snctnelymm mrdcwhavps qrptfkqlve dldrivalts ngeyldlsmp dqyspsfpd |
| 781 | trsstcssge dsvfsheplp eepclprhpa qlangglkrr |

fibroblast growth factor receptor 1 isoform 1 precursor [Homo sapiens]
ACCESSION NP_075598 NP_000595 (SEQ ID NO: 31)

| | |
|---|---|
| 1 | mwswkcllfw avlvtatlct arpsptlpeq aqpwgapvev esflvhpgdl lqlrcrlrdd |
| 61 | vqsinwlrdg vqlaesnrtr itgeevevqd svpadsglya cvtsspsgsd ttyfsvnvsd |
| 121 | alpssedddd dddssseeke tdntkpnrmp vapywtspek mekklhavpa aktvkfkcps |
| 181 | sgtpnptlrw lkngkefkpd hriggykvry atwsiimdsv vpsdkgnytc iveneygsin |
| 241 | htyqldvver sphrpilqag lpanktvalg snvefmckvy sdpqphiqwl khievngski |
| 301 | gpdnlpyvqi lktagvnttd kemevlhirn vsfedageyt clagnsigls hhsawltvle |
| 361 | aleerpavmt splyleiiiy ctgafliscm vgsvivykmk sgtkksdfhs qmavhklaks |
| 421 | iplrrqvtvs adssasmnsg vllvrpsrls ssgtpmlagv seyelpedpr welprdrlvl |
| 481 | gkplgegcfg qvvlaeaigl dkdkpnrvtk vavkmlksda tekdlsdlis ememmkmigk |
| 541 | hkniinllga ctqdgplyvi veyaskgnlr eylqarrppg leycynpshn peeqlsskdl |
| 601 | vscayqvarg meylaskkci hrdlaarnvl vtednvmkia dfglardihh idyykkttng |
| 661 | rlpvkwmape alfdriythq sdvwsfgvll weiftlggsp ypgvpveelf kllkeghrmd |
| 721 | kpsnctnely mmmrdcwhav psqrptfkql vedldrival tsnqeyldls mpldqyspsf |
| 781 | pdtrsstcss gedsvfshep lpeepclprh paqlangglk rr |

fibroblast growth factor receptor 1 isoform 3 precursor [Homo sapiens]
ACCESSION NP_001167537 (SEQ ID NO: 32)

| | |
|---|---|
| 1 | mwswkcllfw avlvtatlct arpsptlpeq dalpsseddd ddddssseek etdntkpnrm |
| 61 | pvapywtspe kmekklhavp aaktvkfkcp ssgtpnptlr wlkngkefkp dhriggykvr |
| 121 | yatwsiimds vvpsdkgnyt civeneygsi nhtyqldvve rsphrpilqa glpanktval |
| 181 | gsnvefmckv ysdpqphiqw lkhievngsk igpdnlpyvq ilktagvntt dkemevlhlr |
| 241 | nvsfedagey tclagnsigl shhsawltvl ealeerpavm tsplyleiii yctgaflisc |
| 301 | mvgsvivykm ksgtkksdfh sqmavhklak siplrrqvtv sadssasmns gvllvrpsrl |
| 361 | sssgtpmlag vseyelpedp rwelprdrlv lgkplgegcf gqvvlaeaig ldkdkpnrvt |
| 421 | kvavkmlksd atekdlsdli sememmkmig khkniinllg actqdgplyv iveyaskgnl |
| 481 | reylqarrpp gleycynpsh npeeqlsskd lvscayqvar gmeylaskkc ihrdlaarnv |
| 541 | lvtednvmki adfglardih hidyykkttn grlpvkwmap ealfdriyth qsdvwsfgvl |
| 601 | lweiftlggs pypgvpveel fkllkeghrm dkpsnctnel ymmmrdcwha vpsqrptfkq |
| 661 | lvedldriva ltsnqeyldl smpldqysps fpdtrsstcs sgedsvfshe plpeepclpr |
| 721 | hpaqlanqql krr |

fibroblast growth factor receptor 1 isoform 4 precursor [Homo sapiens]
ACCESSION NP_075594 (SEQ ID NO: 33)

| | |
|---|---|
| 1 | mwswkcllfw avlvtatlct arpsptlpeq dalpsseddd ddddssseek etdntkpnpv |
| 61 | apywtspekm ekklhavpaa ktvkfkcpss gtpnptlrwl kngkefkpdh riggykvrya |
| 121 | twsiimdsvv psdkgnytci veneygsinh tyqldvvers phrpilqagl panktvalgs |
| 181 | nvefmckvys dpqphiqwlk hievngskig pdnlpyvqil ktagvnttdk emevlhlrnv |
| 241 | sfedageytc lagnsiglsh hsawltvlea leerpavmts plyleiiiyc tgafliscmv |
| 301 | gsvivykmks gtkksdfhsq mavhklaksi plrrqvtvsa dssasmnsgv llvrpsrlss |
| 361 | sgtpmlagvs eyelpedprw elprdrlvlg kplgegcfgq vvlaeaigld kdkpnrvtkv |
| 421 | avkmlksdat ekdlsdlise memmkmigkh kniinllgac tqdgplyviv eyaskgnlre |
| 481 | ylqarrppgl eycynpshnp eeqlsskdlv scayqvargm eylaskkcih rdlaarnvlv |
| 541 | tednvmkiad fglardihhi dyykkttngr lpvkwmapea lfdriythqs dvwsfgvllw |
| 601 | eiftlggspy pgvpveelfk llkeghrmdk psnctnelym mmrdcwhavp sqrptfkqlv |
| 661 | edldrivalt snqeyldlsm pldqyspsfp dtrsstcssg edsvfshepl peepclprhp |
| 721 | aqlangglkr r |

fibroblast growth factor receptor 1 isoform 5, partial [Homo sapiens]
ACCESSION ALQ33527 (SEQ ID NO: 34)

| | |
|---|---|
| 1 | mwswkcllfw avlvtatlct arpsptlpeq dalpsseddd ddddssseek etdntkpnrm |
| 61 | pvapywtspe kmekklhavp aaktvkfkcp ssgtpnptlr wlkngkefkp dhriggykvr |
| 121 | yatwsiimds vvpsdkgnyt civeneygsi nhtyqldvve rsphrpilqa glpanktval |
| 181 | gsnvefmckv ysdpqphiqw lkhievngsk igpdnlpyvq ilktagvntt dkemevlhlr |
| 241 | nvsfedagey tclagnsigl shhsawltvl ealeerpavm tsplyleiii yctgaflisc |
| 301 | mvgsvivykm ksgtkksdfh sqmavhklak siplrrqvsa dssasmnsgv llvrpsrlss |
| 361 | sgtpmlagvs eyelpedprw elprdrlvlg kplgegcfgq vvlaeaigld kdkpnrvtkv |
| 421 | avkmlksdat ekdlsdlise memmkmigkh kniinllgac tqdgplyviv eyaskgnlre |
| 481 | ylqarrppgl eycynpshnp eeqlsskdlv scayqvargm eylaskkcih rdlaarnvlv |
| 541 | tednvmkiad fglardihhi dyykkttngr lpvkwmapea lfdriythqs dvwsfgvllw |
| 601 | eiftlggspy pgvpveelfk llkeghrmdk psnctnelym mmrdcwhavp sqrptfkqlv |
| 661 | edldrivalt snqeyldlsm pldqyspsfp dtrsstcssg edsvfshepl peepclprhp |
| 721 | aqlangglkr r |

fibroblast growth factor receptor 1 isoform 6, partial [Homo sapiens]
ACCESSION ALQ33528 (SEQ ID NO: 35)

| | |
|---|---|
| 1 | mwswkcllfw avlvtatlct arpsptlpeq dalpsseddd ddddssseek etdntkpnpv |
| 61 | apywtspekm ekklhavpaa ktvkfkcpss gtpnptlrwl kngkefkpdh riggykvrya |
| 121 | twsiimdsvv psdkgnytci veneygsinh tyqldvvers phrpilqagl panktvalgs |
| 181 | nvefmckvys dpqphiqwlk hievngskig pdnlpyvqil ktagvnttdk emevlhlrnv |
| 241 | sfedageytc lagnsiglsh hsawltvlea leerpavmts plyleiiiyc tgafliscmv |
| 301 | gsvivykmks gtkksdfhsq mavhklaksi plrrqvsads sasmnsgvll vrpsrlsssg |
| 361 | tpmlagvsey elpedprwel prdrlvlgkp lgegcfgqvv laeaigldkd kpnrvtkvav |
| 421 | kmlksdatek dlsdliseme mmkmigkhkn iinllgactq dgplyvivey askgnlreyl |
| 481 | garrppgley cynpshnpee qlsskdlvsc ayqvargmey laskkcihrd laarnvlvte |
| 541 | dnvmkiadfg lardihhidy ykkttngrlp vkwmapealf driythqsdv wsfgvllwei |
| 601 | ftlggspypg vpveelfkll keghrmdkps nctnelymmm rdcwhavpsq rptfkqlved |
| 661 | ldrivaltsn qeyldlsmpl dqyspsfpdt rsstcssged svfsheplpe epclprhpaq |
| 721 | langglkrr |

fibroblast growth factor receptor 1 isoform 10 precursor [Homo sapiens]
ACCESSION NP_001167534 (SEQ ID NO: 36)

| | |
|---|---|
| 1 | mwswkcllfw avlvtatlct arpsptlpeq aqpwgapvev esflvhpgdl lqlrcrlrdd |
| 61 | vqsinwlrdg vqlaesnrtr itgeevevqd svpadsglya cvtsspsgsd ttyfsvnvsd |
| 121 | alpssedddd dddssseeke tdntkpnrmp vapywtspek mekklhavpa aktvkfkcps |
| 181 | sgtpnptlrw lkngkefkpd hriggykvry atwsiimdsv vpsdkgnytc iveneygsin |
| 241 | htyqldvver sphrpilqag lpanktvalg snvefmckvy sdpqphiqwl khievngski |
| 301 | gpdnlpyvqi lktagvnttd kemevlhlrn vsfedageyt clagnsigls hhsawltvle |
| 361 | aleerpavmt splyleiiiy ctgafliscm vgsvivykmk sgtkksdfhs qmavhklaks |
| 421 | iplrrqvsad ssasmnsgvl lvrpsrlsss gtpmlagvse yelpedprwe lprdrlvlgk |
| 481 | plgegcfgqv vlaeaigldk dkpnrvtkva vkmlksdate kdlsdlisem emmkmigkhk |
| 541 | niinllgact qdgplyvive yaskgnlrey lqarrppgle ycynpshnpe eqlsskdlvs |
| 601 | cayqvargme ylaskkcihr dlaarnvlvt ednvmkiadf glardihhid yykkttngrl |
| 661 | pvkwmapeal fdriythqsd vwsfgvllwe iftlggspyp gvpveelfkl 1keghrmdkp |
| 721 | snctnelymm mrdcwhavps qrptfkqlve dldrivalts nqeyldlsmp ldqyspsfpd |
| 781 | trsstcssge dsvfsheplp eepclprhpa qlangglkrr |

fibroblast growth factor receptor 1 isoform 11 precursor [Homo sapiens]
NP_001167535 XP_005273506 (SEQ ID NO: 37)

| | |
|---|---|
| 1 | maavtrdfge mllhsgrvlp aeaqpwgapv evesflvhpg dllqlrcrlr ddvqsinwlr |
| 61 | dgvqlaesnr tritgeevev qdsvpadsgl yacvtsspsg sdttyfsvnv sdalpssedd |
| 121 | dddddsssee ketdntkpnr mpvapywtsp ekmekklhav paaktvkfkc pssgtpnptl |
| 181 | rwlkngkefk pdhriggykv ryatwsiimd svvpsdkgny tciveneygs inhtyqldvv |
| 241 | ersphrpilq aglpanktva lgsnvefmck vysdpqphiq wlkhievngs kigpdnlpyv |
| 301 | qilktagvnt tdkemevlhl rnvsfedage ytclagnsig lshhsawltv lealeerpav |
| 361 | mtsplyleii iyctgaflis cmvgsvivyk mksgtkksdf hsqmavhkla ksiplrrqvs |
| 421 | adssasmnsg vllvrpsrls ssgtpmlagv seyelpedpr welprdrlvl gkplgegcfg |
| 481 | qvvlaeaigl dkdkpnrvtk vavkmlksda tekdlsdlis ememmkmigk hkniinllga |
| 541 | ctqdgplyvi veyaskgnlr eylqarrppg leycynpshn peeqlsskdl vscayqvarg |
| 601 | meylaskkci hrdlaarnvl vtednvmkia dfglardihh idyykkttng rlpvkwmape |
| 661 | alfdriythq sdvwsfgvll weiftlggsp ypgvpveelf kllkeghrmd kpsnctnely |
| 721 | mmmrdcwhav psqrptfkql vedldrival tsnqeyldls mpldqyspsf pdtrsstcss |
| 781 | gedsvfshep lpeepclprh paqlangglk rr |

fibroblast growth factor receptor 1 isoform 14 precursor [Homo sapiens]
ACCESSION NP_001167538 (SEQ ID NO: 38)

| | |
|---|---|
| 1 | mearvslkrr ieltveypwr cgalsptsnc rtgmwswkcl lfwavlvtat lctarpsptl |
| 61 | peqaqpwgap vevesflvhp gdllqlrcrl rddvqsinwl rdgvqlaesn rtritgeeve |
| 121 | vqdsvpadsg lyacvtssps gsdttyfsvn vsdalpssed ddddddssse eketdntkpn |
| 181 | pvapywtspe kmekklhavp aaktvkfkcp ssgtpnptlr wlkngkefkp dhriggykvr |
| 241 | yatwsiimds vvpsdkgnyt civeneygsi nhtyqldvve rsphrpilqa glpanktval |
| 301 | gsnvefmckv ysdpqphiqw lkhievngsk igpdnlpyvq ilktagvntt dkemevlhlr |
| 361 | nvsfedagey tclagnsigl shhsawltvl ealeerpavm tsplyleiii yctgaflisc |
| 421 | mvgsvivykm ksgtkksdfh sqmavhklak siplrrqvtv sadssasmns gvllvrpsrl |
| 481 | sssgtpmlag vseyelpedp rwelprdrlv lgkplgegcf gqvvlaeaig 1dkdkpnrvt |
| 541 | kvavkmlksd atekdlsdli sememmkmig khkniinllg actqdgplyv iveyaskgnl |
| 601 | reylqarrpp gleycynpsh npeeqlsskd lvscayqvar gmeylaskkc ihrdlaarnv |
| 661 | lvtednvmki adfglardih hidyykkttn grlpvkwmap ealfdriyth qsdvwsfgvl |
| 721 | lweiftlggs pypgvpveel fkllkeghrm dkpsnctnel ymmmrdcwha vpsqrptfkq |
| 781 | lvedldriva ltsnqeyldl smpldqysps fpdtrsstcs sgedsvfshe plpeepclpr |
| 841 | hpaqlanggl krr |

fibroblast growth factor receptor 1 isoform 15 precursor [Homo sapiens]
ACCESSION NP_001341296 XP_006716369 (SEQ ID NO: 39)

| | |
|---|---|
| 1 | mwswkcllfw avlvtatlct arpsptlpeq aqpwgapvev esflvhpgdl lqlrcrlrdd |
| 61 | vqsinwlrdg vqlaesnrtr itgeevevqd svpadsglya cvtsspsgsd ttyfsvnvsd |
| 121 | alpssedddd dddssseeke tdntkpnpva pywtspekme kklhavpaak tvkfkcpssg |
| 181 | tpnptlrwlk ngkefkpdhr iggykvryat wsiimdsvvp sdkgnytciv eneygsinht |
| 241 | yqldvversp hrpilqaglp anktvalgsn vefmckvysd pqphiqwlkh ievngskigp |
| 301 | dnlpyvqilk tagvnttdke mevlhlrnvs fedageytcl agnsiglshh sawltvleal |
| 361 | eerpavmtsp lyleiiiyct gafliscmvg svivykmksg tkksdfhsqm avhklaksip |
| 421 | lrrqvtvsad ssasmnsgvl lvrpsrlsss gtpmlagvse yelpedprwe lprdrlvlgk |
| 481 | plgegcfgqv vlaeaigldk dkpnrvtkva vkmlksdate kdlsdlisem emmkmigkhk |
| 541 | niinllgact qdgplyvive yaskgnlrey lqarrppgle ycynpshnpe eqlsskdlvs |
| 601 | cayqvargme ylaskkcihr dlaarnvlvt ednvmkiadf glardihhid yykkttngrl |
| 661 | pvkwmapeal fdriythqsd vwsfgvllwe iftlggspyp gvpveelfkl 1keghrmdkp |
| 721 | snctnelymm mrdcwhavps qrptfkqlve dldrivalts nqvlhpdltt hllplrcfwk |
| 781 | vrkrrcllrg qephpcsrga gqrvkaqqps tmdgflqgnr wgwagegapt |

fibroblast growth factor receptor 1 isoform 16 precursor [Homo sapiens]
ACCESSION NP_001341297 XP_016868717 (SEQ ID NO: 40)

| | |
|---|---|
| 1 | mwswkcllfw avlvtatlct arpsptlpeq dalpsseddd ddddssseek etdntkpnpv |
| 61 | apywtspekm ekklhavpaa ktvkfkcpss gtpnptlrwl kngkefkpdh riggykvrya |
| 121 | twsiimdsvv psdkgnytci veneygsinh tyqldvvers phrpilqagl panktvalgs |
| 181 | nvefmckvys dpqphiqwlk hievngskig pdnlpyvqil ktagvnttdk emevlhlrnv |
| 241 | sfedageytc lagnsiglsh hsawltvlea leerpavmts plyleiiiyc tgafliscmv |
| 301 | gsvivykmks gtkksdfhsq mavhklaksi plrrqvsads sasmnsgvll vrpsrlsssg |
| 361 | tpmlagvsey elpedprwel prdrlvlgkp lgegcfgqvv laeaigldkd kpnrvtkvav |
| 421 | kmlksdatek dlsdliseme mmkmigkhkn iinllgactq dgplyvivey askgnlreyl |
| 481 | qarrppgley cynpshnpee qlsskdlvsc ayqvargmey laskkcihrd laarnvlvte |
| 541 | dnvmkiadfg lardihhidy ykkttngrlp vkwmapealf driythqsdv wsfgvllwei |
| 601 | ftlggspypg vpveelfkll keghrmdkps nctnelymmm rdcwhavpsq rptfkqlved |
| 661 | ldrivaltsn qeyldlsmpl dqyspsfpdt rsstcssged svfsheplpe epclprhpaq |
| 721 | langglkrr |

fibroblast growth factor receptor 1 isoform 17 precursor [Homo sapiens]
ACCESSION NP_001341298 XP_016868712 (SEQ ID NO: 41)

| | |
|---|---|
| 1 | mwswkcllfw avlvtatlct arpsptlpeq aqpwgapvev esflvhpgdl lqlrcrlrdd |
| 61 | vqsinwlrdg vqlaesnrtr itgeevevqd svpadsglya cvtsspsgsd ttyfsvnvsd |
| 121 | alpssedddd dddssseeke tdntkpnpva pywtspekme kklhavpaak tvkfkcpssg |
| 181 | tpnptlrwlk ngkefkpdhr iggykvryat wsiimdsvvp sdkgnytciv eneygsinht |
| 241 | yqldvversp hrpilqaglp anktvalgsn vefmckvysd pqphiqwlkh ievngskigp |
| 301 | dnlpyvqilk tagvnttdke mevlhlrnvs fedageytcl agnsiglshh sawltvleal |
| 361 | eerpavmtsp lyleiiiyct gafliscmvg svivykmksg tkksdfhsqm avhklaksip |
| 421 | lrrqvsadss asmnsgvllv rpsrlsssgt pmlagvseye lpedprwelp rdrlvlgkpl |
| 481 | gegcfgqvvl aeaigldkdk pnrvtkvavk mlksdatekd lsdlisemem mkmigkhkni |
| 541 | inllgactqd gplyviveya skgnlreylq arrppgleyc ynpshnpeeq lsskdlvsca |
| 601 | yqvargmeyl askkcihrdl aarnvlvted nvmkiadfgl ardihhidyy kkttngrlpv |
| 661 | kwmapealfd riythqsdvw sfgvllweif tlggspypgv pveelfkllk eghrmdkpsn |
| 721 | ctnelymmmr dcwhavpsqr ptfkqlvedl drivaltsnq vlhpdltthl lplrcfwkvr |
| 781 | krrcllrgqe phpcsrgagq rvkaqqpstm dgflqgnrwg wagegapt |

fibroblast growth factor receptor 1 isoform 18 precursor [Homo sapiens]
ACCESSION NP_001341299 XP_006716376 (SEQ ID NO: 42)

| | |
|---|---|
| 1 | mwswkcllfw avlvtatlct arpsptlpeq dalpsseddd ddddssseek etdntkpnpv |
| 61 | apywtspekm ekklhavpaa ktvkfkcpss gtpnptlrwl kngkefkpdh riggykvrya |
| 121 | twsiimdsvv psdkgnytci veneygsinh tyqldvvers phrpilqagl panktvalgs |
| 181 | nvefmckvys dpqphiqwlk hievngskig pdnlpyvqil ktagvnttdk emevlhlrnv |
| 241 | sfedageytc lagnsiglsh hsawltvlea leerpavmts plyleiiiyc tgafliscmv |
| 301 | gsvivykmks gtkksdfhsq mavhklaksi plrrqvtvsa dssasmnsgv llvrpsrlss |
| 361 | sgtpmlagvs eyelpedprw elprdrlvlg kplgegcfgq vvlaeaigld kdkpnrvtkv |
| 421 | avkmlksdat ekdlsdlise memmkmigkh kniinllgac tqdgplyviv eyaskgnlre |
| 481 | ylqarrppgl eycynpshnp eeqlsskdlv scayqvargm eylaskkcih rdlaarnvlv |
| 541 | tednvmkiad fglardihhi dyykkttngr lpvkwmapea lfdriythqs dvwsfgvllw |
| 601 | eiftlggspy pgvpveelfk llkeghrmdk psnctnelym mmrdcwhavp sqrptfkqlv |
| 661 | edldrivalt snqvlhpdlt thllplrcfw kvrkrrcllr gqephpcsrg agqrvkaqqp |
| 721 | stmdgflqgn rwgwagegap t |

19. Jag1
Jagged1 [Homo sapiens]
ACCESSION AAC51731 NP_000205 EAX10341 AAI26208 AAI26206 AAH98393 (SEQ ID NO: 43)

| | |
|---|---|
| 1 | mrsprtrgrs grplslllal lcalrakvcg asgqfeleil smqnvngelq ngnccggarn |
| 61 | pgdrkctrde cdtyfkvolk eyqsrvtagg pcsfgsgstp viggntfnlk asrgndrnri |
| 121 | vlpfsfawpr sytllveawd ssndtvqpds iiekashsgm inpsrqwqtl kqntgvahfe |
| 181 | yqirvtcddy yygfgenkfc rprddffghy acdqngnktc megwmgreen raicrqgcsp |
| 241 | khgscklpgd crcqygwqgl ycdkciphpg cvhgicnepw qclcetnwgg qlcdkdlnyc |
| 301 | gthqpclngg tcsntgpdky qcscpegysg pnceiaehac lsdpchnrgs cketslgfec |
| 361 | ecspgwtgpt cstniddcsp nncshggtcq dlvngfkcvc ppqwtgktcq ldaneceakp |
| 421 | cvnakscknl iasyycdclp gwmgqncdin indclgqcqn dascrdlvng yrcicppgya |
| 481 | gdhcerdide casnpcldgg hcqneinrfq clcptgfsgn lcqldidyce pnpcqngaqc |
| 541 | ynrasdyfck cpedyegknc shlkdhcrtt pcevidsctv amasndtpeg vryissnvcg |
| 601 | phgkcksqsg gkftcdonkg ftgtycheni ndcesnporn ggtcidgvns ykcicsdgwe |
| 661 | gaycetnind csqnpchngg tcrdlvndfy cdckngwkgk tchsrdsqcd eatonnggtc |
| 721 | ydegdafkcm cpggwegttc niarnssclp npchnggtcv vngesftcvc kegwegpica |
| 781 | qntndcsphp cynsgtcvdg dnwyrcecap gfagpdcrin inecqsspca fgatcvdein |
| 841 | gyrcvcppgh sgakcqevsg rpcitmgsvi pdgakwdddc ntcqclngri acskvwcgpr |
| 901 | pcllhkghse cpsgqscipi lddqcfvhpc tgvgecrsss lqpvktkcts dsyyqdncan |
| 961 | itftfnkemm spglttehic selrnlnilk nvsaeysiyi acepspsann eihvaisaed |
| 1021 | irddgnpike itdkiidlvs krdgnsslia avaevrvqrr plknrtdflv pllssvltva |
| 1081 | wicclvtafy wclrkrrkpg shthsasedn ttnnvreqln qiknpiekhg antvpikdye |
| 1141 | nknskmskir thnseveedd mdkhqqkarf akqpaytlvd reekppngtp tkhpnwtnkq |
| 1201 | dnrdlesaqs lnrmeyiv |

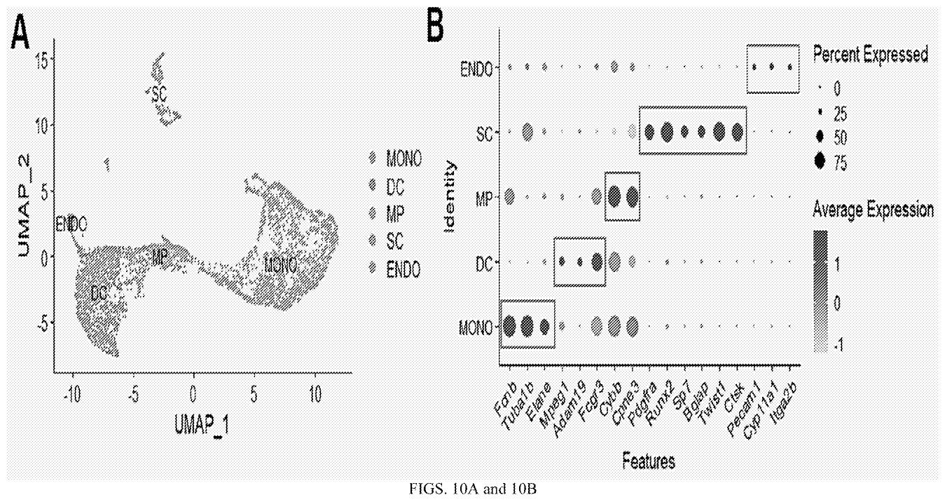

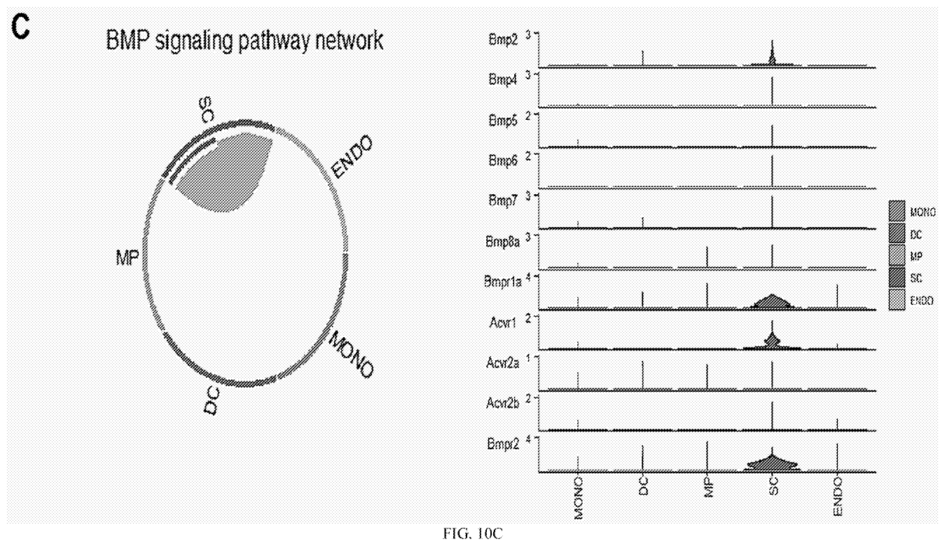

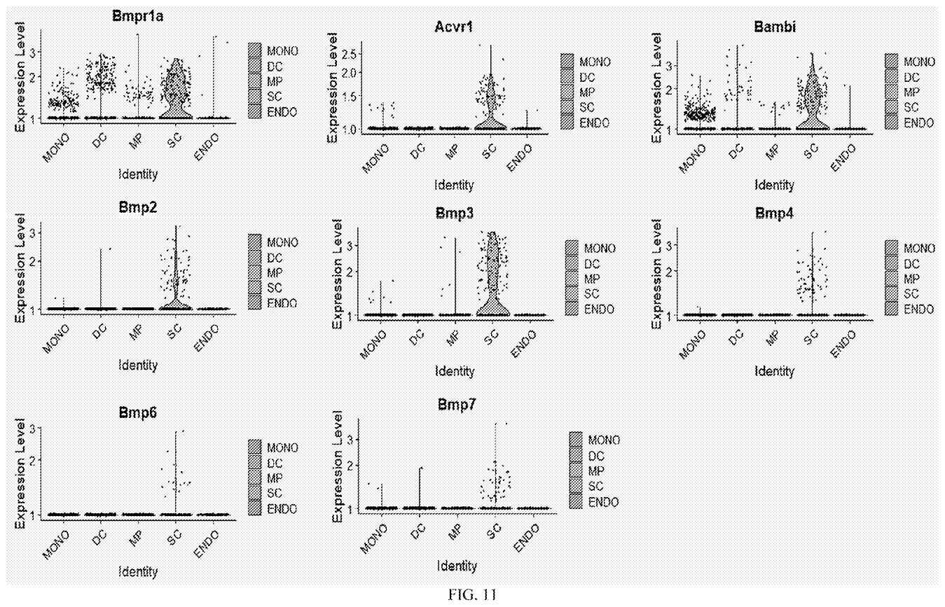

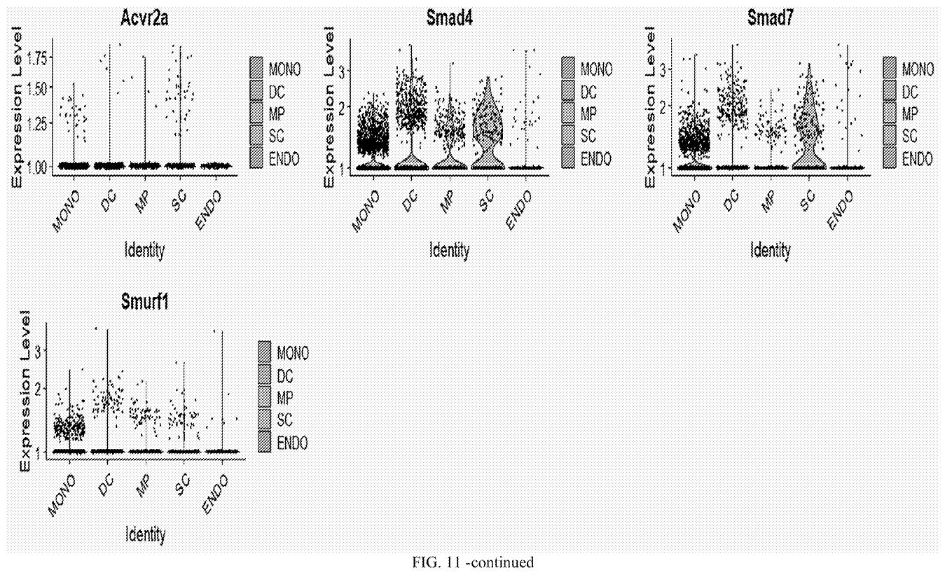

Group 3 From FIG. 10 & 11 (BMP signaling)
20. Acvr1 (SEQ ID NO: 44)

| | |
|---|---|
| 1 | mvdgvmilpv limialpsps medekpkvnp klymcvcegl scgnedhceg qqcfsslsin |
| 61 | dgfhvyqkgc fqvyeqgkmt cktppspgqa veccqgdwon rnitaqlptk gksfpgtqnf |
| 121 | hlevgliils vvfavcllac llgvalrkfk ringerlnpr dveygtiegl ittnvgdstl |
| 181 | adlldhscts gsgsglpflv qrtvarqitl lecvgkgryg evwrgswqge nvavkifssr |
| 241 | dekswyrete lyntvmlrhe nilgfiasdm tsrhsstqlw lithyhemgs lydylqlttl |
| 301 | dtvsclrivl siasglahlh ieifgtqgkp aiahrdlksk nilvkkngqc ciadlglavm |
| 361 | hsqstnqldv gnnprvgtkr ymapevldet iqvdcfdsyk rvdiwafglv lwevarrmvs |
| 421 | ngivedykpp fydvvpndps fedmrkvvcv dqqrpnipnr wfsdptltsl aklmkecwyq |
| 481 | npsarltalr ikktltkidn sldklktdc |

21. Acvr2a (SEQ ID NO: 45)

| | |
|---|---|
| 1 | mcnekfsyfp emevtqptsn pvtpkppyyn illyslvplm liagivicaf wvyrhhkmay |
| 61 | ppvlvptqdp gppppspllg lkplqllevk argrfgcvwk aqllneyvav kifpiqdkqs |
| 121 | wqneyevysl pgmkhenilq figaekrgts vdvdlwlita fhekgslsdf 1kanvvswne |
| 181 | lchiaetmar glaylhedip glkdghkpai shrdiksknv llknnltaci adfglalkfe |
| 241 | agksagdthg qvgtrrymap evlegainfq rdaflridmy amglvlwela srctaadgpv |
| 301 | deymlpfeee igqhpsledm qevvvhkkkr pvlrdywqkh agmamlceti eecwdhdaea |
| 361 | rlsagcvger itqmqrltni ittedivtvv tmvtnvdfpp kessl |

22. Acvr2b (SEQ ID NO: 46)

| | |
|---|---|
| 1 | mndfvavkif plqdkqswqs ereifstpgm khenllqfia aekrgsnlev elwlitafhd |
| 61 | kgsltdylkg niitwnelch vaetmsrgls ylhedvpwcr geghkpsiah rdfksknvll |
| 121 | ksdltavlad fglavrfepg kppgdthgqv gtrrymapev legainfqrd aflridmyam |
| 181 | glvlwelvsr ckaadgpvde ymlpfeeeig qhpsleelqe vvvhkkmrpt ikdhwlkhpg |
| 241 | laqlcvtiee cwdhdaearl sagcveervs lirrsvngtt sdclvslvts vtnvdlppke |
| 301 | ssi |

23. Bmpr2 (see above)
Group 4 Stem Cell Markers But Not Cell Surface Markers
24. Erg
transcriptional regulator ERG isoform 1 [Homo sapiens]
ACCESSION NP_891548 (SEQ ID NO: 47)

| | |
|---|---|
| 1 | mastikeals vvsedqslfe caygtphlak temtassssd ygqtskmspr vpqqdwlsqp |
| 61 | parvtikmec npsqvngsrn spdecsvakg gkmvgspdtv gmnygsymee khmpppnmtt |
| 121 | nerrvivpad ptlwstdhvr qwlewavkey glpdvnillf qnidgkelck mtkddfqrlt |
| 181 | psynadills hlhylretpl phltsddvdk alqnsprlmh arntggaafi fpntsvypea |
| 241 | tqrittrpdl pyepprrsaw tghghptpqs kaaqpspstv pktedqrpql dpyqilgpts |
| 301 | srlanpgsgq iqlwqfllel lsdssnssci twegtngefk mtdpdevarr wgerkskpnm |
| 361 | nydklsralr yyydknimtk vhgkryaykf dfhgiagalq phppesslyk ypsdlpymgs |
| 421 | yhahpqkmnf vaphppalpv tsssffaapn pywnsptggi ypntriptsh mpshlgtyy |

transcriptional regulator ERG isoform 2 [Homo sapiens]
ACCESSION NP_004440 (SEQ ID NO: 48)

| | |
|---|---|
| 1 | miqtvpdpaa hikealsvvs edqslfecay gtphlaktem tassssdygq tskmsprvpq |
| 61 | qdwlsqppar vtikmecnps qvngsrnspd ecsvakggkm vgspdtvgmn ygsymeekhm |
| 121 | pppnmttner rvivpadptl wstdhvrqwl ewavkeyglp dvnillfqni dgkelckmtk |
| 181 | ddfqrltpsy nadillshlh ylretplphl tsddvdkalq nsprlmharn tdlpyepprr |
| 241 | sawtghghpt pqskaaqpsp stvpktedqr pqldpyqilg ptssrlanpg sgqiqlwqfl |
| 301 | lellsdssns scitwegtng efkmtdpdev arrwgerksk pnmnydklsr alryyydkni |
| 361 | mtkvhgkrya ykfdfhgiaq alqphppess lykypsdlpy mgsyhahpqk mnfvaphppa |
| 421 | lpvtsssffa apnpywnspt ggiypntrlp tshmpshlgt yy |

transcriptional regulator ERG isoform 3 [Homo sapiens]
ACCESSION NP_001129626 (SEQ ID NO: 49)

| | |
|---|---|
| 1 | miqtvpdpaa hikealsvvs edqslfecay gtphlaktem tassssdygq tskmsprvpq |
| 61 | qdwlsqppar vtikmecnps qvngsrnspd ecsvakggkm vgspdtvgmn ygsymeekhm |
| 121 | pppnmttner rvivpadptl wstdhvrqwl ewavkeyglp dvnillfqni dgkelckmtk |
| 181 | ddfqrltpsy nadillshlh ylretplphl tsddvdkalq nsprlmharn tggaafifpn |
| 241 | tsvypeatqr ittrpdlpye pprrsawtgh ghptpqskaa qpspstvpkt edqrpqldpy |
| 301 | qilgptssrl anpgsgqiql wqfllellsd ssnsscitwe gtngefkmtd pdevarrwge |
| 361 | rkskpnmnyd klsralryyy dknimtkvhg kryaykfdfh giaqalqphp pesslykyps |
| 421 | dlpymgsyha hpqkmnfvap hppalpvtss sffaapnpyw nsptggiypn trlptshmps |
| 481 | hlgtyy |

transcriptional regulator ERG isoform 4 [Homo sapiens]
ACCESSION NP_001129627 (SEQ ID NO: 50)

| | |
|---|---|
| 1 | mvgspdtvgm nygsymeekh mpppnmttne rrvivpadpt lwstdhvrqw lewavkeygl |
| 61 | pdvnillfqn idgkelckmt kddfqrltps ynadillshl hylretplph ltsddvdkal |
| 121 | qnsprlmhar ntggaafifp ntsvypeatq rittrpdlpy epprrsawtg hghptpqska |
| 181 | aqpspstvpk tedqrpqldp yqilgptssr lanpgsgqiq lwqfllells dssnsscitw |
| 241 | egtngefkmt dpdevarrwg erkskpnmny dklsralryy ydknimtkvh gkryaykfdf |
| 301 | hgiaqalqph ppesslykyp sdlpymgsyh ahpqkmnfva phppalpvts ssffaapnpy |
| 361 | wnsptggiyp ntrlptshmp shlgtyy |

transcriptional regulator ERG isoform 5 [Homo sapiens]
ACCESSION NP_001230358 (SEQ ID NO: 51)

| | |
|---|---|
| 1 | mvgspdtvgm nygsymeekh mpppnmttne rrvivpadpt lwstdhvrqw lewavkeygl |
| 61 | pdvnillfqn idgkelckmt kddfqrltps ynadillshl hylretplph ltsddvdkal |
| 121 | qnsprlmhar ntdlpyeppr rsawtghghp tpqskaagps pstvpktedq rpqldpyqil |
| 181 | gptssrlanp gsgqiqlwqf llellsdssn sscitwegtn gefkmtdpde varrwgerks |
| 241 | kpnmnydkls ralryyydkn imtkvhgkry aykfdfhgia qalqphppes slykypsdlp |
| 301 | ymgsyhahpq kmnfvaphpp alpvtsssff aapnpywnsp tggiypntrl ptshmpshlg |
| 361 | tyy |

transcriptional regulator ERG isoform 6 [Homo sapiens]
ACCESSION NP_001230361 (SEQ ID NO: 52)

| | |
|---|---|
| 1 | miqtvpdpaa hikealsvvs edqslfecay gtphlaktem tassssdygq tskmsprvpq |
| 61 | qdwlsqppar vtikmecnps qvngsrnspd ecsvakggkm vgspdtvgmn ygsymeekhm |
| 121 | pppnmttner rvivpadptl wstdhvrqwl ewavkeyglp dvnillfqni dgkelckmtk |
| 181 | ddfqrltpsy nadillshlh ylretplphl tsddvdkalq nsprlmharn tggaafifpn |
| 241 | tsvypeatqr ittrpdlpye pprrsawtgh ghptpqskaa qpspstvpkt edqrpqldpy |
| 301 | qilgptssrl anpgwtq |

transcriptional regulator ERG isoform 7 [Homo sapiens]
ACCESSION NP_001278320 (SEQ ID NO: 53)

| | |
|---|---|
| 1 | miqtvpdpaa hikealsvvs edqslfecay gtphlaktem tassssdygq tskmsprvpq |
| 61 | qdwlsqppar vtikmecnps qvngsrnspd ecsvakggkm vgspdtvgmn ygsymeekhm |
| 121 | pppnmttner rvivpadptl wstdhvrqwl ewavkeyglp dvnillfqni dgkelckmtk |
| 181 | ddfqrltpsy nadillshlh ylretplphl tsddvdkalq nsprlmharn tggaafifpn |
| 241 | tsvypeatqr ittrpgtktp lcdlfierhp rcpaeirals hviqrelipe lkpvpdslil |
| 301 | plliwrlnpl kpfhskttlk elrad |

transcriptional regulator ERG isoform 8 [Homo sapiens]
ACCESSION NP_001317954 XP_016883777 (SEQ ID NO: 54)

| | |
|---|---|
| 1 | mastikeals vvsedqslfe caygtphlak temtassssd ygqtskmspr vpqqdwlsqp |
| 61 | parvtikmec npsqvngsrn spdecsvakg gkmvgspdtv gmnygsymee khmpppnmtt |
| 121 | nerrvivpad ptlwstdhvr qwlewavkey glpdvnillf qnidgkelck mtkddfqrlt |
| 181 | psynadills hlhylretpl phltsddvdk alqnsprlmh arntdlpyep prrsawtghg |
| 241 | hptpqskaaq pspstvpkte dqrpqldpyq ilgptssrla npgsgqiqlw qfllellsds |
| 301 | snsscitweg tngefkmtdp devarrwger kskpnmnydk lsralryyyd knimtkvhgk |
| 361 | ryaykfdfhg iaqalqphpp esslykypsd lpymgsyhah pqkmnfvaph ppalpvtsss |
| 421 | ffaapnpywn sptggiypnt rlptshmpsh lgtyy |

25. Six2
SIX2 [Homo sapiens]
ACCESSION AAK16583 (SEQ ID NO: 55)

| | |
|---|---|
| 1 | msmlptfgft qeqvacvcev lqqggnierl grflwslpac ehlhknesvl kakavvafhr |
| 61 | iwdgeetsyc fkeksrsvlr ewyahnpyps prekrelaea tgltttqvsn wfknrrqrdr |
| 121 | gnfrelykil eshqfsphnh aklqqlwlka hyieaeklrg rplgavgkyr vrrkfplprs |
| 181 | aaeakerenn ensnsnshnp lngsgksvlg ssedektpsg tpdhsssspa lllsppppgl |
| 241 | pslhslghpp gpsavpvpvp ggggadplqh hhglqdsiln pmsanlvdlg s |

homeobox protein SIX2 isoform X1 [Homo sapiens]
ACCESSION XP_005264157 (SEQ ID NO: 56)

| | |
|---|---|
| 1 | msmlptfgft qeqvacvcev lqqggnierl grflwslpac ehlhknesvl kakavvafhr |
| 61 | gnfrelykil eshqfsphnh aklqqlwlka hyieaeklrg rplgavgkyr vrrkfplprs |
| 121 | iwdgeetsyc fkeksrsvlr ewyahnpyps prekrelaea tgltttqvsn wfknrrqrdr |
| 181 | aaeakeryee nnensnsnsh nplngsgksv lgssedektp sgtpdhssss palllspppp |
| 241 | glpslhslgh ppgpsavpvp vpggggadpl qhhhglqdsi lnpmsanlvd lgs |

26. Pthlh
parathyroid hormone-related protein isoform 1 preproprotein [Homo sapiens],
ACCESSION NP_945317.1 (SEQ ID NO: 57)

| | |
|---|---|
| 1 | mqrrlvqqws vavfllsyav pscgrsvegl srrlkravse hqllhdkgks iqdlrrrffl |
| 61 | hhliaeihta eiratsevsp nskpspntkn hpvrfgsdde gryltqetnk vetykeqplk |
| 121 | tpgkkkkgkp gkrkegekkk rrtrsawlds gvtgsglegd hlsdtsttsl eldsrrh |

parathyroid hormone-related protein isoform 2 preproprotein [Homo sapiens] ,
ACCESSION NP_945315 (SEQ ID NO: 58)

| | |
|---|---|
| 1 | mqrrlvqqws vavfllsyav pscgrsvegl srrlkravse hqllhdkgks iqdlrrrffl |
| 61 | hhliaeihta eiratsevsp nskpspntkn hpvrfgsdde gryltqetnk vetykeqplk |
| 121 | tpgkkkkgkp gkrkegekkk rrtrsawlds gvtgsglegd hlsdtsttsl eldsr |

27. Twist1
twist-related protein 1 [Homo sapiens]
ACCESSION NP_000465 (SEQ ID NO: 59)

| | |
|---|---|
| 1 | mmqdvssspv spaddslsns eeepdrqqpp sgkrggrkrr ssrrsaggga gpggaagggv |
| 61 | gggdepgspa qgkrgkksag cgggggaggg ggsssgggsp qsyeelqtqr vmanvrerqr |
| 121 | tqslneafaa lrkiiptlps dklskiqtlk laaryidfly qvlqsdelds kmascsyvah |
| 181 | erlsyafsvw rmegawsmsa sh |

twist homolog 1 (acrocephalosyndactyly 3; Saethre-Chotzen syndrome)
(Drosophila) [Homo sapiens]
ACCESSION EAW93711 (SEQ ID NO: 60)

| | |
|---|---|
| 1 | mmqdvssspv spaddslsns eeepdrqqpp sgkrggrkrr ssrrsaggga gpggggwgrr |
| 61 | rrrragqpgp gqarqevcgl wrrrrrgrrr rqqqrrresa vlrgaadaag hgqragapah |
| 121 | pvaergvrra aedhphaalg qaeqdsdpqa ggqvhrlplp gpperraglq dgklqlcgsr |
| 181 | aaqlrllgle dggglvhvrv plaggaphpl sragdldviv sregengqsr dsgag |

28. Alpl (SEQ ID NO: 61)

| | |
|---|---|
| 1 | mispflvlai gtcltnslvp ekekdpkywr dqaqetlkya lelqklntnv aknvimflgd |
| 61 | gmgvstvtaa rilkgqlhhn pgeetrlemd kfpfvalskt yntnaqvpds agtataylcg |
| 121 | vkanegtvgv saatersron ttqgnevtsi lrwakdagks vgivtttrvn hatpsaayah |
| 181 | sadrdwysdn emppealsqg ckdiayqlmh nirdidvimg ggrkymypkn ktdveyesde |
| 241 | kargtrldgl dlvdtwksfk prykhshfiw nrtelltldp hnvdyllglf epgdmqyeln |
| 301 | rnnvtdpsls emvvvaiqil rknpkgffll veggridhgh hegkakqalh eavemdraig |
| 361 | qagsltssed tltvvtadhs hvftfggytp rgnsifglap mlsdtdkkpf tailygngpg |
| 421 | ykvvggeren vsmvdyahnn yqaqsavplr hethggedva vfskgpmahl hgvheqnyv |
| 481 | phvmayaaci ganlghcapa ssagslaagp lllalalypl svlf |

29. Msx1 (SEQ ID NO: 62)

| | |
|---|---|
| 1 | mtslplgvkv edsafgkpag ggagqapsaa aataaamgad eegakpkvsp sllpfsveal |
| 61 | madhrkpgak esalapsegv qaaggsaqpl gvppgslgap dapssprplg hfsvggllkl |
| 121 | pedalvkaes pekpertpwm qsprfspppa rrlsppactl rkhktnrkpr tpfttaqlla |
| 181 | lerkfrqkqy lsiaeraefs sslsltetqv kiwfqnrrak akrlqeaele klkmaakpml |
| 241 | ppaafglsfp lggpaavaaa agaslygasg pfqraalpva pvglytahvg ysmyhlt |

30. Efnb1
ephrin-B1 precursor [Homo sapiens]
ACCESSION NP_004420 (SEQ ID NO: 63)

| | |
|---|---|
| 1 | marpgqrwlg kwlvamvvwa lcrlatplak nlepvswssl npkflsgkgl viypkigdkl |
| 61 | diicpraeag rpyeyyklyl vrpeqaaacs tvldpnvlvt cnrpeqeirf tikfqefspn |
| 121 | ymglefkkhh dyyitstsng sleglenreg gvcrtrtmki imkvgqdpna vtpeqlttsr |
| 181 | pskeadntvk matqapgsrg slgdsdgkhe tvngeeksgp gasggssgdp dgffnskval |
| 241 | faavgagcvi flliiifltv lllklrkrhr khtqqraaal slstlaspkg gsgtagteps |
| 301 | diiiplrtte nnycphyekv sgdyghpvyi vqemppqspa niyykv |

31. Zic1
zinc finger protein ZIC 1 [Homo sapiens]
ACCESSION NP_003403 (SEQ ID NO: 64)

| | |
|---|---|
| 1 | mlldagpqyp aigvttfgas rhhsagdvae rdvglginpf adgmgafkln psshelasag |
| 61 | qtaftsqapg yaaaaalghh hhpghvgsys saafnstrdf lfrnrgfgda aaaasaqhsl |
| 121 | faasaggfgg phghtdaagh llfpglhega aghaspnvvn gqmrlgfsgd myprpegygq |
| 181 | vtsprsehya apqlhgygpm nvnmaahhga gaffrymrqp ikqelickwi epeqlanpkk |
| 241 | scnktfstmh elvthvtveh vggpeqsnhi cfweecpreg kpfkakyklv nhirvhtgek |
| 301 | pfpcpfpgcg kvfarsenlk ihkrthtgek pfkcefegcd rrfanssdrk khmhvhtsdk |
| 361 | pylckmcdks ythpsslrkh mkvhesssqg sqpspaassg yesstpptiv spstdnptts |
| 421 | slspsssavh htaghsalss nfnewyv |

32. Spry1
ACCESSION NP_001362339 XP_005262743 (SEQ ID NO: 65)

| | |
|---|---|
| 1 | mdpqnqhgsg sslvviqqps ldsrqrldye reiqptails ldqikairgs neytegpsvv |
| 61 | krpaprtapr qekherthei ipinvnnnye hrhtshlgha vlpsnargpi lsrststgsa |
| 121 | assgsnssas seqgllgrsp ptrpvpghrs erairtqpkq livddlkgsl kedltqhkfi |
| 181 | ceqcgkckcg ectaprtlps clacnrqclc saesmveygt cmclvkgify hcsnddegds |
| 241 | ysdnpcscsq shccsrylcm gamslflpcl lcyppakgcl klcrrcydwi hrpgcrckns |
| 301 | ntvycklesc psrgqgkps |

33. Abcc9 (SEQ ID NO: 66)

| | |
|---|---|
| 1 | mslsfcgnni ssynindgvl qnscfvdaln lvphvfllfi tfpilfigwg sqsskvqihh |
| 61 | ntwlhfpghn lrwiltfall fvhvceiaeg ivsdsrresr hlhlfmpavm gfvatttsiv |
| 121 | yyhnietsnf pklllgfshh eskscyqhs |

34. Erf (SEQ ID NO: 67)

| | |
|---|---|
| 1 | mktpadtgfa fpdwaykpes spgsrqiqlw hfilellrke eyqgviawqg dygefvikdp |
| 61 | devarlwgvr kckpqmnydk lsralryyyn krilhktkgk rftykfnfnk lvlvnypfid |
| 121 | vglaggavpq sappvpsggs hfrfppstps evlsptedpr sppacsssss slfsavvarr |
| 181 | lgrgsvsdcs dgtseleepl gedprarppg ppdlgafrgp plarlphdpg vfrvyprprg |
| 241 | gpeplspfpv splagpgsll ppqlspalpm tpthlaytps ptlspmypsg gggpsgsggg |
| 301 | shfsfspedm krylqahtqs vynyhlspra flhypglvvp qpqrpdkcpl ppmapetppv |
| 361 | pssassssss ssspfkfklq rpplgrrqra agekavaaad ksggsaggla egagalappp |
| 421 | pppqikvepi segeseevev tdisdedeed gevfktprap pappkpepge apgasqcmpl |
| 481 | klrfkrrwse dcrleggggp aggfedeged kkvrgegpge aggpltprrv ssdlqhataq |
| 541 | lslehrds |

35. Bmp2
bone morphogenetic protein 2 [Homo sapiens]
ACCESSION ACV32596 (SEQ ID NO: 68)

| | |
|---|---|
| 1 | mvagtrclla lllpqvllgg aaglvpelgr rkfaaassgr pssqpsdevl sefelrllsm |
| 61 | fglkqrptps rdavvppyml dlyrrhsgqp sspapdhrle raasrantvr sfhheeslee |
| 121 | lpetsgkttr rfffnlssip teefitsael qvfreqmqda lgnnssfhhr iniyeiikpa |
| 181 | tanskfpvtr lldtrlvnqn asrwesfdvt pavmrwtagg hanhgfvvev ahleekqgvs |
| 241 | krhvrisrsl hqdehswsqi rpllvtfghd ekghplhkre nckpntnsen alspavgdpp |
| 301 | llvnfsdvgw ndwivappgy hafychgecp fpladhlnst nhaivqtlvn svnskipkac |
| 361 | cvptelsais mlyldenekv vlknyqdmvv egcgcr |

bone morphogenetic protein 2, isoform CRA a [Homo sapiens]
ACCESSION EAX10386 (SEQ ID NO: 69)

| | |
|---|---|
| 1 | mfglkqrptp srdavvppym ldlyrrhsgq pgspapdhrl eraasrantv rsfhheesle |
| 61 | elpetsgktt rrfffnlssi pteefitsae lqvfreqmqd algnnssfhh riniyeiikp |
| 121 | atanskfpvt slldtrlvnq nasrwesfdv tpavmrwtaq ghanhgfvve vahleekqgv |
| 181 | skrhvrisrs lhqdehswsq irpllvtfgh dgkghplhkr ekrqakhkqr krlkssckrh |
| 241 | plyvdfsdvg wndwivappg yhafychgec pfpladhlns tnhaivqtlv nsvnskipka |
| 301 | ccvptelsai smlyldenek vvlknyqdmv vegcgcr |

36. Bmp3 (SEQ ID NO: 70)

| | |
|---|---|
| 1 | magasrllfl wlgcfcvsla qgerpkppfp elrkavpgdr tagggpdsel qpqdkvsehm |
| 61 | lrlydrystv qaartpgsle ggsqpwrprl lregntvrsf raaaaetler kglyifnlts |
| 121 | ltksenilsa tlyfcigelg nislscpvsg gcshhaqrkh iqidlsawtl kfsrnqsqll |
| 181 | ghlsvdmaks hrdimswlsk ditqllrkak eneefligfn itskgrqlpk rrlpfpepyi |
| 241 | lvyandaais epesvvsslq ghrnfptgtv pkwdshiraa lsierrkkrs tgvllplqnn |
| 301 | elpgaeyqyk kdevweerkp yktlqaqape ksknkkkqrk gphrksqtlq fdeqtlkkar |
| 361 | rkqwieprnc arrylkslkp snhatiqsiv ravgvvpgip epccvpekms slsilffden |
| 421 | knvvlkvypn mtvescacr |

37. Bmp4 (SEQ ID NO: 71)

| | |
|---|---|
| 1 | mregrgggre grsaepgpea rshsvvpsra thccsfpepf qqvcsrlavk nhglllyalf |
| 61 | svillggash aslipetgkk kvaeiqghag grrsgqshel lrdfeatllq mfglrrrpqp |
| 121 | sksavipdym rdlyrlqsge eeeeqihstg leyperpasr antvrsfhhe ehlenipgts |
| 181 | ensafrflfn lssipenevi ssaelrlfre qvdqgpdwer gfhriniyev mkppaevvpg |
| 241 | hlitrlldtr lvhhnvtrwe tfdvspavlr wtrekqpnyg laievthlhq trthqgqhvr |
| 301 | isrslpqgsg nwaqlrpllv tfghdgrgha ltrrrrakrs pkhhsqrark knknorrhsl |
| 361 | yvdfsdvgwn dwivappgyq afychgdcpf pladhlnstn haivqtlvns vnssipkacc |
| 421 | vptelsaism lyldeydkvv lknyqemvve gcgcr |

38. Bmp5 (SEQ ID NO: 72)

| | |
|---|---|
| 1 | mhltvfllkg ivgflwscwv lvgyakgglg dnhvhssfiy rrlrnherre iqreilsilg |
| 61 | lphrprpfsp gkqassaplf mldlynamtn eenpeeseys vraslaeetr garkgypasp |
| 121 | ngyprriqls rttplttqsp plaslhdtnf lndadmvmsf vnlverdkdf shqrrhykef |
| 181 | rfdltqiphg eavtaaefri ykdrsnnrfe netikisiyq iikeytnrda dlflldtrka |
| 241 | qaldvgwlvf ditvtsnhwv inpqnnlglq lcaetgdgrs invksaglvg rqgpqskqpf |
| 301 | mvaffkasev llrsvraank rknqnrnkss shqdssrmss vgdyntseqk qackkhelyv |
| 361 | sfrdlgwqdw iiapegyaaf ycdgecsfpl nahmnatnha ivqtlvhlmf pdhvpkpcca |
| 421 | ptklnaisvl yfddssnvil kkyrnmvvrs cgch |

39. Bmp6 (SEQ ID NO: 73)

| | |
|---|---|
| 1 | mpglgrraqw lcwwwgllcs ccgppplrpp lpaaaaaaag gqllgdggsp grteqpppsp |
| 61 | qsssgflyrr lktqekremq keilsvlglp hrprplhglq qpqppalrqq eeqqqqqqlp |
| 121 | rgepppgrlk saplfmldly nalsadnded gasegerqqs wpheaasssq rrqpppgaah |
| 181 | plnrksllap gsgsggaspl tsaqdsafln dadmvmsfvn lveydkefsp rqrhhkefkf |
| 241 | nlsqipegev vtaaefriyk dcvmgsfknq tflisiyqvl qehqhrdsdl flldtrvvwa |
| 301 | seegwlefdi tatsnlwvvt pqhnmglqls vvtrdgvhvh praaglvgrd gpydkqpfmv |
| 361 | affkvsevhv rttrsassrr rqqsrnrstq sqdvarvssa sdynsselkt acrkhelyvs |
| 421 | fqdlgwqdwi iapkgyaany cdgecsfpln ahmnatnhai vqtlvhlmnp eyvpkpccap |
| 481 | tklnaisvly fddnsnvilk kyrnmvvrac gch |

40. Bmp7 (SEQ ID NO: 74)

| | |
|---|---|
| 1 | mhvrslraaa phsfvalwap lfllrsalad fsldnevhss fihrrlrsqe rremqreils |
| 61 | ilglphrprp hlqgkhnsap mfmldlynam aveegggpgg qgfsypykav fstqgpplas |
| 121 | lqdshfltda dmvmsfvnlv ehdkeffhpr yhhrefrfdl skipegeavt aaefriykdy |
| 181 | irerfdnetf risvyqvlqe hlgresdlfl ldsrtlwase egwlvfdita tsnhwvvnpr |
| 241 | hnlglqlsve tldgqsinpk lagligrhgp qnkqpfmvaf fkatevhfrs irstgskqrs |
| 301 | qnrsktpknq ealrmanvae nsssdqrqac kkhelyvsfr dlgwqdwiia pegyaayyce |
| 361 | gecafplnsy mnatnhaivq tlvhfinpet vpkpccaptq lnaisvlyfd dssnvilkky |
| 421 | rnmvvracgc h |

41. Bmp8a (SEQ ID NO: 75)

| | |
|---|---|
| 1 | mfqvvqeqsn resdlffldl qtlragdegw lvldvtaasd cwllkrhkdl glrlyveted |
| 61 | ghsvdpglag llgqraprsq qpfvvtffra spspirtpra vrplrrrqpk ktnelpqanr |
| 121 | lpgifddvhg shgrqvcrrh elyvsfqdlg wldwviapqg ysayycegec sfpldscmna |
| 181 | tnhailqslv hlmkpnavpk accaptklsa tsvlyydssn nvilrkhrnm vvkacgch |

42. Bambi (SEQ ID NO: 76)

| | |
|---|---|
| 1 | mdrhssyifi wlqlelcama vlltkgeirc ycdaahcvat gymckselsa cfsrlldpqn |
| 61 | snsplthgcl dslasttdic qakqarnhsg ttiptlecch edmcnyrglh dvlspprgea |
| 121 | sgqgnryqhd gsrnlitkvq eltsskelwf raaviavpia gglilvllim lalrmlrsen |
| 181 | krlqdqrqqm lsrlhysfhg hhskkgqvak ldlecmvpvs ghenccltcd kmrqadlsnd |
| 241 | kilslvhwgm ysghgklefv |

43. Bmper (SEQ ID NO: 77)

| | |
|---|---|
| 1 | mlwfsgvgal aerycrrspg itccvlllln csgvpmslas sfltgsvakc enegevlqip |
| 61 | fitdnpcimc vclnkevtrk rekcpvlsrd calaikqrga cceqckgcty egntynssfk |
| 121 | wqspaepcvl rqcqegvvte sgvrcvvhck nplehlgmcc ptcpgcvfeg vqyqegeefq |
| 181 | pegskctkcs ctggrtqcvr evcpilscpq hlshippgqc cpkclgqrkv fdlpfgsclf |
| 241 | rsdvydngss flydnctact crdstvvckr kcshpggcdq gqegcceecl 1rvppedikv |
| 301 | ckfgnkifqd gemwssinct icacvkgrte crnkqcipis scpqgkilnr kgccpictek |
| 361 | pgvctvfgdp hyntfdgrtf nfqgtcqyvl tkdcsspasp fqvlvkndar rtrsfswtks |
| 421 | velvlgesrv slqqhltvrw ngsrialpcr aphfhidldg yllkvttkag leiswdgdsf |
| 481 | vevmaaphlk gklcglcgny nghkrddlig gdgnfkfdvd dfaeswrves nefcnrpqrk |
| 541 | pvpelcqgtv kvklrahrec qklkswefqt chstvdyatf yrscvtdmce cpvhkncyce |
| 601 | sflaytracq regikvhwep qqncaatqck hgavydtcgp gciktcdnwn eigpcnkpcv |
| 661 | agchcpanlv lhkgrcikpv lcpqr |

44. COL3A1
COL3A1 protein [Homo sapiens]
ACCESSION AAH28178 (SEQ ID NO: 78)

| | |
|---|---|
| 1 | mmsfvqkgsw lllallhpti ilaqqeaveg gcshlgqsya drdvwkpepc qicvcdsgsv |
| 61 | lcddiicddq eldcpnpeip fgeccavcpq pptaptrppn gqgpqgpkgd pgppgipgrn |
| 121 | gdpgipgqpg spgspgppgi cescptgpqn yspqydsydv ksgvavggla gypgpagppg |
| 181 | ppgppgtsgh pgspgspgyq gppgepgqag psgppgppga igpsgpagkd gesgrpgrpg |
| 241 | erglpgppgi kgpagipgfp gmkghrgfdg rngekgetga pglkgenglp gengapgpmg |
| 301 | prgapgergr pglpgaagar gndgargsdg qpgppgppgt agfpgspgak gevgpagspg |
| 361 | sngapgqrge pgpqghagaq gppgppging spggkgemgp agipgapglm gargppgpag |
| 421 | angapglrgg agepgkngak gepgprgerg eagipgvpga kgedgkdgsp gepganglpg |
| 481 | aagergapgf rgpagpngip gekgpagerg apgpagprga agepgrdgvp ggpgmrgmpg |
| 541 | spggpgsdgk pgppgsqges grpgppgpsg prgqpgvmgf pgpkgndgap gkngerggpg |
| 601 | gpgpqgppgk ngetgpqgpp gptgpggdkg dtgppgpqgl qglpgtggpp gengkpgepg |
| 661 | pkgdagapga pggkgdagap gergppglag apglrggagp pgpeggkgaa gppgppgaag |
| 721 | tpglqgmpge rgglgspgpk gdkgepggpg adgvpgkdgp rgptgpigpp gpagqpgdkg |
| 781 | eggapglpgi agprgspger getgppgpag fpgapgqnge pggkgergap gekgeggppg |
| 841 | vagppgkdgt sghpgpigpp gprgnrgerg segspghpgq pgppgppgap gpccggvgaa |
| 901 | aiagiggeka ggfapyygde pmdfkintde imtslksvng qieslispdg srknparner |
| 961 | dlkfchpelk sgeywvdpnq gckldaikvf cnmetgetci sanplnvprk hwwtdssaek |
| 1021 | khvwfgesmd ggfqfsygnp elpedvldvq laflrllssr asqnityhck nsiaymdqas |
| 1081 | gnvkkalklm gsnegefkae gnskftytvl edgctkhtge wsktvfeyrt rkavrlpivd |
| 1141 | iapydiggpd qefgvdvgpv cfl |

collagen alpha-1 (III) chain preproprotein [Homo sapiens]
ACCESSION NP_000081 AAL13167 (SEQ ID NO: 79)

| | |
|---|---|
| 1 | mmsfvqkgsw lllallhpti ilaqqeaveg gcshlgqsya drdvwkpepc qicvcdsgsv |
| 61 | lcddiicddq eldcpnpeip fgeccavcpq pptaptrppn gqgpqgpkgd pgppgipgrn |
| 121 | gdpgipgqpg spgspgppgi cescptgpqn yspqydsydv ksgvavggla gypgpagppg |
| 181 | ppgppgtsgh pgspgspgyq gppgepgqag psgppgppga igpsgpagkd gesgrpgrpg |
| 241 | erglpgppgi kgpagipgfp gmkghrgfdg rngekgetga pglkgenglp gengapgpmg |
| 301 | prgapgergr pglpgaagar gndgargsdg qpgppgppgt agfpgspgak gevgpagspg |
| 361 | sngapgqrge pgpqghagaq gppgppging spggkgemgp agipgapglm gargppgpag |
| 421 | angapglrgg agepgkngak gepgprgerg eagipgvpga kgedgkdgsp gepganglpg |
| 481 | aagergapgf rgpagpngip gekgpagerg apgpagprga agepgrdgvp ggpgmrgmpg |
| 541 | spggpgsdgk pgppgsqges grpgppgpsg prgqpgvmgf pgpkgndgap gkngerggpg |
| 601 | gpgpqgppgk ngetgpqgpp gptgpggdkg dtgppgpqgl qglpgtggpp gengkpgepg |
| 661 | pkgdagapga pggkgdagap gergppglag apglrggagp pgpeggkgaa gppgppgaag |
| 721 | tpglqgmpge rgglgspgpk gdkgepggpg adgvpgkdgp rgptgpigpp gpagqpgdkg |
| 781 | eggapglpgi agprgspger getgppgpag fpgapgqnge pggkgergap gekgeggppg |
| 841 | vagppggsgp agppgpqgvk gergspggpg aagfpgargl pgppgsngnp gppgpsgspg |
| 901 | kdgppgpagn tgapgspgvs gpkgdagqpg ekgspgaqgp pgapgplgia gitgarglag |
| 961 | ppgmpgprgs pgpqgvkges gkpganglsg ergppgpqgl pglagtagep grdgnpgsdg |
| 1021 | lpgrdgspgg kgdrgengsp gapgapghpg ppgpvgpagk sgdrgesgpa gpagapgpag |
| 1081 | srgapgpqgp rgdkgetger gaagikghrg fpgnpgapgs pgpagqqgai gspgpagprg |
| 1141 | pvgpsgppgk dgtsghpgpi gppgprgnrg ergsegspgh pgqpgppgpp gapgpccggv |
| 1201 | gaaaiagigg ekaggfapyy gdepmdfkin tdeimtslks vngqieslis pdgsrknpar |
| 1261 | ncrdlkfchp elksgeywvd pnqgckldai kvfcnmetge tcisanplnv prkhwwtdss |
| 1321 | aekkhvwfge smdggfqfsy gnpelpedvl dvhlaflrll ssrasqnity hcknsiaymd |
| 1381 | qasgnvkkal klmgsnegef kaegnskfty tvledgctkh tgewsktvfe yrtrkavrlp |
| 1441 | ivdiapydig gpdqefgvdv gpvcfl |

In some embodiment, examples of useful positive markers for SSCs include BMPR1A, Axin2, BMP2, BMP3, BMP4, BMP6, BMP7, BMP8b, BMP15, Gremlin1, CD200, AlphaV, cathepsin K, Gli1, Ltf, Camp, Earl, Lcn2, Ngp, Chil3, Anxa1, Prg2, Elane, and Alox15.

All of the markers described herein and their respective homologs can be used as markers to identify, enrich, or isolate skeletal stem cells and suture stem cells of an animal subject, such as human or mouse.

### Identification, Isolation, and Enrichment of SSCs

In one aspect, the present application relates to the identification, isolation and enrichment of SSCs and in particular SuSCs. For example, these cells can be separated from a complex mixture of cells by techniques that identify or enrich for cells expressing one marker alone or in combination with other markers and characteristics as described herein. Mammalian SSCs, such as mouse SSCs and human SSCs may be characterized with the functional homologs of one or more the markers disclosed herein. Methods and compositions are provided for the separation and characterization of SSCs and bone progenitor cells. The cells may be separated from other cells by the expression of these specific cell markers such as surface markers.

Cells of interest, i.e. cells expressing a marker of choice, may be isolated or enriched for, that is, separated from the rest of the cell population, by a number of methods that are well known in the art. For example, flow cytometry, e.g. fluorescence activated cell sorting (FACS), may be used to separate the cell population based on the intrinsic fluorescence of the marker, or the binding of the marker to a specific fluorescent reagent, e.g. a fluorophore-conjugated antibody, as well as other parameters such as cell size and light scatter. In other words, the selection of the cells may be achieved by flow cytometry. Although the absolute level of staining may differ with a particular fluorochrome and reagent preparation, the data can be normalized to control. To normalize the distribution to control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype-matched control, but are not as intense as the most brightly staining cells normally found in the population. An alternative control may utilize a substrate having a defined density of marker on its surface, for example, a fabricated bead or cell line, which provides the positive control for intensity. Other methods of separation, i.e. methods by which selection of cells may be achieved, based upon markers include, for example, magnetic activated cell sorting (MACS), immunopanning, and laser capture microdissection.

Populations that are enriched by selecting for the expression of one or more markers will usually can have at least about 50%, e.g., 80% cells of the selected phenotype, more usually at least 90% cells and can be 95% of the cells, or more, of the selected phenotype.

In some examples, for the isolation of cells from a tissue, an appropriate solution may be used for dispersion or suspension. Such solution generally can be a balanced salt solution, e.g. normal saline, PBS, Hanks balanced salt solution, etc., supplemented with fetal calf serum or other naturally occurring factors, in conjunction with an acceptable buffer at low concentration, generally from 5-25 mM. Convenient buffers include HEPES, phosphate buffers, lactate buffers, etc. The tissue may be enzymatically and/or mechanically dissociated. In some embodiments, bone tissue is treated with a gentle protease, e.g. dispase, etc., for a period of time sufficient to dissociate the cells, then is gently mechanically dissociated.

An initial separation may select for cells by various methods known in the art, including elutriation, Ficoll-Hypaque or flow cytometry using the parameters of forward and obtuse scatter. Separation of the SSC population will then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, 7-AAD). Any technique may be employed which is not unduly detrimental to the viability of the selected cells.

The affinity reagents may be specific receptors or ligands for the cell surface molecules indicated above. Of particular interest is the use of antibodies as affinity reagents. The details of the preparation of antibodies and their suitability for use as specific binding members are well known to those skilled in the art. Depending on the specific population of cells to be selected, antibodies having specificity for a marker described herein can be contacted with the starting population of cells. Optionally, reagents specific for one or more of the other markers disclosed herein can also be included.

As is known in the art, the antibodies can be selected to have specificity for the relevant species, i.e. antibodies specific for human markers are used for the selection of human cells; antibodies specific for mouse markers are used in the selection of mouse cells, and the like.

These antibodies can be conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Examples of fluorochromes that can be used include phycobiliproteins, e.g. phycoerythrin and allophycocyanins, fluorescein and Texas red. Frequently each antibody is labeled with a different fluorochrome, to permit independent sorting for each marker.

The antibodies can be added to a suspension of cells and incubated for a period of time sufficient to bind the available cell surface antigens. The incubation usually can be at least about 5 minutes and usually less than about 2 hours. It is desirable to have a sufficient concentration of antibodies in the reaction mixture, such that the efficiency of the separation is not limited by the lack of antibodies. The appropriate concentration can be determined by titration. The medium in which the cells are separated can be any medium that maintains the viability of the cells. An exemplary medium can be phosphate buffered saline containing from about 0.1 to 20% BSA or FBS. Various media are commercially available and may be used according to the nature of the cells, including those described above, such as Dulbeccos Modified Eagle Medium (DMEM), Hank's Basic Salt Solution (HBSS), Dulbeccos phosphate buffered saline (dPBS), RPMI, Iscoves medium, PBS with 5 mM EDTA, etc., frequently supplemented with fetal calf serum, BSA, HSA, etc.

The labeled cells are then separated as to the phenotype described above. The separated cells may be collected in any appropriate medium that maintains the viability of the cells, usually having a cushion of serum at the bottom of the collection tube. Various media are commercially available and may be used according to the nature of the cells, including DMEM, HBSS, dPBS, RPMI, Iscoves medium, etc., frequently supplemented with fetal calf serum.

Compositions highly enriched for SSC can be achieved in this manner. The cell population can contain about 50% or more of the SSCs, and usually at or about 90% or more of SSCs, and can be as much as about 95% or more of SSCs. The enriched cell population may be used immediately, or be frozen at liquid nitrogen temperatures and stored for long periods of time, being thawed and capable of being reused. The cells can usually be stored in 10% DMSO, 50% FCS, 40% medium (e.g. RPMI 1640 medium). Once thawed, the cells may be expanded by the use of growth factors cells for proliferation and differentiation.

In some embodiments, the cells are identified, isolated, or enriched from a tissue. The tissue may be from any animal of interest. Examples include vertebrate animals, such as mammals and non-mammals, e.g., fishes, amphibians, and birds, which have similar skeletal structures and skeletal stem cells (Mork and Crump, Curr Top Dev Biol, 2015). Examples of mammals include humans, primates, domestic and farm animals, and zoo, laboratory or pet animals, such as dogs, cats, cattle, horses, sheep, pigs, goats, rabbits, rats, mice, etc. The cells may be established cell lines or they may be primary cells, where “primary cells”, “primary cell lines”, and “primary cultures” are used interchangeably herein to refer to cells and cells cultures that have been derived from a subject and allowed to grow in vitro for a limited number of passages.

The SSCs may be isolated from fresh or frozen cells, which may be from a neonate, a juvenile or an adult, and from tissues including skin, muscle, bone marrow, peripheral blood, umbilical cord blood, spleen, liver, pancreas, lung, intestine, stomach, adipose, and other tissues. The tissue may be obtained by biopsy or apheresis from a live donor or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such solution will generally be a balanced salt solution, e.g. normal saline, PBS, Hank's balanced salt solution, etc., supplemented with fetal calf serum or other naturally occurring factors, in conjunction with an acceptable buffer at low concentration, generally from 5-25 mM. Convenient buffers include HEPES, phosphate buffers, lactate buffers, etc.

### Cell Culturing, Expanding, and Differentiating

The SSCs described above may be cultured and maintained in vitro under various culture conditions. A suitable culture or nutrient medium may be liquid or semi-solid (e.g. containing agar, methylcellulose, etc.). The cell population may be suspended in an appropriate nutrient medium, such as DMEM, Iscove's modified DMEM or RPMI-1640, normally supplemented with fetal calf serum (about 1-25%), L-glutamine, a thiol, particularly 2-mercaptoethanol, and antibiotics. In one embodiment of the invention, the SSCs can be maintained in culture in the absence of feeder layer cells or the absence of serum, etc. Examples of a culture or nutrient medium include DMEM, DMEM/F12, DMEM high glucose, MEM, IMDM, Gibco StemPro™-34 SFM, Gibco Essential 8, Gibco Essential 6, Gibco MesenPRO, Gibco StemPro MSC, Gibco CTS KnockOut SR XenoFree, Corning NutriStem hPSC XF, and HyClone AdvanceSTEM.

The culture may contain antibiotics to prevent the growth of bacteria and fungi include. Examples of antibiotics include penicillin, streptomycin, Amphotericin B, Gentamicin, Puromycin, Hygromycin B, Ciprofloxacin, Chloramphenicol, Kanamycin, Neomycin, Blasticidin S, G418 Sulfate, Ampicillin, and Carbenicillin.

The culture or medium may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The growth factor may be any suitable growth factor. Examples include bone morphogenic protein (BMP); Indian hedgehog (IHH); transforming growth factor β (TGF β); bone morphogenetic proteins (BMPs) for example BMP-2, 4, 6 and 9; fibroblast growth factors (FGFs) like FGF-1 and 2; an epithelial cell growth factor (EGF); Wnt ligands and 0-catenin; insulin-growth factors (IGFs) like IGF-1 and IGF-2; Collagen-1; Runx2; Osteopontin; Osterix; vascular endothelial growth factor (VEGF); platelet derived growth factor (PDGF); osteoprotegerin (OPG); NEL-like protein 1 (NELL-1); or any combination thereof. For in detail disclosure of such growth factors, see, for example, Devescovi, V. et al. “Growth factors in bone repair” Chir Organi Mov 92, 161, 2008; James A. W. “Review of Signaling Pathways Governing MSC Osteogenic and Adipogenic Differentiation” Scientifica (Cairo) 2013; 2013: 684736; Carofino B. C. et al. “Gene therapy applications for fracture healing” J Bone Joint Surg Am, 90 (Suppl 1) (2008), pp. 99-110; Javed A. et al. “Genetic and transcriptional control of bone formation” Oral Maxillofac Surg Clin North Am. 2010; 22: 283-93; Chen G. et al. “TGF-β and BMP signaling in osteoblast differentiation and bone formation” Int. J. Biol. Sci. 2012; 8:272-288; Omitz, D. M. et al. “FGF signaling pathways in endochondral and intramembranous bone development and human genetic disease. Genes Dev. 16, 1446-1465 (2002); Krishnan V. et al. “Regulation of bone mass by Wnt signaling” J. Clin. Invest. 116 1202-1209 (2006); and Boyce B. F. et al. Arch Biochem Biophys. 473(2):139-146 (2008). The entire content of each of these publications is incorporated herein by reference.

The SSCs may be directed to differentiate along a specific path under conditions known in art. For example, SSCs can be directed to chondrogenesis by differentiation factors, which may be referred to herein as chondrogenesis factors. In some examples, the SSCs can be differentiated to a desired skeletal lineage cell. Specific embodiments include the skewing of differentiation from a skeletal stem cell to a chondrocyte by contacting with an effective dose of one or both of a VEGF inhibitor and a TGFβ inhibitor; and the use thereof in tissue repair. Other examples of skeletal cells that may be generated by the methods described herein pre-bone cartilage and stromal progenitor (pre-BCSP), BCSP, committed cartilage progenitor (CCP), bone progenitor, B-cell lymphocyte stromal progenitors (BLSP); 6C3 stroma, hepatic leukemia factor expressing stromal cell (HEC); and progeny thereof.

Cells contacted in vitro with the factors, e.g., the factors that promote reprogramming and/or promote the growth and/or differentiation of chondrocytes, and the like, may be incubated in the presence of the reagent(s) for about 30 minutes to about 24 hours, which may be repeated with a frequency of about every day to about every 4 days, e.g., every 2 days, every 3 days. The agent(s) may be provided to the cells one or more times, and the cells allowed to incubate with the agent(s) for some amount of time following each contacting event e.g. 16-24 hours, after which time the media is replaced with fresh media and the cells are cultured further.

After contacting the cells with the factors, the contacted cells may be cultured so as to promote the survival and differentiation of skeletal stem cells, chondrocytes, or progenitor cell populations defined herein. Methods and reagents for culturing cells are known in the art, any of which may be used here to grow and isolate the cells. For example, the cells (either pre- or post-contacting with the factors) may be plated on Matrigel or other substrates as known in the art. The cells may be cultured in media, supplemented with factors. Alternatively, the contacted cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by the use of growth factors and/or stromal cells associated with skeletal survival and differentiation.

Induced skeletal or chondrogenic cells produced by the above methods may be used in cell replacement or cell transplantation therapy to treat diseases. Specifically, the cells may be transferred to subjects suffering from a wide range of diseases or disorders with a skeletal or cartilaginous component.

In some cases, the cells or a sub-population of cells of interest may be purified or isolated or enriched from the rest of the cell culture prior to transferring to the subject. In some cases, one or more antibodies specific for a marker of cells of the skeletal/chondrogenic lineage or a marker of a sub-population of cells of the skeletal lineage are incubated with the cell population and those bound cells are isolated. In other cases, the cells or a sub-population of the cells express a marker that is a reporter gene, e.g. EGFP, dsRED, lacz, and the like, that is under the control of a specific promoter, which is then used to purify or isolate the cells or a subpopulation thereof.

### Spheres

In one aspect, the present application relates to a sphere of cells comprising one or more cells expressing one or more of the makers described herein. The sphere can be 20 to 200 μm in diameter, or have about 10 to 500 (e.g., 50-400, 100-300, etc.) cells per sphere. The sphere can comprise Axin2+ cells. To make such spheres, one can seed a population of suture stem cells or skeletal stem cells in a maintaining medium described above on a low attachment surface or ultra-low attachment surface and culture the cells or the progenies thereof for a period of time to form one or more spheres. The period of time can be 5-20 days, such as 5-15 days and 7-10 days.

A low attachment surface or ultra-low attachment surface refers to a surface for cell culture, which is treated to reduce or minimize cell adherence. Such a surface can have a neutral, hydrophilic hydrogel coating that greatly reduces the binding of attachment proteins. This minimizes cell attachment and spreading. A covalently bound hydrogel layer effectively inhibits cellular attachment and minimizes protein absorption, enzyme activation, and cellular activation. Cell culture with such ultra-low attachment surface is available from manufacturers such as CORNING.

The terms “sphere” and “sphere-like cell aggregate” are used interchangeably to refer to a cell aggregate having a stereoscopic shape close to globular. Examples of the stereoscopic shape close to globular include a globular shape which is a shape having a three-dimensional structure and indicating, when projected onto a two-dimensional surface, for example, a circle or an ellipse, and a shape formed by fusing a plurality of globular shapes (indicating, for example, when projected onto a two-dimensional surface, a shape formed by overlapping two to four circles or ellipses). The term “cell aggregate” refers to a ball-shaped cluster of cells or block of cells including pluripotent stem cells, such as SSCs. The cell aggregate may have a spherical shape. The cell aggregate may be a sphere. The sphere or aggregate is preferably formed by suspension culturing. The sphere or cell aggregate is a cluster of cells including undifferentiated pluripotent stem cells. The sphere or cell aggregate has the capability of producing various types of cells when the sphere/cell aggregate is cultured.

About 5% of mouse suture mesenchymal cells can be Bmpr1A+ cells and about 0.5% of suture mesenchymal cells may form a sphere. Most of the spheres include Bmpr1a+ cells. Therefore, approximately 10% of Bmpr1a+ cells form a sphere. For human stem cells, about 5% of suture mesenchymal cells are BMPR1A+ cells and about 0.1% of suture mesenchymal cells form a sphere in current culture condition. Most of the spheres include BMPR1A+ cells. Therefore, about 2% of BMPR1A+ suture mesenchymal cells form a sphere.

As disclosed herein, in primary sphere culture, there are 68% of spheres contain Axin2+ cells. In subsequent secondary and tertiary cultures, 100% of spheres contain Axin2+ cells. In theory, all spheres should be formed by Axin2+ cells as there are stem cells with “unlimited self-renewal” ability which require to form a sphere in sequential cultures for the true definition of the stem cell. An explanation for the primary culture is that some spheres (32%) are formed by progenitor cells with limited proliferation ability so can form sphere only in the primary culture but not secondary culture. Axin2+ stem cells are quiescent which gives rise to the sphere by asymmetric cell division. A sphere containing no Axin2+ cells means there is no Axin2+ stem cell with quiescent/slow-cycling features.

The cells, spheres, and methods described herein are useful in the development of an in vitro or in vivo model for bone function, for gene therapy, and for artificial organ construction. For example, the developing bones can serve as a source of growth factors and pharmaceuticals and for the production of viruses or vaccines, for in vitro toxicity and metabolism testing of drugs and industrial compounds, for gene therapy experimentation, for the construction of artificial transplantable bones, and for bone mutagenesis and carcinogenesis.

### Bone Regeneration Product

The cells described herein can be provided in combination with other types of cells, agents, materials, and structures for various uses, such as tissue engineering and treatment of any bone with a defect. Accordingly, this application provides a bone regeneration product or bone regeneration formulation comprising at least one skeletal stem cell and optionally at least one of such other types of cells, agents, material, and structure. To that end, the cells can be in three-dimensional (3D) synthetic, semi-synthetic, or living biological tissues.

In one example, the bone regeneration product/formulation comprises at least one SSC and at least one scaffold suitable for carrying the cell. The scaffold may comprise any 3D-printed scaffold suitable for carrying the cell. The bone regeneration product/formulation is suitable for dense bone regeneration, spongy bone regeneration, or a combination thereof. The bone regeneration product/formulation may further comprise a growth factor as mentioned above.

The scaffold can comprise various suitable materials, such as hydroxyapatite (HA) tricalcium phosphate (TCP), and a polymer. The polymer may be prepared by using photocurable polymers and/or monomers.

The scaffold may comprise a porous, 3D network of interconnected void spaces. The scaffold may be any scaffold suitable to incorporate the cells and/or growth factors disclosed herein to aid in forming a direct contact and/or an indirect contact of these cells and/or growth factors with a tissue (e.g. bone) for the regeneration of this tissue (e.g. bone). The scaffold may incorporate the cells and/or growth factors in any form, for example, by carrying, by supporting, by adsorbing, by absorbing, by encapsulating, by holding, and/or by adhering to the cells and/or growth factors.

The scaffold may have any shape or geometry. The scaffold may have any pore size. The scaffold may have any porosity (i.e. void volume.) The scaffold may have any form. The scaffold may have any mechanical strength.

Bone defects may form in different parts of an animal or a human body. These defects may have any shape and size. Scaffolds suitable for the treatment of such defects may have shapes, volumes and sizes that can, for example, fit to or resemble the defect shape and size. Such scaffolds may also have pore volumes, pore sizes, and/or pore shapes that resemble the bone for which the bone regeneration products that comprise such scaffolds are designed for their treatment. Such scaffolds may also have pores with pore sizes sufficiently small such that these scaffolds can contain the cells described herein within their porous structures and allow the bone regeneration product to be implanted and the treatment can be successfully carried out. The bone regeneration products/formulation can have a mechanical strength sufficient enough to handle load bearing conditions of their implantation to a body. It can also have a mechanical strength sufficient enough to handle load bearing conditions of bones during motion (e.g. walking) and/or weight of the bodies.

The scaffold may comprise any material. For example, the scaffold may comprise a non-resorbable material, resorbable material, or a mixture thereof. The resorbable material may be resorbed by the body of a patient and eventually replaced with healthy tissue. A “resorbable” material may comprise, for example, a biocompatible, bioabsorbable, biodegradable polymer, any similar material, or a mixture thereof.

A biocompatible material is a material that may be accepted by and to the function of a body of a patient without causing a significant foreign body response (such as, for example, an immune, inflammatory, thrombogenic, or like a response), and/or is a material that may not be clinically contraindicated for administration into a tissue or organ. The biodegradable material may comprise a material that is absorbable or degradable when administered in vivo and/or under in vitro conditions. Biodegradation may occur through the action of biological agents, either directly or indirectly.

The scaffold may comprise a solid, a liquid, or a mixture thereof. For example, the scaffold may be a paste. For example, the scaffold may comprise a paste comprising a mixture of hydroxyapatite and tricalcium phosphate (HA/TCP). This scaffold, for example, may be prepared by mixing hydroxyapatite (HA) and tricalcium phosphate (TCP) with a formulation comprising a liquid to prepare a paste. For example, the mesenchymal stem cell formulation may comprise a liquid; and mixing of such mesenchymal stem cell formulation with hydroxyapatite (HA) and tricalcium phosphate (TCP) may form a paste. This type of scaffold is called an HA/TCP scaffold herein.

A mixture comprising HA and TCP (HA/TCP) may be formed from equal amounts of HA and TCP in weight, for example, 50 wt % HA and 50 wt % TCP, unless otherwise stated. However, the mixture comprising HA and TCP may have any composition, for example, varying in the range of 0 wt % HA to 100 wt % TCP. For example, an HA concentration higher than 10 wt %, higher than 20 wt %, higher than 30 wt %, higher than 40 wt %, higher than 50 wt %, higher than 60 wt %, higher than 70 wt 00 higher than 80 wt %, or higher than 90 wt % is within the scope of this application.

The scaffolds described herein may comprise any biodegradable polymer. For example, the scaffold may comprise a synthetic polymer, naturally occurring polymer, or a mixture thereof. Examples of suitable biodegradable polymers may be polylactide (PLA), polyglycolide (PGA), poly(lactide-co-glycolide) (PLGA), poly-E-caprolactone, polydioxanone trimethylene carbonate, polyhybroxyalkonates (e.g., poly(hydroxybutyrate)), poly(ethyl glutamate), poly(DTH iminocarbony (bisphenol A iminocarbonate), poly(ortho ester), polycyanoacrylates, fibrin, casein, serum albumin, collagen, gelatin, lecithin, chitosan, alginate, poly-amino acids (such as polylysine), and a mixture thereof.

The scaffold may further comprise one or more of the growth factors described above. The growth factor may be continuously released to the surrounding (bone) tissue after the bone regeneration product is implanted into the bone defect site. The release rate of the growth factor may be controlled through the scaffolds' chemical composition and/or pore structure.

The scaffold may be manufactured by any technique. For example, the scaffold may be manufactured by hand, and/or by using a machine. For example, the scaffold may be manufactured by additive manufacturing and/or manufacturing. For example, the scaffold may be manufactured using a combination of more than one such manufacturing technique.

In one embodiment, the cells are used in a “bio-printing” process to generate a spatially-controlled cell pattern using a 3D printing technology. Any bio-printing or bio-fabricating process known in the art can be used, e.g., as described in U.S. Pat. App. Pub. Nos. 20140099709, 20140093932, 20140274802, 20140012407, 20130345794, 20130190210 and 20130164339; and U.S. Pat. No. 8,691,974.

For example, in one embodiment, a printer cartridge is filled with a suspension of SSCs or SSC spheres and a gel. The alternating patterns of the gel and cells or spheres are printed using a standard print nozzle. In an alternative embodiment, a NOVOGEN (San Diego, Calif.) MMX™, or Organovo Holdings, Inc., bioprinters can be used for 3D bioprinting. These and equivalent “bio-printers” can be optimized to “print”, or fabricate, bone tissue, cartilage tissue, and other tissues, all of which are suitable for surgical therapy and transplantation.

Any 3D printing technique may be used to manufacture the scaffold. The 3D printing technique or additive manufacturing (AM) may be a process for making a physical object from a 3D digital model, typically by laying down many successive thin layers of material. Such thin layers of material may be formed under computer control. Examples of the 3D printing technologies may be Stereolithography (SLA), Digital Light Processing (DLP), Fused deposition modeling (FDM), Selective Laser Sintering (SLS), Selective laser melting (SLM), Electronic Beam Melting (EBM), Laminated object manufacturing (LOM), Binder jetting (BJ), Material Jetting (MJ) or Wax Casting (WC), or a combination thereof.

The scaffold, including the 3D printed scaffold, for example, may be manufactured by using a formulation comprising polycaprolactone dimethacrylate (PCLDA), calcium phosphate, HA, TCP, polyethylene glycol diacrylate (PEGDA), gelatin methacryloyl (GelMA), or a mixture thereof.

This bone regeneration product/formulation can be used in regenerating bone. Such as bone regeneration method may comprise implanting the bone regeneration product/formulation in or across a bone defect. This bone defect may be any bone defect, such as a bone defect of a long bone. The size of this bone defect may be any size. For example, the size of this bone defect may be a critical size. The bone defect may be formed due to a congenital bone malformation. The congenital bone defect may be a defect related to a cleft lip and/or a cleft palate. The bone defect may be formed because of surgery, accident, and/or disease. This bone defect may be formed as a result of surgery carried out to treat craniosynostosis.

### Pharmaceutical Composition

The present application further provides a composition comprising (i) a carrier and (ii) one or more cells obtained according to methods described herein or cells (such as progeny cells) derived therefrom. The composition can be a pharmaceutical composition where the carrier is a pharmaceutically acceptable carrier.

A composition for pharmaceutical use, e.g., a scaffold or implant with cells and/or factors, can include, depending on the formulation desired, pharmaceutically acceptable, non-toxic carriers of diluents, which are defined as vehicles commonly used to formulate pharmaceutical compositions for animal or human administration. The diluent can be selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide (e.g., a growth factor), the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see Langer, Science 249:1527-1533 (1990).

The pharmaceutical composition described herein, e.g., SSCs alone or in combinations with various factors, can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

Data obtained from cell culture and/or animal studies can be used in formulating a range of dosages for humans. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxin, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression of the disease condition as required. Utilizing animal data, and other information available for the agent, a clinician can determine the maximum safe dose for an individual, depending on the route of administration. For instance, an intravenously administered dose may be more than a locally administered dose, given the greater body of fluid into which the therapeutic composition is being administered. Similarly, compositions that are rapidly cleared from the body may be administered at higher doses, or in repeated doses, in order to maintain a therapeutic concentration. Utilizing ordinary skills, the competent clinician will be able to optimize the dosage of a particular therapeutic in the course of routine clinical trials.

Mammalian species that may be treated with the present methods include canines and felines; equines; bovines; ovines; etc. and primates, particularly humans. Animal models, particularly small mammals, e.g. murine, lagomorpha, etc. may be used for experimental investigations.

### Uses

The cells, compositions, formulations, and products disclosed herein can be used for various purposes including treating related disorders and in tissue engineering.

In some embodiments, the cells, compositions, formulations, and products disclosed herein can be used in the treatment of a subject, such as a human patient, in need of bone or cartilage replacement therapy. Examples of such subjects can be subjects suffering from conditions associated with the loss of cartilage or bone from osteoporosis, osteoarthritis, genetic defects, disease, etc. Patients having diseases and disorders characterized by such conditions will benefit greatly from a treatment protocol of the pending claimed invention.

An effective amount of the pharmaceutical composition is the amount that will result in an increase in the number of chondrocytes, skeletal cells, cartilage or bone mass at the site of implant, and/or will result in a measurable reduction in the rate of disease progression in vivo. For example, an effective amount of a pharmaceutical composition will increase bone or cartilage mass by at least about 5%, at least about 10%, at least about 20%, preferably from about 20% to about 50%, and even more preferably, by greater than 50% (e.g., from about 50% to about 100%) as compared to the appropriate control, the control typically being a subject not treated with the composition.

The methods described above can be used in combined therapies with, e.g. therapies that are already known in the art to provide relief from symptoms associated with the aforementioned diseases, disorders, and conditions. The combined use of a pharmaceutical composition described herein and these other agents may have the advantages that the required dosages for the individual drugs are lower, and the effects of the different drugs are complementary.

In some embodiments, an effective dose of SSCs described herein, preferably SuSCs, are provided in an implant or scaffold for the regeneration of skeletal or cartilaginous tissue. An effective cell dose may depend on the purity of the population. In some embodiments, an effective dose delivers a dose of cells of at least about 10², about 103, about 104, about 105, about 10⁶, about 107, about 108, about 10⁹, or more cells, which stem cells may be present in the cell population at a concentration of about 1%, about 5%, about 10%, about 20%, about 30%, about 40%, about 50%, about 60%, about 70%, about 80%, about 90% or more.

The present application provides methods and compositions for the differentiation of SSCs, including SuSCs, into cells such as chondrocytes. The cells produced by the methods are useful in providing a source of fully differentiated and functional cells for research, transplantation, and the development of tissue engineering products for the treatment of human disease and traumatic injury repair.

### Tissue Engineering

Tissue engineering is the use of a combination of cells, engineering and materials methods, and suitable biochemical and physio-chemical factors to improve or replace biological functions. For example, cells may be implanted or seeded into an artificial structure capable of supporting three-dimensional tissue formation. These structures, referred to herein as a matrix or scaffold, allow cell attachment and migration, deliver and retain cells and biochemical factors, and enable diffusion of vital cell nutrients and expressed products. High porosity and adequate pore size are important to facilitate cell seeding and diffusion throughout the whole structure of both cells and nutrients. Biodegradability is often a factor since scaffolds may be absorbed by the surrounding tissues without the necessity of surgical removal. The rate at which degradation occurs has to coincide as much as possible with the rate of tissue formation: this means that while cells are fabricating their own natural matrix structure around themselves, the scaffold is able to provide structural integrity within the body and eventually it will break down leaving the neotissue, newly formed tissue which will take over the mechanical load. Injectability is also important for clinical uses.

Many different materials (natural and synthetic, biodegradable and permanent) have been investigated and can be used for tissue engineering matrices or scaffolds. Examples include Puramatrix, polylactic acid (PLA), polyglycolic acid (PGA) and polycaprolactone (PCL), and combinations thereof. Scaffolds may also be constructed from natural materials, e.g. proteins such as collagen, fibrin, etc; polysaccharidic materials, such aschitosan; alginate, glycosaminoglycans (GAGs) such as hyaluronic acid, etc. Functionalized groups of scaffolds may be useful in the delivery of small molecules (drugs) to specific tissues. Another form of scaffold under investigation is decellularised tissue extracts whereby the remaining cellular remnants/extracellular matrices act as the scaffold.

### Treatment Methods

The above-described cells, compositions, formulations, products, and methods can be used to treat various bone defects such as large craniofacial bone defects, craniosynostosis, and bone fractures. Examples include (A) Bone fracture caused by various conditions, e.g. osteoporosis and osteopenia, (B) Large bone defects caused by various conditions, including cancer surgeries, congenital malformation (e.g., Cleidocranial Dysplasia, Cleft Palate, Facial Cleft, Treacher-Collins, fibrous dysplasia), trauma, and progressive deforming diseases, and (C) the stem cell-based therapy may be used to substitute any procedure involving bone graft.

Large craniofacial bone defects, which are caused by various conditions, including trauma, infection, tumors, congenital disorders, and progressive deforming diseases, are major health issues. The autologous bone graft is a recommended procedure for extensive skeletal repairs but their success remains highly challenging owing to several limitations. Consequently, alternative approaches have been explored. Stem cell-based therapy is particularly attractive and promising, in light of the characterization of skeletal stem cells in craniofacial and body skeletons. Craniofacial bone is mainly formed through intramembranous ossification, a process different from the endochondral ossification required for the body skeleton. Because of the distinct properties of the stem cells of the craniofacial and body skeletons, it is necessary to study each type of skeletal stem cells. Suture stem cells (SuSCs) are the stem cell population that is naturally programmed to form intramembranous bones during craniofacial skeletogenesis. The lack of a cell marker and in particulate a cell surface marker for stem cell isolation and the inability to maintain stemness characteristics ex vivo are two critical hurdles that restrict further advances in the field of skeletal regeneration. The cells, compositions, and methods disclosed herein address this unmet need.

Craniosynostosis, which affects one in ˜2,500 individuals, is one of the most common congenital deformities and is caused by premature suture closure. The suture serving as the growth center for calvarial morphogenesis is the equivalent of the growth plate in the long bone. Excessive intramembranous ossification caused by genetic mutation promotes suture fusion. An example is the genetic loss of function of AXIN2, which causes craniosynostosis in mice and humans. In 2010, it was found that craniosynostosis can also be caused by mesenchymal cell fate switching, leading to suture closure through endochondral ossification. By regulating the interplay between bone morphogenetic protein (BMP) and fibroblast growth factor (FGF) pathways, Axin2-mediated Wnt signaling determines skeletogenic commitment into an osteogenic or chondrogenic lineage. The multipotency further supports the existence of skeletal stem cells within the suture mesenchyme. Because Axin2 expression in the presumptive niche site was tightly linked to suture patency, Axin2-expressing SuSCs was identified as essential for calvarial development, homeostasis, and injury-induced repair. The Axin2-positive (Axin2+) SuSCs qualified for the modem, rigorous stem cell definition: They exhibit not only long-term self-renewal, clonal expansion, differentiation, and multipotency but also the ability to repair skeletal defects by direct engraftment and replacement of damaged tissue.

When used for tissue engineering or treatment methods, the SSCs and/or combinations of factors for lineage differentiation can be provided for in vivo use in a cellular solution, in which hydrating solutions, suspensions, or other fluids that contain the cells or factors that are capable of differentiating into bone or cartilage.

Bone or cartilage graft devices and compositions may be provided that are optimized in terms of one or more compositions, bioactivity, porosity, pore size, protein binding potential, degradability, or strength for use in both load bearing and non-load bearing cartilage or bone grafting applications. Preferably, graft materials are formulated so that they promote one or more processes involved in bone or cartilage healing which can occur with the application of a single graft material: chondrogenesis, osteogenesis, osteoinduction, and osteoconduction. Chondrogenesis is the formation of new cartilaginous structures. Osteogenesis is the formation of new bone by the cells contained within the graft. Osteoinduction is a chemical process in which molecules contained within the graft (for example, bone morphogenetic proteins, and TGF-β) convert the patient or other bone progenitor cells into cells that are capable of forming bone. Osteoconduction is a physical effect by which the matrix of the graft forms a scaffold on which bone forming cells in the recipient are able to form new bone.

Inclusion of the factors and/or cells described herein can be used to facilitate the replacement and filling of cartilage or bone material in and around pre-existing structures. In some embodiments, the cells produce chondrocytes first, followed by the deposition of extra cellular matrix and bone formation. The bone grafts can provide an osteoconductive scaffold comprising calcium phosphate ceramics which provide a framework for the implanted progenitor cells and local osteocytes to differentiate into bone forming cells and deposit new bone. The use of calcium phosphate ceramics can provide for a slow degradation of the ceramic, which results in a local source of calcium and phosphate for bone formation. Therefore, new bone can be formed without calcium and phosphate loss from the host bone surrounding the defect site. Calcium phosphate ceramics are chemically compatible with that of the mineral component of bone tissues. Examples of such calcium phosphate ceramics include calcium phosphate compounds and salts, and combinations thereof.

In some embodiments, the cells and/or factors can be prepared as an injectable paste. A cellular suspension can be added to one or more cells to form an injectable hydrated paste. The paste can be injected into the implant site. In some embodiments, the paste can be prepared prior to implantation and/or store the paste in the syringe at sub-ambient temperatures until needed. In some embodiments, the application of the composite by injection can resemble a bone cement that can be used to join and hold bone fragments in place or to improve adhesion of, for example, a hip prosthesis, for replacement of damaged cartilage in joints, and the like. Implantation in a non-open surgical setting can also be performed.

In other embodiments, the cells and/or factors can be prepared as formable putty. A cellular suspension can be added to one or more powdered minerals to form a putty-like hydrated graft composite. The hydrated graft putty can be prepared and molded to approximate any implant shape. The putty can then be pressed into place to fill a void in the cartilage, bone, tooth socket, or another site. In some embodiments, graft putty can be used to repair defects in non-union bone or in other situations where the fracture, hole or void to be filled is large and requires a degree of mechanical integrity in the implant material to both fill the gap and retain its shape.

The methods described herein can be used for treating a cartilage or bone lesion, or injury, in a human or other animal subjects, comprising applying to the site a composition comprising cells and/or factors described herein, which may be provided in combinations with cements, factors, gels, etc. As referred to herein such lesions include any condition involving skeletal, including cartilaginous, tissue that is inadequate for physiological or cosmetic purposes. Such defects include those that are congenital, the result of disease or trauma, and consequent to surgical or other medical procedures. Such defects include, for example, a bone defect resulting from injury, defect brought about during the course of surgery, osteoarthritis, osteoporosis, infection, malignancy, developmental malformation, and bone breakages such as simple, compound, transverse, pathological, avulsion, greenstick and comminuted fractures. In some embodiments, a bone defect is a void in the bone that requires filling with a bone progenitor composition.

The cells described herein can also be genetically altered in order to enhance their ability to be involved in tissue regeneration or to deliver a therapeutic gene to a site of administration. To that end, a vector can be designed using the known encoding sequence for the desired gene, operatively linked to a promoter that is either pan-specific or specifically active in the differentiated cell type. Of particular interest are cells that are genetically altered to express a bone morphogenic protein, such as BMP-2 or BMP-4. See WO 99/39724. Production of these or other growth factors at the site of administration may enhance the beneficial effect of the administered cell, or increase the proliferation or activity of host cells neighboring the treatment site.

### Research Uses and Drug Screening

The cells described herein can also be used as a research or drug discovery tool, for example, to evaluate the phenotype of a genetic disease, e.g. to better understand the etiology of the disease, to identify target proteins for therapeutic treatment, to identify candidate agents with disease-modifying activity, e.g. to identify an agent that will be efficacious in treating the subject. For example, a candidate agent may be added to a cell culture comprising the SSC derived from a subject, and the effect of the candidate agent assessed by monitoring output parameters such as survival, the ability to form bone or cartilage, and the like, by methods described herein and in the art.

Parameters are quantifiable components of cells, particularly components that can be accurately measured, desirably in a high throughput system. A parameter can be any cell component or cell product including cell surface determinant, receptor, protein or conformational or posttranslational modification thereof, a lipid, a carbohydrate, an organic or inorganic molecule, a nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include the mean, median value or variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Examples of candidate agents include organic molecules comprising functional groups necessary for structural interactions, particularly hydrogen bonding, and typically include at least an amine, carbonyl, hydroxyl, or carboxyl group, frequently at least two of the functional chemical groups. The candidate agents can comprise cyclical carbon or heterocyclic structures and/or aromatic or polyaromatic structures substituted with one or more of the above functional groups. Candidate agents can be biomolecules, including peptides, polynucleotides, saccharides, fatty acids, steroids, purines, pyrimidines, derivatives, structural analogs, or combinations thereof. Further examples include pharmacologically active drugs, genetically active molecules, etc. Compounds of interest include chemotherapeutic agents, hormones or hormone antagonists, etc. Exemplary of the pharmaceutical agents suitable for this invention are those described in, “The Pharmacological Basis of Therapeutics,” Goodman and Gilman, McGraw-Hill, New York, N.Y., (1996), Ninth edition.

In an example, the cells and methods described herein are useful for screening candidate agents for activity in modulating cell conversion into cells of a skeletal or chondrogenic lineage, e.g. chondrocytes, osteoblasts, or progenitor cells thereof. In screening assays for biologically active agents, the cells can be contacted with a candidate agent of interest in the presence of the cell reprogramming or differentiation system or an incomplete cell reprogramming or differentiation system, and the effect of the candidate agent is assessed by monitoring output parameters such as the level of expression of genes specific for the desired cell type, as is known in the art, or the ability of the cells that are induced to function like the desired cell type; etc. as is known in the art.

### Kits

Provided here are kits for identifying or isolating or enriching a suture stem cell or a skeletal stem cell.

The kit may comprise one or more agents (e.g., antibodies or nucleic acid probes) that are specific to one or more makers described herein. In one example, the kit comprises a system for contacting a biological sample comprising one or more marker-specific antibodies or antigen-binding fragments thereof and instructions for use thereof. In one embodiment, the kit further comprises a culturing system (e.g., a cell culture medium or a cell culture device or both) for culturing, maintaining, or expanding the population of cells, and instructions for use thereof. The kit may comprise one or more agents for measuring the marker protein or mRNA transcript level.

The agents may be, for example, reagents for carrying out analysis methods for detecting a protein or measuring protein levels. Examples of such analysis methods include, but are not limited to, FACS, immunohistostaining assay, Western blotting, ELISA, radioimmunoassay, radioimmunodiffusion, Ouchterlony immunodiffusion assay, rocket immunoelectrophoresis assay, immunoprecipitation assay, complement fixation assay, protein chip assay, etc. This kit may comprise reagents that detect antibodies forming antigen-antibody complexes, for example, labeled secondary antibodies, chromophores, enzymes (e.g., conjugated with antibodies) and their substrates. In addition, it may comprise an antibody specific to a control protein for quantification. The amount of antigen-antibody complexes formed may be quantitatively determined by measuring the signal intensity of a detection label. Such a detection label may be selected from the group consisting of, but not necessarily limited to, enzymes, fluorescent substances, ligands, luminescent substances, microparticles, redox molecules and radioactive isotopes.

For detecting mRNA transcripts, a kit may contain reagents for extracting mRNA, measuring the expression level of mRNA of a marker described herein (e.g., probes or PCR primers). The mRNA expression can be measured by anyone selected from the group consisting of, but not limited to, in situ hybridization, polymerase chain reaction (PCR), reverse transcription-polymerase chain reaction (RT-PCR), real-time PCR, RNase protection assay (RPA), microarray, and Northern blotting.

Also provided is a kit comprising SSCs, spheres, a composition, a product, or a formulation described herein. Optionally, the kit contains one or more of the scaffold described herein. The SSC, spheres, composition, product, or formulation be presented in a kit, pack or dispenser, which may contain one or more unit dosage forms containing the active ingredient. The kit, for example, may comprise metal or plastic foil, such as a blister pack. The kit, pack, or dispenser may be accompanied by instructions for administration.

### Definitions

The terms “polypeptide” or “protein” are used interchangeably herein to refer to a polymer of amino acid residues and their derivatives. The terms also apply to amino acid polymers in which one or more amino acid residues are an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated.

Polypeptides and proteins can be produced by a naturally occurring and non-recombinant cell; or it is produced by a genetically engineered or recombinant cell, and comprise molecules having the amino acid sequence of the native protein, or molecules having deletions from, additions to, and/or substitutions of one or more amino acids of the native sequence. The terms “polypeptide” and “protein” specifically encompass antigen binding proteins, antibodies, or sequences that have deletions from, additions to, and/or substitutions of one or more amino acids of an antigen-binding protein. The term “polypeptide fragment” refers to a polypeptide that has an amino-terminal deletion, a carboxyl-terminal deletion, and/or an internal deletion as compared with the full-length protein. Such fragments may also contain modified amino acids as compared with the full-length protein. In certain embodiments, fragments are about five to 500 amino acids long. For example, fragments may be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains.

The term “isolated polypeptide” refers to a polypeptide that has been separated from at least about 50 percent of polypeptides, peptides, lipids, carbohydrates, polynucleotides, or other materials with which the polypeptide is naturally found when isolated from a source cell. Preferably, the isolated polypeptide is substantially free from any other contaminating polypeptides or other contaminants that are found in its natural environment that would interfere with its therapeutic, diagnostic, prophylactic or research use.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is composed of a heavy chain variable region (abbreviated herein as V_{H}) and a heavy chain constant region. The heavy chain constant region is composed of three domains, C_{H}1, C_{H}2 and C_{H}3. Each light chain is composed of a light chain variable region (abbreviated herein as V_{L}) and a light chain constant region. The light chain constant region is composed of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

Accordingly, the terms “antibody” and “antibodies” include full-length antibodies, antigen-binding fragments of full-length antibodies, and molecules comprising antibody CDRs, VH regions or VL regions. Examples of antibodies include monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), scFv-Fcs, camelid antibodies (e.g., llama antibodies), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies disclosed herein refer to polyclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies disclosed herein are IgG antibodies, or a class (e.g., human IgG₁ or IgG₄) or subclass thereof. In a specific embodiment, the antibody is a humanized monoclonal antibody.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody.

Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993)); (iv) a Fd fragment consisting of the V_{H} and C_{H}I domains; (v) a Fv fragment consisting of the V_{L} and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds to a specific antigen is substantially free of antibodies that specifically bind antigens other than the specific antigen). An isolated antibody can be substantially free of other cellular material and/or chemicals.

As used herein, the term “administering” refers to the delivery of compositions of the present invention by any suitable route. Cells can be administered in a number of ways including, but not limited to, parenteral (such a term referring to intravenous and intra-arterial as well as other appropriate parenteral routes), intrathecal, intraventricular, intraparenchymal, intracisternal, intracranial, intrastriatal, intranigral, intranasal, intraperitoneal, intramuscular, subcutaneous, intradermal, transdermal, or transmucosal administration, among others which term allows cells to migrate to the ultimate target site where needed. Multiple units of cells can be administered simultaneously or consecutively (e.g., over the course of several minutes, hours, or days) to a patient.

The terms “grafting” and “transplanting” and “graft” and “transplantation” are used to describe the process by which cells are delivered to the site where the cells are intended to exhibit a favorable effect, such as repairing damage to a patient's bone or cartilage. Cells can also be delivered in a remote area of the body by any mode of administration as described above, relying on cellular migration to the appropriate area to effect transplantation.

The term “therapeutic composition” or pharmaceutical composition” refers to the combination of an active agent with a carrier, inert or active, making the composition especially suitable for diagnostic or therapeutic use in vivo or ex vivo.

As used herein, “therapeutic cells” refers to a cell population that ameliorates a condition, disease, and/or injury in a patient. Therapeutic cells may be autologous (i.e., derived from the patient), allogeneic (i.e., derived from an individual of the same species that is different from the patient), or xenogeneic (i.e., derived from a different species than the patient). Therapeutic cells may be homogenous (i.e., consisting of a single cell type) or heterogeneous (i.e., consisting of multiple cell types). The term “therapeutic cell” includes both therapeutically active cells as well as progenitor cells capable of differentiating into a therapeutically active cell.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for the delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The term “subject” includes human and non-human animals. The preferred subject for treatment is a human. As used herein, the terms “subject” and “patient” are used interchangeably irrespective of whether the subject has or is currently undergoing any form of treatment. In one embodiment, the subject is a human. In another embodiment, the subject is an experimental, non-human animal or animal suitable as a disease model.

The term “patient” is used herein to describe an animal, preferably a human, to whom treatment, including prophylactic treatment, with the cells according to the present invention, is provided. The term “donor” is used to describe an individual (animal, including a human) who or which donates cells or tissue for use in a patient.

The term “primary culture” denotes a mixed cell population of cells from an organ or tissue within an organ. The word “primary” takes its usual meaning in the art of tissue culture.

A “tissue” refers to a group or layer of similarly specialized cells which together perform certain special functions.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or maybe therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. The terms “prevent,” “preventing,” “prevention,” “prophylactic treatment” and the like refer to reducing the probability of developing a disorder or condition in a subject, who does not have, but is at risk of or susceptible to developing a disorder or condition. “Ameliorating” generally refers to the reduction in the number or severity of signs or symptoms of a disease or disorder.

A “prophylactic treatment” includes a treatment administered to a subject who does not display signs or symptoms of a condition such that treatment is administered for the purpose of diminishing, preventing, or decreasing the risk of developing the condition. A “therapeutic treatment” includes a treatment administered to a subject who displays symptoms or signs of a condition and is administered to the subject for the purpose of reducing the severity or progression of the condition. A therapeutic treatment can also partially or completely resolve the condition.

An “effective amount” generally means an amount that provides the desired local or systemic effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and the amount of time since the injury occurred or the disease began. One skilled in the art will be able to determine the effective amount for a given subject based on these considerations which are routine in the art. As used herein, “effective dose” means the same as “effective amount.”

As used herein, the term “stem cells” refers to cells with the ability to both replace themselves and to differentiate into more specialized cells. Their self-renewal capacity generally endures for the lifespan of the organism. A pluripotent stem cell can give rise to all the various cell types of the body. A multipotent stem cell can give rise to a limited subset of cell types. For example, a hematopoietic stem cell can give rise to the various types of cells found in blood, but not to other types of cells. Multipotent stem cells can also be referred to as somatic stem cells, tissue stem cells, lineage-specific stem cells, and adult stem cells. The non-stem cell progeny of multipotent stem cells are progenitor cells (also referred to as restricted-progenitor cells). Progenitor cells give rise to fully differentiated cells, but a more restricted set of cell types than stem cells. Progenitor cells also have comparatively limited self-renewal capacity; as they divide and differentiate they are eventually exhausted and replaced by new progenitor cells derived from their upstream multipotent stem cell.

The term “skeletal stem cell” refers to a multipotent and self-renewing cell capable of generating bone marrow stromal cells, skeletal cells, and chondrogenic cells. By self-renewing, it is meant that when they undergo mitosis, they produce at least one daughter cell that is a skeletal stem cell. By multipotent it is meant that it is capable of giving rise to progenitor cells (skeletal progenitors) that give rise to all cell types of the skeletal system. They are not pluripotent, that is, they are not capable of giving rise to cells of other organs in vivo.

Skeletal stem cells can be reprogrammed from non-skeletal cells, including without limitation mesenchymal stem cells, and adipose tissue containing such cells. Reprogrammed cells may be referred to as induced skeletal stem cells, or iSSC. “iSSC” arise from a non-skeletal cell by experimental manipulation. Induced skeletal cells have characteristics of functional SSCs derived from nature, that is, they can give rise to the same lineages. Human SSC cell populations can be negative for expression of certain markers such as CD45, CD235, Tie2, and CD31; and positively express others, such as podoplanin (PDPN). The mouse skeletal lineage can be characterized as CD45−, Ter119−, Tie2−, αv integrin+. The mouse SSC can be further characterized as Thy1-6C3− CD105− CD200+.

Suture stem cells (SuSCs) refer to a population of skeletal stem cells from the suture mesenchyme that exhibit long-term self-renewal, clonal expansion, and multipotency. These SuSCs reside in the suture midline and serve as the skeletal stem cell population responsible for calvarial development, homeostasis, injury repair, and regeneration. Suture stem cells are the stem cell population that is naturally programmed to form intramembranous bones during craniofacial skeletogenesis.

Chondrocytes (cartilage cells)” refers to cells that are capable of expressing characteristic biochemical markers of chondrocytes, including but not limited to collagen type II, chondroitin sulfate, keratin sulfate and characteristic morphologic markers of a chondrocyte, including but not limited to the rounded morphology observed in culture, and able to secrete collagen type II, including but not limited to the generation of tissue or matrices with hemodynamic properties of cartilage in vitro.

As used herein, the phrase “maintaining stem cells” refers not just to culturing the stem cells in a manner preserving their viability, but also to retaining their functionality as stem cells, that is, to being self-renewing and capable of giving rise to the full range of progenitor lineages appropriate to the particular type of stem cell (these two functions together “regenerative activity”). One way of demonstrating that stem cells have been successfully maintained is through an engraftment experiment in which all the appropriate cell types (bearing a genetic marker distinguishing them from the host) are observed to arise from the graft and remain present over an extended period, for example, 4 months.

As used herein, the phrase “expanding stem cells” refers not just to maintaining the stem cells but to culturing the stem cells in a manner that the number of stem cells in the culture increases. One way of demonstrating that stem cells have been successfully expanded is an engraftment experiment comparing the percentage of donor-derived cells obtained from transplants of cultured and freshly isolated stem cells. The comparison is based on transplanting the same number of freshly isolated stem cells as were originally placed in culture. An increased percentage of donor-derived cells in the recipients of the cultured stem cells as compared to in the recipients of the freshly-isolated stem cells is consistent with the successful expansion of the stem cells in culture.

A “marker” or “biomarker” is a molecule useful as an indicator of a biologic state in a subject. The marker or biomarkers disclosed herein can be polypeptides that exhibit a change in expression or state, which can be correlated with the development, differentiation, or fate of a cell. In addition, the markers disclosed herein are inclusive of messenger RNAs (mRNAs) encoding the marker polypeptides, as the measurement of a change in the expression of an mRNA can be correlated with changes in the expression of the polypeptide encoded by the mRNA. As such, determining an amount of a biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

In the context of skeletal stem cells, a “marker” or “biomarker” means that, in cultures or tissues comprising cells that have been programmed to become skeletal stem cells, the marker is expressed only by the cells of the culture or tissue that will develop, are developing, and/or have developed into skeletal stem cells. It will be understood by those of skill in the art that the stated expression levels reflect detectable amounts of the marker protein or mRNA on or in the cell. A cell that is negative for staining (the level of binding of a marker-specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still, be characterized as “positive.”

Standard gene/protein nomenclature guidelines generally stipulate gene name abbreviations/symbols for humans, as well as non-human primates, domestic species, and default for everything that is not a mouse, rat, fish, worm, or fly, are capitalized and italicized (e.g., BMPR1A) and protein name abbreviations are capitalized, but not italicized (e.g., BMPR1A). Further, standard gene/protein nomenclature guidelines generally stipulate mouse, rat, and chicken gene name abbreviations/symbols italicized with the first letter only capitalized (e.g., Bmpr1a) and protein name abbreviations capitalized, but not italicized (e.g., Bmpr1a).

In contrast, the gene/protein nomenclature used herein when referencing a specific biomarker uses one with all capital letters (e.g., BMPR1A, PTHLH, ERG, SIX2, ALX4, BMP2, EFNB1, FGFR1, FGFR2, SMAD6, SPRY1, TWIST1, ZIC1, CREB3L1, ENG, CTGF, TEAD2, WWTR1, AMOTL1, SAV1, AMOTL2, TEAD1, HES1, PDGFRB, SIX1, SOX4, JAG1, MYLK, TBX2, and COL3A1) or one having lower letters (e.g., Bmpr1a, Pthlh, Erg, Six2, Alx4, Bmp2, Efnb1, Fgfr1, Fgfr2, Smad6, Spry1, Twist1, Zic1, Creb3l1, Eng, Ctgf, Tead2, Wwtr1, Amotl1, Sav1, Amotl2, Tead1, Hes1, Pdgfrb, Six1, Sox4, Jag1, Mylk, Tbx2, and Col3a1) for the biomarker abbreviation, is intended to be inclusive of genes (including mRNAs and cDNAs) and proteins across animal species. That is, when referring to a maker in this application, the name abbreviations/symbols in capital letters and in non-capital letters are used interchangeably unless their context indicates otherwise (e.g., in the working examples below, related figures, and related descriptions). Accordingly, the exemplary human or mouse biomarkers described herein are not intended to limit the present subject matter to human or mouse polypeptide biomarkers or mRNA biomarkers only. Rather, the present subject matter encompasses biomarkers across animal species that are associated with SSCs.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Unless otherwise stated, the term “about” means within an acceptable error range for the particular value.

### Examples

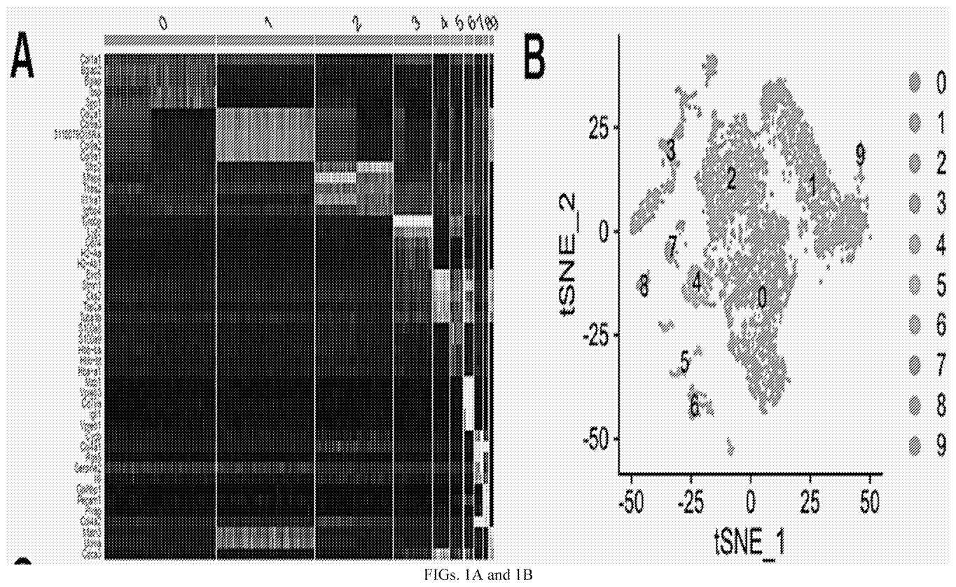

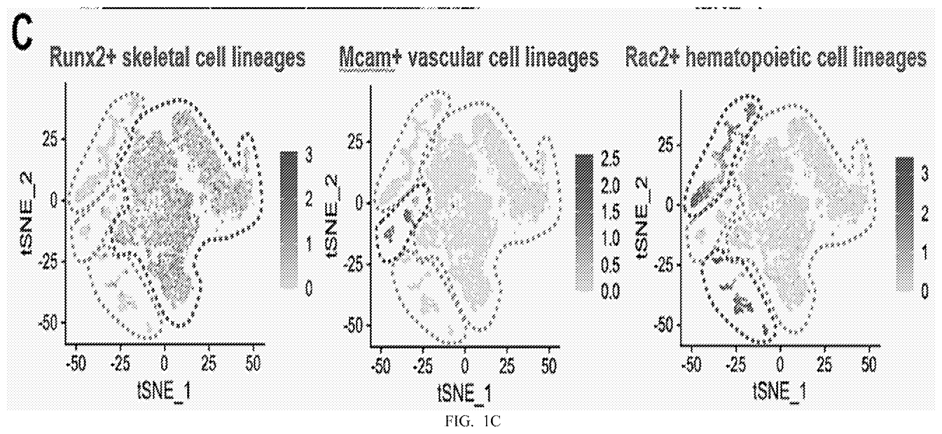

Using single-cell RNA-sequencing analysis, the heterogeneity of suture mesenchyme was first examined by defining the presence of multiple cell lineages. The visualization of the high dimensional t-distributed stochastic neighbor embedding (tSNE) algorithm grouped all the suture cells into ten distinct clusters (FIGS. 1A and 1B).

Next, the expressions of Runx2, Mcam, and Rac2 genes were used to subgroup them into three major lineages, skeletal cell lineages, vascular cell lineages, and hematopoietic cell lineages, respectively (FIG. 1C).

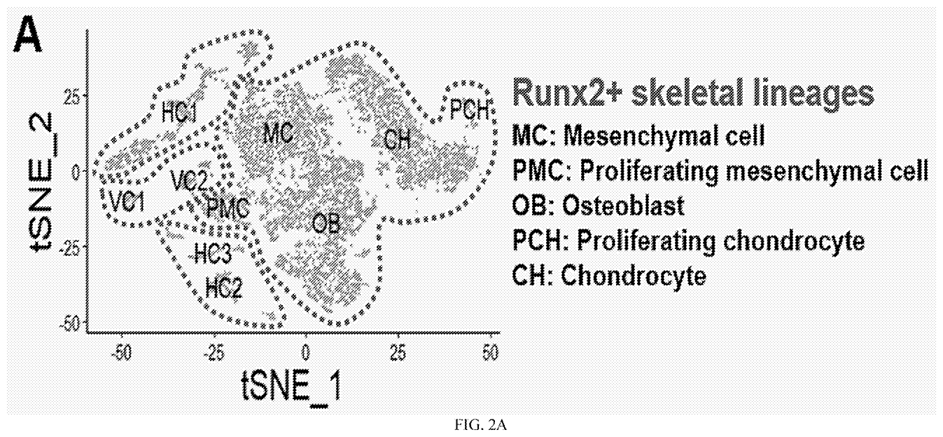

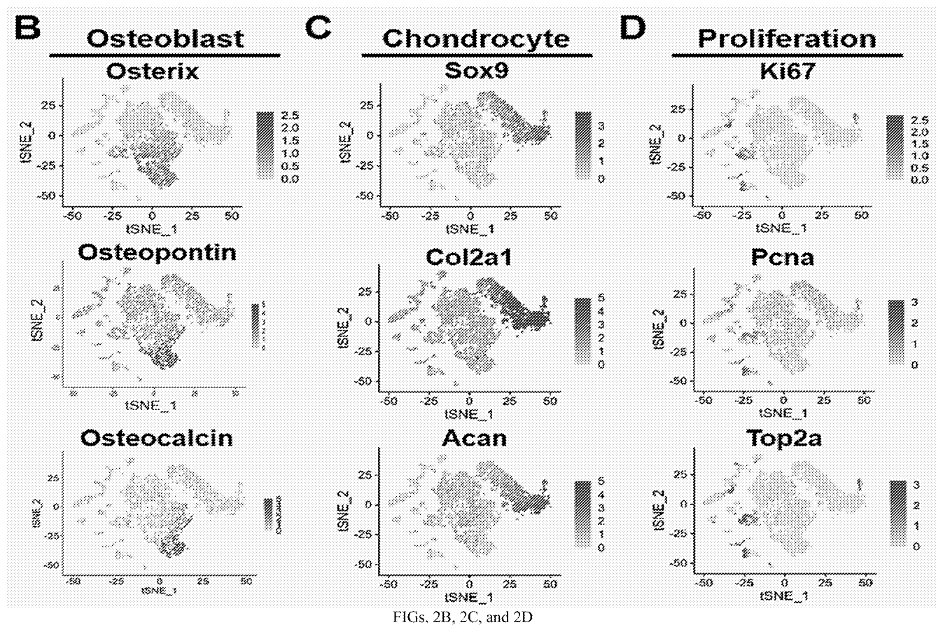

Then, the cell types present in the Runx2+ skeletal cell lineages were further examined and the identity of five clusters was revealed (FIG. 2A). Using markers expressed in various osteoblast and chondrocyte, their corresponding clusters were identified (FIGS. 2B and 2C). The clusters negative for osteoblast and chondrocyte markers represent the mesenchymal cell (MC) cluster.

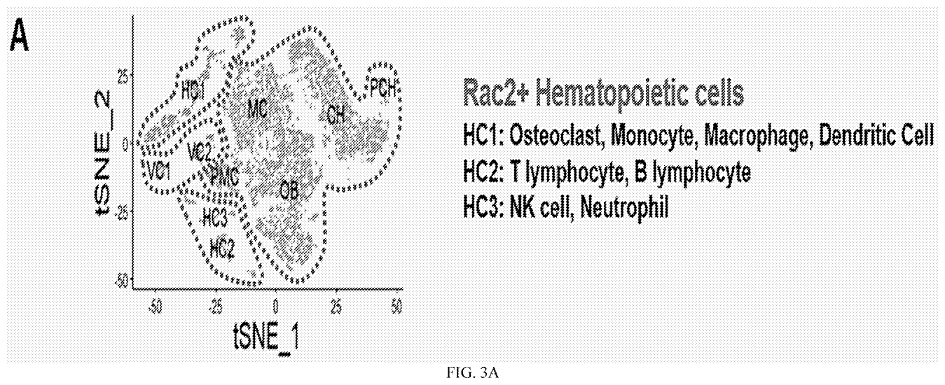

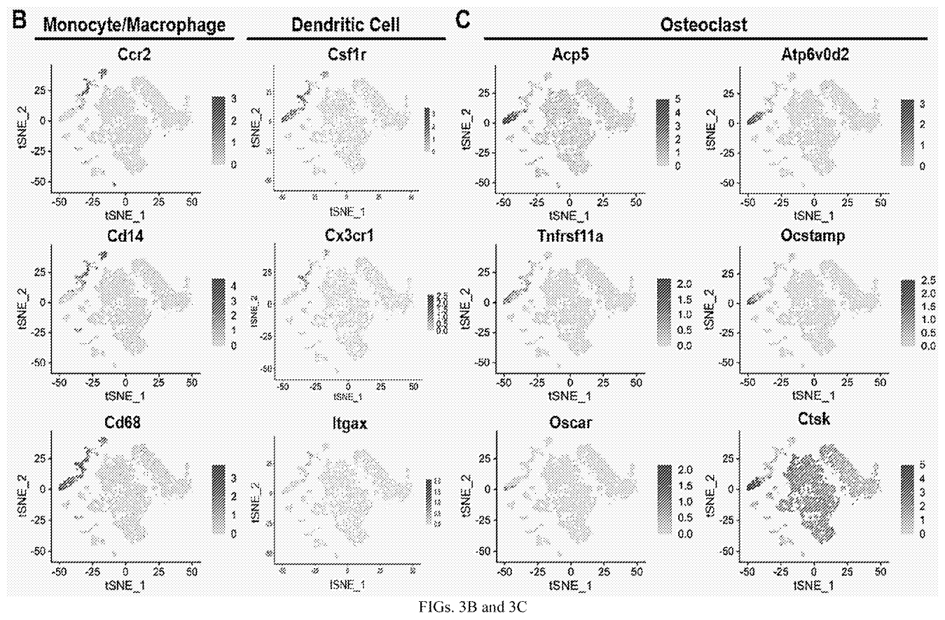

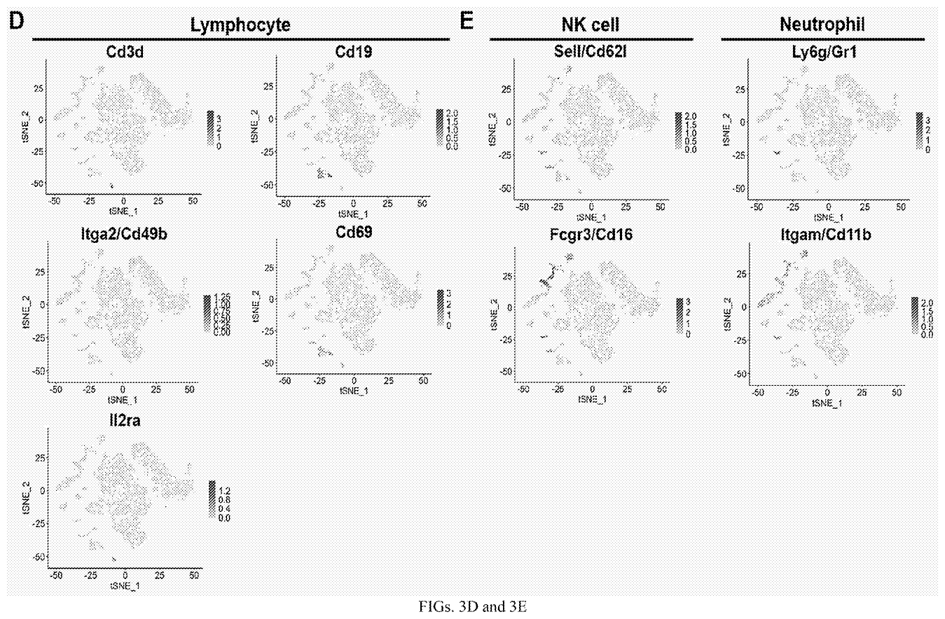

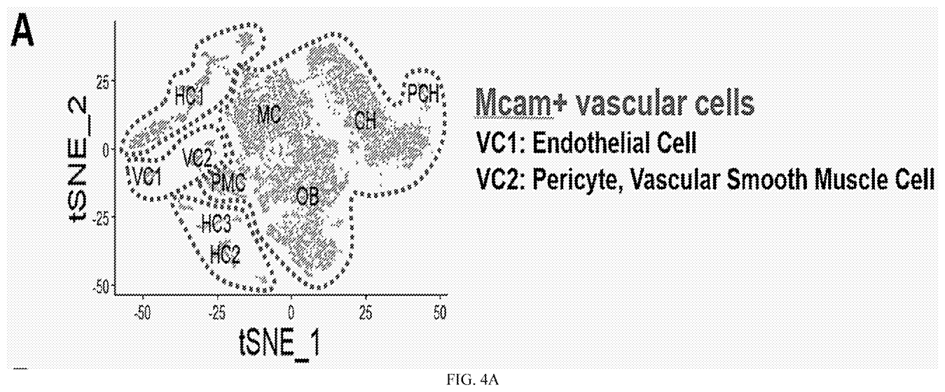

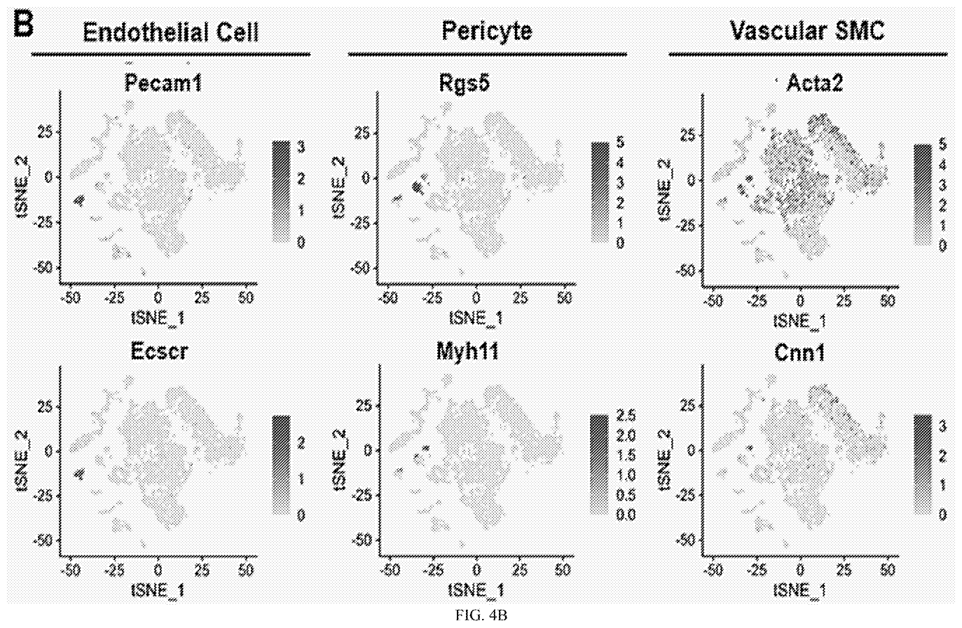

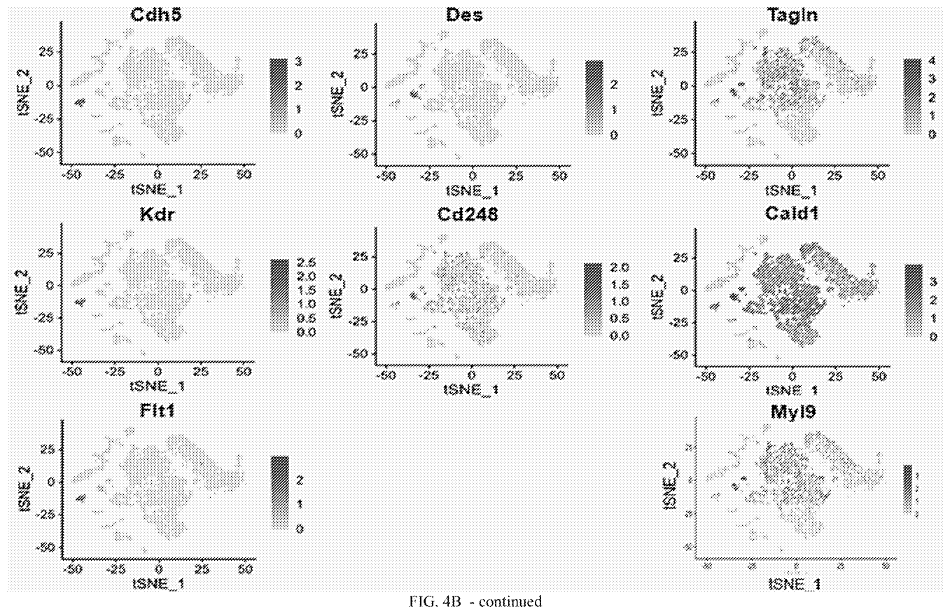

The cell proliferation markers were able to identify proliferating mesenchymal cells and proliferating chondrocyte clusters (FIG. 2D). Using similar approaches, three clusters of HCT-3 in the hematopoietic cell lineages were identified (FIG. 3A). The HC1 cluster consisted of closely related monocytes, macrophages, dendritic cells, and osteoclasts (FIGS. 3B and 3C). The HC2 cluster contained T and B lymphocytes while NK cells and Neutrophils were included in the HC3 cluster (FIGS. 3D and 3E). The vascular cell lineages contain three major cell types: endothelial cells, pericytes, and vascular smooth muscle cells. Genes expressed in these cells were used to characterize the two VCT-2 clusters of vascular cell lineages (FIG. 4A). The endothelial cell markers expressed highly in the VC1 cluster while the pericyte and vascular smooth muscle cells concentrated in the VC2 cluster (FIG. 4B).

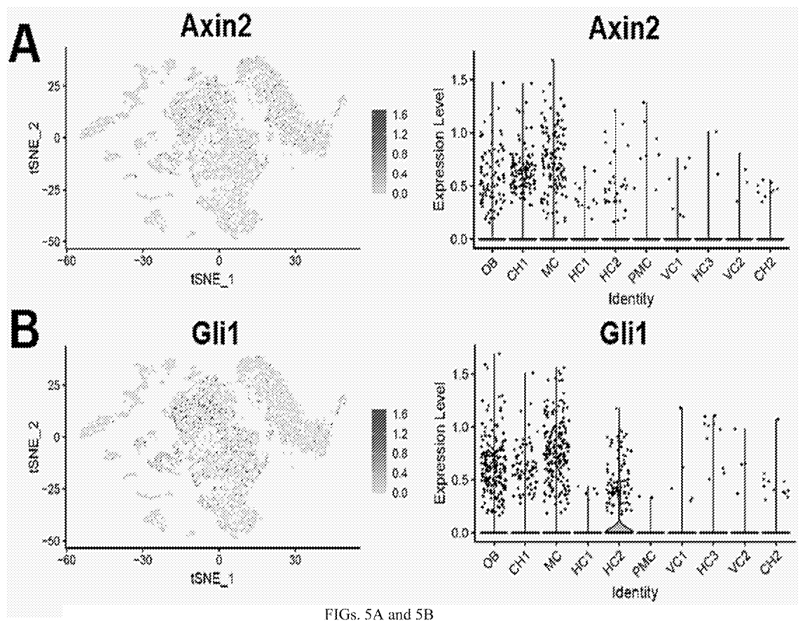

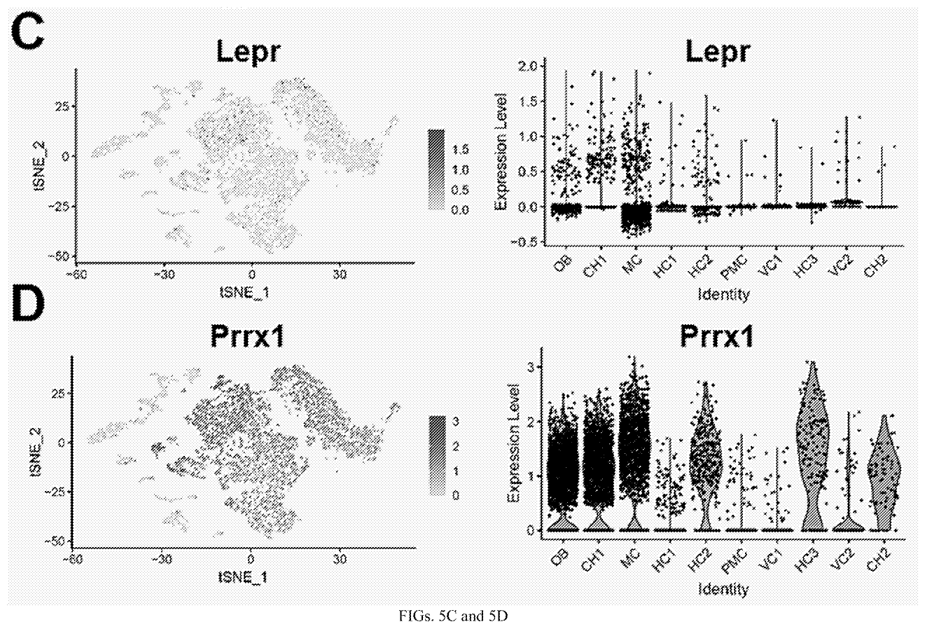

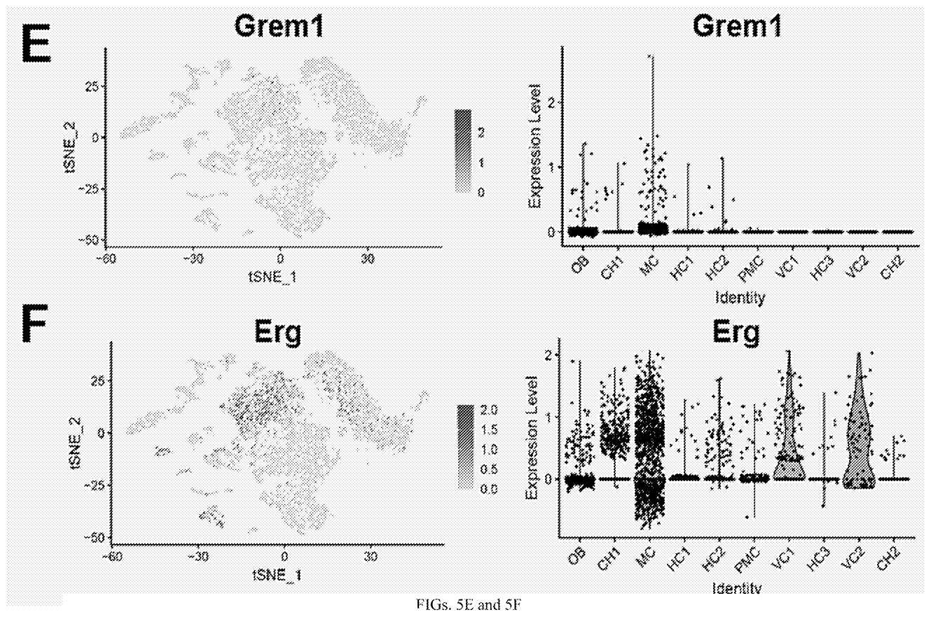

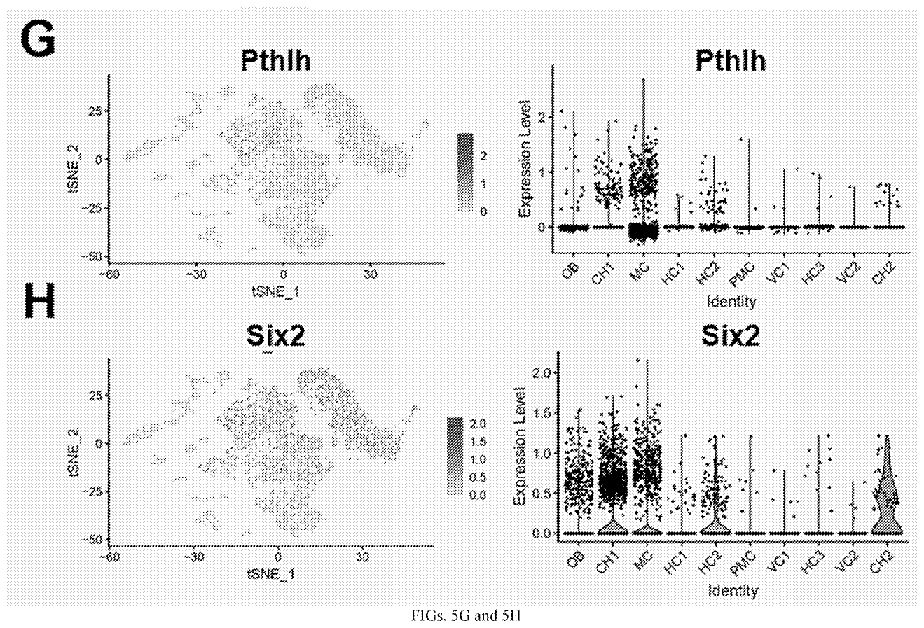

After the successful identification of all ten clusters, several known skeletal stem and progenitor cell markers were used to test if their positive cells are present in the expected MC cluster (FIG. 5). The expression of these marker genes, including Axin2, could all be found in the MC cluster. However, their expression was not restricted to the MC but could be identified in other clusters.

It was previously demonstrated that the Axin2-expressing cells are genuine skeletal stem cells capable of generating bone at the ectopic site and repairing large bone defects through direct engraftment and replacement of the damaged tissues. Therefore, assays were carried out to further characterize the Axin2+ cell population using single-cell RNA-sequencing (scRNA-seq) to refine the stem cell population thereby identifying potential stem cell markers and elucidating mechanisms underlying stem cell regulation.

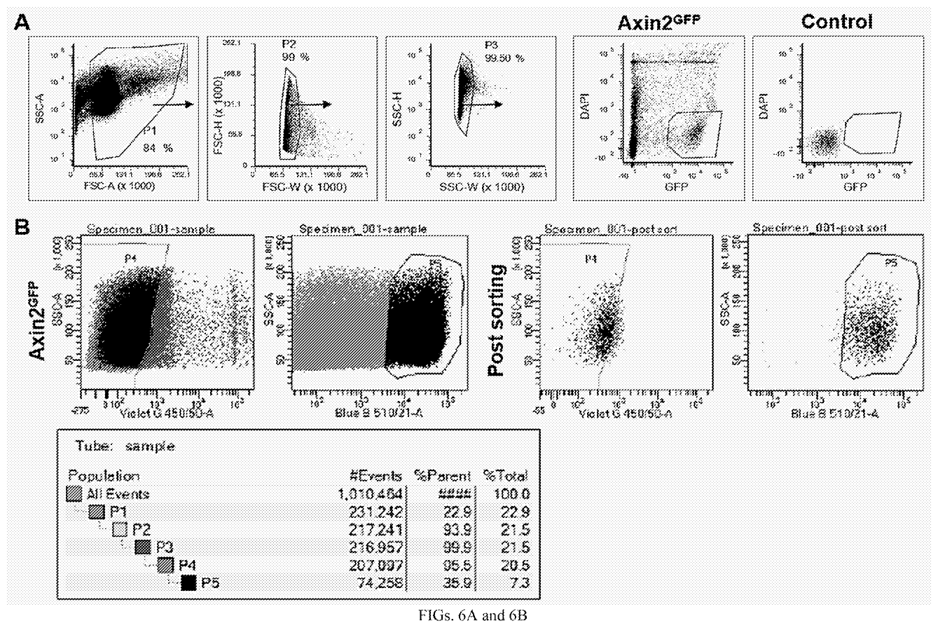

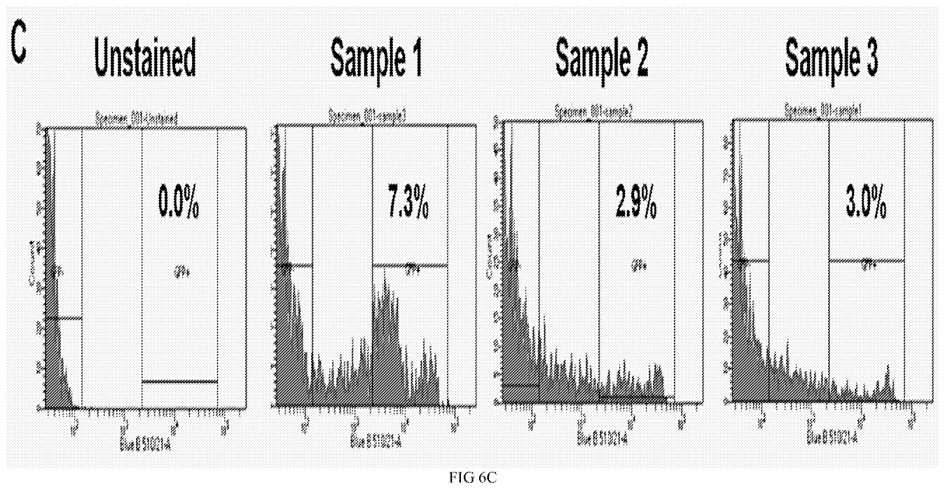

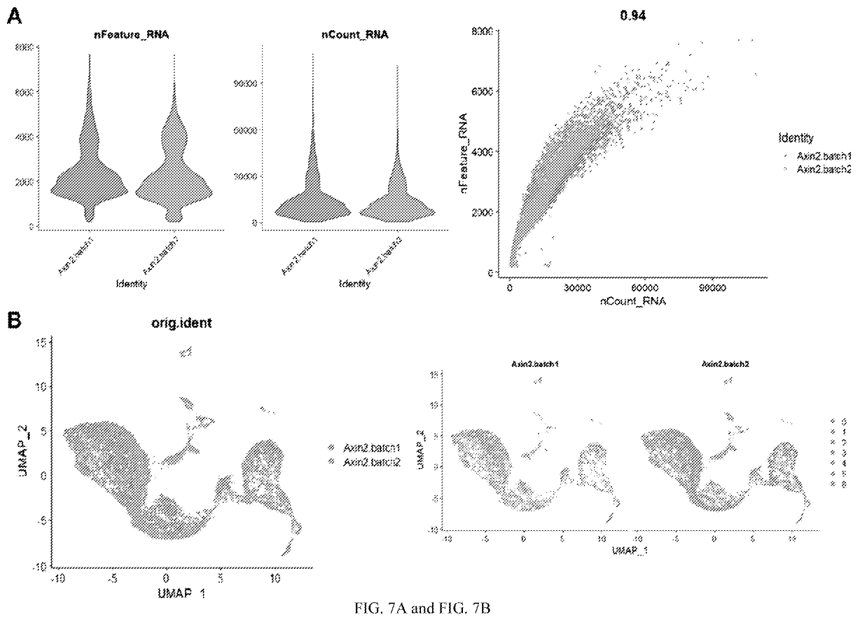

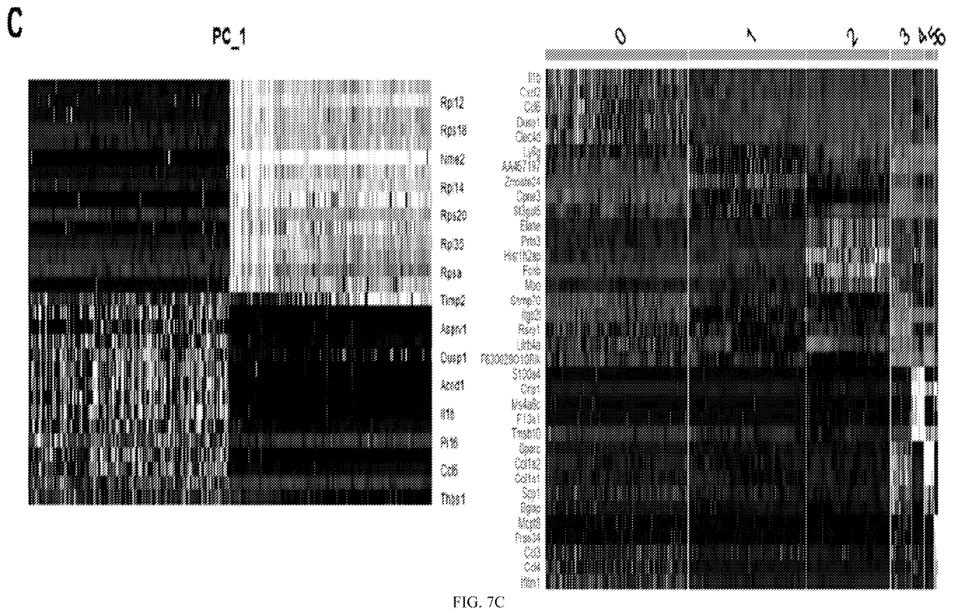

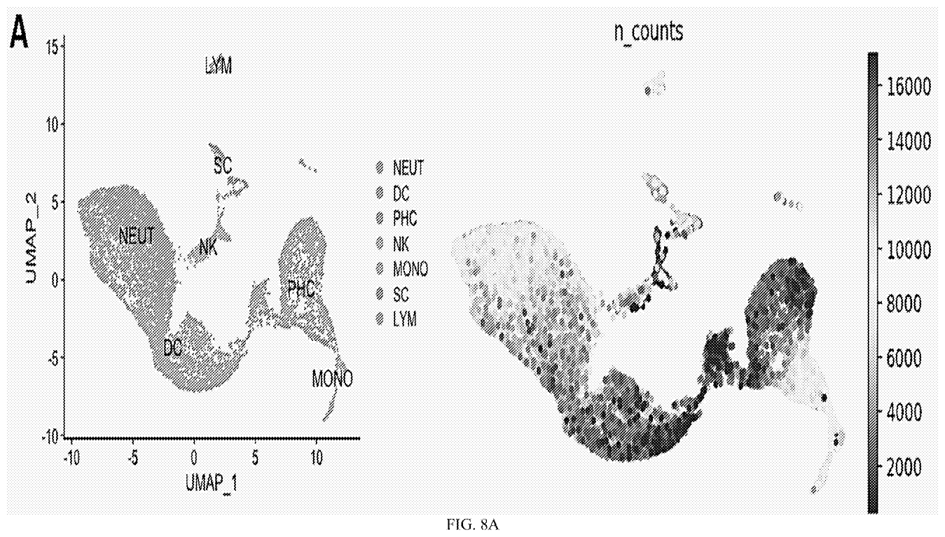

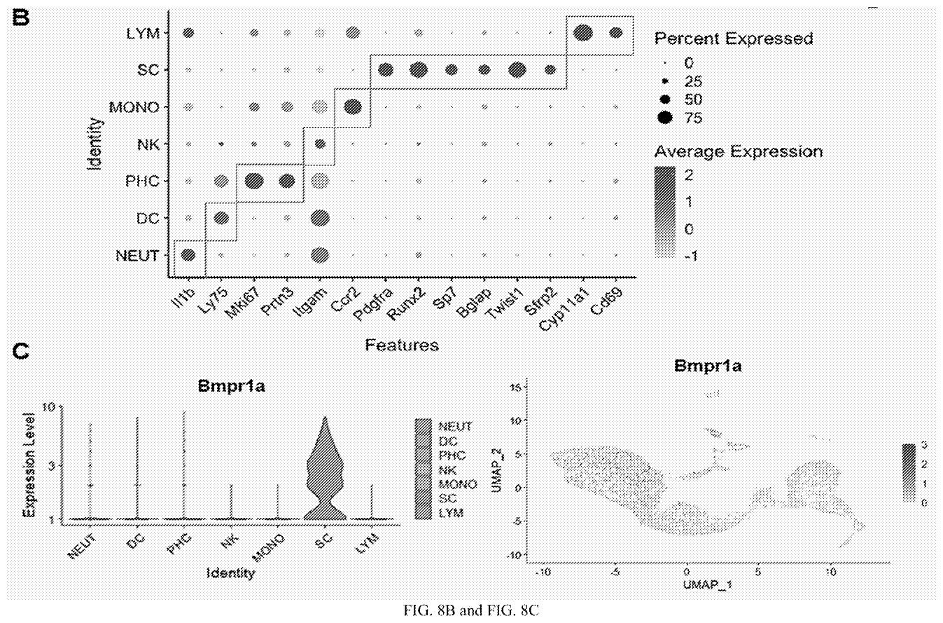

First, the Axin2+ cells were isolated from the calvarial suture using FACS based cell sorting (FIG. 6A). Consistent with previous findings, the Axin2+ cells consisted of 3-7% of the suture cell population (FIG. 6B). Next, single-cell RNA-seq analysis was performed to further examine the Axin2-expressing suture cells. The visualization of the high dimensional Uniform Manifold Approximation and Projection (UMAP) algorithm grouped the Axin2+ cell population into 7 clusters (FIG. 7). These clusters consist of neutrophils, dendritic cells, proliferating hematopoietic cells, natural killer cells, monocytes, skeletogenic cells, and lymphocytes based on the expression of gene markers (FIG. 8A-B).

As previously demonstrated that BMP type 1 receptor Bmpr1a is a stem cell marker, assays were carried out to examine which cluster contains Bmpr1a-expressing cells. Surprisingly, it was found that Bmpr1a+ cells were highly concentrated in the SC (skeletogenic cells) cluster containing only 244 cells, suggesting the enrichment of highly potent skeletal stem cells in this cluster (FIG. 8C).

The results thus permit the identification of genes expressed in the SC cluster as additional markers and potential regulators of skeletal stem cells in the suture. First, assays were carried out to examine genes in which their mutations have been linked to human patients with craniosynostosis, a devastating childhood disease caused by suture abnormality. Among 83 craniosynostosis genes examined, 27 of them, e.g., Fgfr1, Fgfr2, Twist1, Smo, Alx4, Bmp2, Bmper, Efnb1, Smad6, Spry1, Msx1, and Sox6, etc, are highly enriched in the SC cluster (FIG. 9). The rest of the 56 genes can be grouped into high enrichment in the SC but also present in other clusters (6 genes), low expression in the SC and other clusters (12 genes), and uniform expression in all clusters (38 genes).

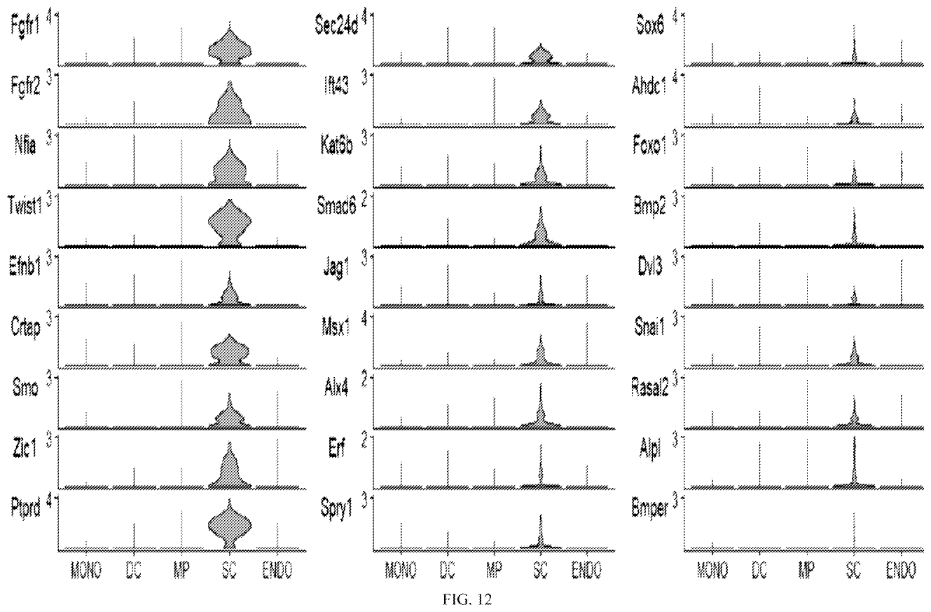

To further our examination of the SC cluster, re-clustering was performed to exclude cells from the hematopoietic lineage and refined the remaining cells into five subclusters (FIGS. 10A and 10B). The analysis of BMP signaling reveals its tight connection to the SC subcluster (FIG. 10C) solidifying previous findings of Bmpr1a essential for maintaining stem cell stemness. Therefore, skeletal stem cells are included in this cluster that is enriched with Bmpr1a expression. In addition, the expression of Bmp2, Acvr1, and Bmpr2 is highly restricted to the SC subcluster. A more detailed examination of genes associated with BMP signaling shows expression, e.g., Bmpr1a, Acvr1, Bambi, Bmp2, Bmp3, Bmp4, Bmp6, and Bmp7, in the SC subcluster (FIG. 11). Similar to that described in FIG. 9, 27 out of the 83 craniosynostosis genes also exhibited enriched distribution in the SC subcluster of the recluster plot (FIG. 12). 95 genes were identified specifically restricted to the SC subcluster (Table 2). Their gene products are membrane associated cell surface proteins, and thus can be used for skeletal stem cell purification.

TABLE 2
Exemplary Cell Surface Markers for SSCs

| | | Axin2.batch1_ | Axin2.batch2_ | | |
|---|---|---|---|---|---|
| cluster | gene | avg_log2FC | avg_log2FC | avg_log2FC | description |
| SC | Cd200 | 2.325809 | 1.560122 | 1.9429655 | CD200 antigen [Source: MGI |
| | | | | | Symbol; Acc: MGI: 1196990] |
| SC | Sdc2 | 1.628711 | 1.613614 | 1.6211625 | syndecan 2 [Source: MGI |
| | | | | | Symbol; Acc: MGI: 1349165] |
| SC | Mrc2 | 1.822855 | 1.203741 | 1.513298 | mannose receptor C type 2 |
| | | | | | [Source: MGI |
| | | | | | Symbol; Acc: MGI: 107818] |
| SC | Itgb1 | 1.454936 | 1.475095 | 1.4650155 | integrin beta 1 (fibronectin |
| | | | | | receptor beta) [Source: MGI |
| | | | | | Symbol; Acc: MGI: 96610] |
| SC | Pdgfra | 1.789857 | 0.9780301 | 1.38394355 | platelet derived growth factor |
| | | | | | receptor alpha polypeptide |
| | | | | | [Source: MGI |
| | | | | | Symbol; Acc: MGI: 97530] |
| SC | Fgfr2 | 1.151356 | 1.496494 | 1.323925 | fibroblast growth factor receptor |
| | | | | | 2 [Source: MGI |
| | | | | | Symbol; Acc: MGI: 95523] |
| SC | Pdgfrb | 1.160172 | 1.435946 | 1.298059 | platelet derived growth factor |
| | | | | | receptor beta polypeptide |
| | | | | | [Source: MGI |
| | | | | | Symbol; Acc: MGI: 97531] |
| SC | Cd63 | 1.461526 | 1.035491 | 1.2485085 | CD63 antigen [Source: MGI |
| | | | | | Symbol; Acc: MGI: 99529] |
| SC | Cdh2 | 1.18136 | 1.160741 | 1.1710505 | cadherin 2 [Source: MGI |
| | | | | | Symbol; Acc: MGI: 88355] |
| SC | Lamp1 | 1.152663 | 0.7238539 | 0.93825845 | lysosomal-associated membrane |
| | | | | | protein 1 [Source: MGI |
| | | | | | Symbol; Acc: MGI: 96745] |
| SC | Prnp | 0.8038233 | 0.9670312 | 0.88542725 | prion protein [Source: MGI |
| | | | | | Symbol; Acc: MGI: 97769] |
| SC | Bst2 | 0.6043616 | 0.7194218 | 0.6618917 | bone marrow stromal cell antigen |
| | | | | | 2 [Source: MGI |
| | | | | | Symbol; Acc: MGI: 1916800] |
| SC | Bmpr1a | 0.4779463 | 0.7121502 | 0.59504825 | bone morphogenetic protein |
| | | | | | receptor type 1A [Source: MGI |
| | | | | | Symbol; Acc: MGI: 1338938] |
| SC | Eng | 0.5891818 | 0.4738802 | 0.531531 | endoglin [Source: MGI |
| | | | | | Symbol; Acc: MGI: 95392] |
| SC | Itgav | 0.45296 | 0.5156581 | 0.48430905 | integrin alpha V [Source: MGI |
| | | | | | Symbol; Acc: MGI: 96608] |
| SC | Axl | 0.9320541 | 0.3693177 | 0.6506859 | AXL receptor tyrosine kinase |
| | | | | | [Source: MGI |
| | | | | | Symbol; Acc: MGI: 1347244] |
| SC | Sparc | 6.863901 | 6.553393 | 6.708647 | secreted acidic cysteine rich |
| | | | | | glycoprotein [Source: MGI |
| | | | | | Symbol; Acc: MGI: 98373] |
| SC | Bgr | 4.222109 | 4.015369 | 4.118739 | biglycan [Source: MGI |
| | | | | | Symbol; Acc: MGI: 88158] |
| SC | Lgals1 | 3.535151 | 3.499425 | 3.517288 | Lectin galactose binding soluble |
| | | | | | 1 [Source: MGI |
| | | | | | Symbol; Acc: MGI: 96777] |
| SC | Apoe | 2.610451 | 2.911694 | 2.7610725 | apolipoprotein E [Source: MGI |
| | | | | | Symbol; Acc: MGI: 88057] |
| SC | Itgb5 | 2.525375 | 2.243881 | 2.384628 | integrin beta 5 [Source: MGI |
| | | | | | Symbol; Acc: MGI: 96614] |
| SC | Fap | 2.791389 | 1.801161 | 2.296275 | fibroblast activation protein |
| | | | | | [Source: MGI |
| | | | | | Symbol; Acc: MGI: 109608] |
| SC | Tnn | 1.621926 | 2.911967 | 2.2669465 | tenascin N [Source: MGI |
| | | | | | Symbol; Acc: MGI: 2665790] |
| SC | Timp2 | 1.708933 | 2.026982 | 1.8679575 | tissue inhibitor of |
| | | | | | metalloproteinase 2 [Source: MGI |
| | | | | | Symbol; Acc: MGI: 98753] |
| SC | Mxra8 | 1.831679 | 1.887316 | 1.8594975 | matrix-remodelling associated 8 |
| | | | | | [Source: MGI |
| | | | | | Symbol; Acc: MGI: 1922011] |
| SC | Slit2 | 2.170464 | 1.346431 | 1.7584475 | slit guidance ligand 2 [Source: MGI |
| | | | | | Symbol; Acc: MGI: 1315205] |
| SC | Wif1 | 1.396075 | 1.906947 | 1.651511 | Wnt inhibitory factor 1 |
| | | | | | [Source: MGI |
| | | | | | Symbol; Acc: MGI: 1344332] |
| SC | App | 1.351537 | 1.691897 | 1.521717 | amyloid beta (A4) precursor |
| | | | | | protein [Source: MGI |
| | | | | | Symbol; Acc: MGI: 88059] |
| SC | Sdc4 | 1.485302 | 1.470472 | 1.477887 | syndecan 4 [Source: MGI |
| | | | | | Symbol; Acc: MGI: 1349164] |
| SC | Itgbl1 | 0.8646389 | 1.794989 | 1.32981395 | Integrin beta-like 1 [Source: MGI |
| | | | | | Symbol; Acc: MGI: 2443439] |
| SC | Fermt2 | 1.110471 | 1.400074 | 1.2552725 | fermitin family member 2 |
| | | | | | [Source: MGI |
| | | | | | Symbol; Acc: MGI: 2385001] |
| SC | Vasn | 1.319506 | 1.097773 | 1.2086395 | vasorin [Source: MGI |
| | | | | | Symbol; Acc: MGI: 2177651] |
| SC | Sfrp4 | 1.751158 | 0.6532206 | 1.2021893 | secreted frizzled-related protein 4 |
| | | | | | [Source: MGI |
| | | | | | Symbol; Acc: MGI: 892010] |
| SC | Pebp1 | 1.21277 | 1.132724 | 1.172747 | phosphatidylethanolamine |
| | | | | | binding protein 1 [Source: MGI |
| | | | | | Symbol; Acc: MGI: 1344408] |
| SC | Neo1 | 1.106821 | 1.195838 | 1.1513295 | neogenin [Source: MGI |
| | | | | | Symbol; Acc: MGI: 1097159] |
| SC | Calr | 1.079706 | 1.213593 | 1.1466495 | calreticulin [Source: MGI |
| | | | | | Symbol; Acc: MGI: 88252] |
| SC | Lrp4 | 1.352358 | 0.8907056 | 1.1215318 | low density lipoprotein receptor- |
| | | | | | related protein 4 [Source: MGI |
| | | | | | Symbol; Acc: MGI: 2442252] |
| SC | Pmp22 | 0.8088741 | 1.433843 | 1.12135855 | peripheral myelin protein 22 |
| | | | | | [Source: MGI |
| | | | | | Symbol; Acc: MGI: 97631] |
| SC | Antxr1 | 0.9347209 | 1.307089 | 1.12090495 | anthrax toxin receptor 1 |
| | | | | | [Source: MGI |
| | | | | | Symbol; Acc: MGI: 1916788] |
| SC | Lmo7 | 1.272348 | 0.9641834 | 1.1182657 | LIM domain only 7 [Source: MGI |
| | | | | | Symbol; Acc: MGI: 1353586] |
| SC | Ctsb | 1.089569 | 1.097394 | 1.0934815 | cathepsin B [Source: MGI |
| | | | | | Symbol; Acc: MGI: 88561] |
| SC | Robo2 | 1.405665 | 0.7626295 | 1.08414725 | roundabout guidance receptor 2 |
| | | | | | [Source: MGI |
| | | | | | Symbol; Acc: MGI: 1890110] |
| SC | Cryab | 1.049962 | 1.090837 | 1.0703995 | Crystallin alpha B [Source: MGI |
| | | | | | Symbol; Acc: MGI: 88516] |
| SC | Lpar1 | 1.13755 | 0.9980433 | 1.06779665 | lysophosphatidic acid receptor 1 |
| | | | | | [Source: MGI |
| | | | | | Symbol; Acc: MGI: 108429] |
| SC | Tgfbr3 | 1.286025 | 0.8309691 | 1.05849705 | transforming growth factor beta |
| | | | | | receptor III [Source: MGI |
| | | | | | Symbol; Acc: MGI: 104637] |
| SC | Hsp90ab1 | 1.266886 | 0.8464758 | 1.0566809 | heat shock protein 90 alpha |
| | | | | | (cytosolic) class B member 1 |
| | | | | | [Source: MGI |
| | | | | | Symbol; Acc: MGI: 96247] |
| SC | Enpp1 | 1.30798 | 0.7885909 | 1.04828545 | ectonucleotide |
| | | | | | pyrophosphatase/ |
| | | | | | phosphodiesterase 1 [Source: |
| | | | | | MGI Symbol; Acc: MGI: 97370] |
| SC | Il12a | 1.582085 | 0.5018337 | 1.04195935 | interleukin 12a [Source: MGI |
| | | | | | Symbol; Acc: MGI: 96539] |
| SC | Gpc3 | 1.099678 | 0.9792113 | 1.03944465 | glypican 3 [Source: MGI |
| | | | | | Symbol; Acc: MGI: 104903] |
| SC | Srpx2 | 1.112039 | 0.9640087 | 1.03802385 | sushi-repeat-containing protein X- |
| | | | | | linked 2 [Source: MGI |
| | | | | | Symbol; Acc: MGI: 1916042] |
| SC | Pdgfa | 1.061713 | 1.006015 | 1.033864 | platelet derived growth factor |
| | | | | | alpha [Source: MGI |
| | | | | | Symbol; Acc: MGI: 97527] |
| SC | Ghr | 0.9029858 | 1.097564 | 1.0002749 | growth hormone receptor |
| | | | | | [Source: MGI |
| | | | | | Symbol; Acc: MGI: 95708] |
| SC | Gpc1 | 1.094283 | 0.8837822 | 0.9890326 | glypican 1 [Source: MGI |
| | | | | | Symbol; Acc: MGI: 1194891] |
| SC | Pam | 0.675324 | 1.2711 | 0.973212 | peptidylglycine alpha-amidating |
| | | | | | monooxygenase [Source: MGI |
| | | | | | Symbol; Acc: MGI: 97475] |
| SC | Pdia3 | 0.889121 | 0.8930116 | 0.8910663 | protein disulfide isomerase |
| | | | | | associated 3 [Source: MGI |
| | | | | | Symbol; Acc: MGI: 95834] |
| SC | Lrpap1 | 0.8123232 | 0.9319639 | 0.87214355 | low density lipoprotein receptor- |
| | | | | | related protein associated protein |
| | | | | | 1 [Source: MGI |
| | | | | | Symbol; Acc: MGI: 96829] |
| SC | Sdc3 | 1.031845 | 0.7007873 | 0.86631615 | syndecan 3 [Source: MGI |
| | | | | | Symbol; Acc: MGI: 1349163] |
| SC | Emp2 | 0.9377905 | 0.7855405 | 0.8616655 | epithelial membrane protein 2 |
| | | | | | [Source: MGI |
| | | | | | Symbol; Acc: MGI: 1098726] |
| SC | Ctsz | 0.9153656 | 0.7886301 | 0.85199785 | cathepsin Z [Source: MGI |
| | | | | | Symbol; Acc: MGI: 1891190] |
| SC | Lrrc15 | 0.6559573 | 1.013289 | 0.83462315 | leucine rich repeat containing 15 |
| | | | | | [Source: MGI |
| | | | | | Symbol; Acc: MGI: 1921738] |
| SC | Tgfbr2 | 0.7996128 | 0.8451608 | 0.8223868 | transforming growth factor beta |
| | | | | | receptor II [Source: MGI |
| | | | | | Symbol; Acc: MGI: 98729] |
| SC | Pla2g5 | 1.182334 | 0.4591997 | 0.82076685 | phospholipase A2 group V |
| | | | | | [Source: MGI |
| | | | | | Symbol; Acc: MGI: 101899] |
| SC | Clic4 | 0.836104 | 0.7750661 | 0.80558505 | chloride intracellular channel 4 |
| | | | | | (mitochondrial) [Source: MGI |
| | | | | | Symbol; Acc: MGI: 1352754] |
| SC | Abca1 | 0.8493096 | 0.6809911 | 0.76515035 | ATP-binding cassette sub-family A |
| | | | | | (ABC1) member 1 [Source: MGI |
| | | | | | Symbol; Acc: MGI: 99607] |
| SC | Clstn1 | 0.7197439 | 0.7951574 | 0.75745065 | calsyntenin 1 [Source: MGI |
| | | | | | Symbol; Acc: MGI: 1929895] |
| SC | Bmpr2 | 0.6575656 | 0.8404253 | 0.74899545 | bone morphogenetic protein |
| | | | | | receptor type II (serine/threonine |
| | | | | | kinase) [Source: MGI |
| | | | | | Symbol; Acc: MGI: 1095407] |
| SC | Hspa5 | 0.8248324 | 0.6630392 | 0.7439358 | heat shock protein 5 [Source: MGI |
| | | | | | Symbol; Acc: MGI: 95835] |
| SC | Lpl | 0.7927618 | 0.6759635 | 0.73436265 | lipoprotein lipase [Source: MGI |
| | | | | | Symbol; Acc: MGI: 96820] |
| SC | Lrp6 | 0.5748864 | 0.8505162 | 0.7127013 | low density lipoprotein receptor- |
| | | | | | related protein 6 [Source: MGI |
| | | | | | Symbol; Acc: MGI: 1298218] |
| SC | Pdia4 | 0.8667785 | 0.5310539 | 0.6989162 | protein disulfide isomerase |
| | | | | | associated 4 [Source: MGI |
| | | | | | Symbol; Acc: MGI: 104864] |
| SC | Egfr | 0.7831459 | 0.5660558 | 0.67460085 | epidermal growth factor receptor |
| | | | | | [Source: MGI |
| | | | | | Symbol; Acc: MGI: 95294] |
| SC | Fzd1 | 0.4188033 | 0.8999994 | 0.65940135 | frizzled class receptor 1 |
| | | | | | [Source: MGI |
| | | | | | Symbol; Acc: MGI: 1196625] |
| SC | Fzd5 | 0.6214266 | 0.6858438 | 0.6536352 | frizzled class receptor 5 |
| | | | | | [Source: MGI |
| | | | | | Symbol; Acc: MGI: 108571] |
| SC | Nptn | 0.4756922 | 0.8165528 | 0.6461225 | neuroplastin [Source: MGI |
| | | | | | Symbol; Acc: MGI: 108077] |
| SC | Tgfb3 | 0.5449818 | 0.710766 | 0.6278739 | transforming growth factor beta 3 |
| | | | | | [Source: MGI |
| | | | | | Symbol; Acc: MGI: 98727] |
| SC | Cd59a | 0.711373 | 0.5296984 | 0.6205357 | CD59a antigen [Source: MGI |
| | | | | | Symbol; Acc: MGI: 109177] |
| SC | Pcsk6 | 0.7306713 | 0.4752861 | 0.6029787 | proprotein convertase |
| | | | | | subtilisin/kexin type 6 |
| | | | | | [Source: MGI |
| | | | | | Symbol; Acc: MGI: 102897] |
| SC | Sema6d | 0.6954291 | 0.4791795 | 0.5873043 | sema domain transmembrane |
| | | | | | domain (TM) and cytoplasmic |
| | | | | | domain (semaphorin) 6D |
| | | | | | [Source: MGI |
| | | | | | Symbol; Acc: MGI: 2387661] |
| SC | Pkd1 | 0.5933907 | 0.555174 | 0.57428235 | polycystin 1 transient receptor |
| | | | | | potential channel interacting |
| | | | | | [Source: MGI |
| | | | | | Symbol; Acc: MGI: 97603] |
| SC | Bace1 | 0.6104902 | 0.5344182 | 0.5724542 | beta-site APP cleaving enzyme 1 |
| | | | | | [Source: MGI |
| | | | | | Symbol; Acc: MGI: 1346542] |
| SC | Sulf2 | 0.4960183 | 0.6382394 | 0.56712885 | sulfatase 2 [Source: MGI |
| | | | | | Symbol; Acc: MGI: 1919293] |
| SC | Mif | 0.629365 | 0.4884263 | 0.55889565 | macrophage migration inhibitory |
| | | | | | factor (glycosylation-inhibiting |
| | | | | | factor) [Source: MGI |
| | | | | | Symbol; Acc: MGI: 96982] |
| SC | Cdon | 0.531962 | 0.5205671 | 0.52626455 | cell adhesion molecule- |
| | | | | | related/down-regulated by |
| | | | | | oncogenes [Source: MGI |
| | | | | | Symbol; Acc: MGI: 1926387] |
| SC | Ppfia2 | 0.6367636 | 0.4064795 | 0.52162155 | protein tyrosine phosphatase |
| | | | | | eceptor type f polypeptide |
| | | | | | (PTPRF) interacting protein (liprin) |
| | | | | | alpha 2 [Source: MGI |
| | | | | | Symbol; Acc: MGI: 2443834] |
| SC | Tjp1 | 0.5568973 | 0.4476196 | 0.50225845 | tight junction protein 1 |
| | | | | | [Source: MGI |
| | | | | | Symbol; Acc: MGI: 98759] |
| SC | Lipc | 0.5155569 | 0.4487237 | 0.4821403 | Lipase hepatic [Source: MGI |
| | | | | | Symbol; Acc: MGI: 96216] |
| SC | Ncl | 0.4914069 | 0.4603604 | 0.47588365 | nucleolin [Source: MGI |
| | | | | | Symbol; Acc: MGI: 97286] |
| SC | Lrig3 | 0.3208133 | 0.6255536 | 0.47318345 | leucine-rich repeats and |
| | | | | | immunoglobulin-like domains 3 |
| | | | | | [Source: MGI |
| | | | | | Symbol; Acc: MGI: 2443955] |
| SC | Gpc4 | 0.5037107 | 0.4384532 | 0.47108195 | glypican 4 [Source: MGI |
| | | | | | Symbol; Acc: MGI: 104902] |
| SC | Adgra2 | 0.3543703 | 0.536139 | 0.44525465 | adhesion G protein-coupled |
| | | | | | receptor A2 [Source: MGI |
| | | | | | Symbol; Acc: MGI: 1925810] |
| SC | Boc | 0.4212035 | 0.4397791 | 0.4304913 | biregional cell adhesion molecule- |
| | | | | | related/down-regulated by |
| | | | | | oncogenes (Cdon) binding protein |
| | | | | | [Source: MGI |
| | | | | | Symbol; Acc: MGI: 2151153] |
| SC | Ptprk | 0.4264325 | 0.4187075 | 0.42257 | protein tyrosine phosphatase |
| | | | | | receptor type K [Source: MGI |
| | | | | | Symbol; Acc: MGI: 103310] |
| SC | Aamp | 0.4269861 | 0.4137104 | 0.42034825 | angio-associated migratory |
| | | | | | protein [Source: MGI Symbol; |
| | | | | | Acc: MGI: 107809] |
| SC | Anxa4 | 0.3572791 | 0.4240171 | 0.3906481 | annexin A4 [Source: MGI |
| | | | | | Symbol; Acc: MGI: 88030] |
| SC | C1qbp | 0.3688876 | 0.3541226 | 0.3615051 | complement component 1 q |
| | | | | | subcomponent binding protein |
| | | | | | [Source: MGI Symbol; |
| | | | | | Acc: MGI: 1194505] |

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A method for identifying or isolating or enriching a skeletal stem cell, comprising
obtaining a start cell population; and
identifying from the start cell population a first marker expressed by a cell, wherein the first marker is selected from the group consisting of Bmpr1a, Bmpr2, Fdz1, Fgfr2, Lrig3, Itgbl1, Apoe, Gpc3, Lpl, Sulf2, Cdon, Tgfbr2, Tgfbr3, Lrrc15, Mif, Axl, Itgav, Fgfr1, Jag1, Acvr1, Acvr2a, Acvr2b, Erg, Six2, Pthlh, Twist1, Alpl, Msx1, Efnb1, Zic1, Spry1, Abcc9, Erf, Bmp2, Bmp3, Bmp4, Bmp5, Bmp6, Bmp7, Bmp8a, Bambi, Bmper, and Col3a1.

2. The method of claim 1, further comprising isolating the cell expressing the first marker.

3. The method of claim 1, further comprising collecting a plurality of cells expressing the first marker to obtain an enriched skeletal stem cell population.

4. The method of claim 1, wherein the cell or cells are identified, isolated or enriched using a first protein, a first polypeptide, a first nucleic acid, or a first composition that specifically binds to the first marker.

5. The method of claim 1, further comprising identifying from the start cell population a second marker expressed by the cell expressing the first marker, wherein the second marker is selected from the group.

6. The method of claim 5, wherein the cell is identified, isolated or enriched using a second protein, a second polypeptide, a second nucleic acid, or a second composition that specifically binds to the second marker.

7. The method of claim 6, further comprising collecting a plurality of cells expressing the first and second markers to obtain an enriched skeletal stem cell population.

8. The method of claim 6, further comprising identifying from the start cell population one or more additional markers expressed by the cell co-expressing both the first and the second markers, wherein each of the one or more additional markers is individually identified using the same method as for the first and the second markers.

9. The method of claim 8 resulting in a cell co-expressing the first, the second and one or more additional markers.

10. The method of claim 9, further comprising collecting a plurality of cells expressing the first, the second, and one or more markers to obtain an enriched skeletal stem cell population.

11. The method of claim 1, wherein the start cell population is from a tissue of a subject.

12. The method of claim 11, wherein the subject is a mammal.

13. The method of claim 12, wherein the mammal is a human.

14. The method of claim 1, wherein the start cell population comprises one or more selected from the group consisting of bone marrow, cord blood cells, embryonic stem cells or progenies thereof, mesenchymal stem cells or progenies thereof, and induced pluripotent stem cells (iPSCs) or progenies thereof.

15. The method of claim 14, wherein the mesenchymal stem cells are suture mesenchymal stem cells.

16. A composition comprising (i) a carrier and (ii) one or more cells identified, isolated, or enriched according to the method of claim 1, or cells derived therefrom.

17. The composition of claim 16, wherein the composition is a pharmaceutical composition and the carrier is pharmaceutically acceptable carrier, or the composition is an in vitro cell culture composition and the carrier comprises a culture medium.

18. (canceled)

19. A bone regeneration product or formulation comprising (i) the composition of claim 16 and (ii) a scaffold.

20. A method for generating or regenerating cartilage or bone in a subject, comprising administering to a subject in need thereof an effective amount of the composition of claim 16 or a bone regeneration product or formulation comprising (i) the composition and (ii) a scaffold at a site where regeneration of bone or cartilage is desired.

21. A method of generating skeletal, stromal, or cartilaginous tissue, comprising
(i) providing one or more cells obtained according to claim 1, and
(ii) inducing differentiation of the one or more cells or differentiation of cells derived therefrom.

22. (canceled)
